(19) United States
(12) Patent Application Publication
(10) Pub. No.: US 2025/0017912 A1
(43) Pub. Date: Jan. 16, 2025

(54) METHODS FOR DOWN-REGULATION AND UP-REGULATION OF PATHWAYS

(71) Applicant: MedRegen, LLC, Baltimore, MD (US)

(72) Inventors: Zhaoli Sun, Perry Hall, MD (US); Ali Reza Ahmadi, Ridderkerk (NL)

(73) Assignee: MedRegen, LLC, Baltimore, MD (US)

(21) Appl. No.: 18/768,644

(22) Filed: Jul. 10, 2024

Related U.S. Application Data
(60) Provisional application No. 63/512,767, filed on Jul. 10, 2023.

(51) Int. Cl. A61K 31/436; A61K 31/395; A61P 1/16; A61P 25/28; A61P 37/06

(52) U.S. Cl. CPC A61K 31/436; A61K 31/395; A61P 1/16; A61P 25/28; A61P 37/06

(57) ABSTRACT

The present disclosure provides methods for treating various diseases or disorders, down-regulating and up-regulating various pathways and mobilizing stem and/or immune cells in a patient, comprising administering a pharmaceutical composition to a subject. In certain embodiments, the pharmaceutical composition comprises (a) a stem cell mobilizer and (b) an immunosuppressive agent or non-immunosuppressive FK binding protein ligand in a low or sub-immunosuppressive dose. The methods of the disclosure can comprise administering the pharmaceutical composition by routes and dosing regimens, as disclosed herein.

### FIELD OF THE DISCLOSURE

The present disclosure relates to methods of down-regulating and up-regulating pathways in a subject to treat various illnesses, diseases, disorders or other ailments.

### BACKGROUND OF THE DISCLOSURE

What is needed are useful and/or effective methods for various treatments for a subject, including to mobilize various stem and immune cells, to down-regulate molecular pathways for treating one or more of inflammation, allograft rejection and autoimmune disease in a subject, to down-regulate molecular pathways for one or more of: treating ischemia, reperfusion injury, neurodegenerative disease or disorder, inflammatory pain or disorder and steatohepatitis, anti-thrombosis, anti-aging, anti-fibrosis and anti-cancer, to up-regulate interferon alpha response pathway for stimulating an antiviral and anti-cancer response in a subject, and to up-regulate heme metabolism pathway for promoting heme synthesis, among others.

### SUMMARY OF THE DISCLOSURE

In one embodiment, the present disclosure provides methods to mobilize various stem and immune cells in a subject. In one embodiment, the present disclosure provides methods to down-regulate molecular pathways for treating one or more of inflammation, allograft rejection and autoimmune disease in a subject. In one embodiment, the present disclosure provides method to down-regulate molecular pathways for one or more of: treating ischemia, reperfusion injury, neurodegenerative disease or disorder, inflammatory pain or disorder and steatohepatitis, anti-thrombosis, anti-aging, anti-fibrosis and anti-cancer, in a subject. In one embodiment, the present disclosure provides methods to up-regulate interferon alpha response pathway for stimulating an antiviral and anti-cancer response in a subject. In one embodiment, the present disclosure provides methods to up-regulate heme metabolism pathway for promoting heme synthesis in a subject.

In one embodiment, the methods of the present disclosure comprise administering to a subject a pharmaceutical composition comprising a stem cell mobilizer, and an immunosuppressive agent or non-immunosuppressive FK binding protein ligand. In one embodiment, the immunosuppressive agent or non-immunosuppressive FK binding protein ligand is administered at a sub-immunosuppressive dose. In an embodiment, the stem cell mobilizer comprises AMD 3100.

In one embodiment, the dose range of the stem cell mobilizer, or AMD3100, is about 0.12 mg/kg to about 0.48 mg/kg. In one embodiment, the dose of the stem cell mobilizer, or AMD3100, is about 0.14 mg/kg, about 0.16 mg/kg, about 0.18 mg/kg, about 0.20 mg/kg, about 0.22 mg/kg, about 0.24 mg/kg, about 0.26 mg/kg, about 0.28 mg/kg, about 0.30 mg/kg, about 0.32 mg/kg, about 0.34 mg/kg, about 0.36 mg/kg, about 0.38 mg/kg, about 0.40 mg/kg, about 0.42 mg/kg, about 0.44 mg/kg, about 0.46 mg/kg, or about 0.48 mg/kg. In one embodiment, the stem cell mobilizer, or AMD3100, plasma concentration range at about 1, about 3, and/or about 8 hours is about 200 ng/mL to about 1,000 ng/mL. In one embodiment, the stem cell mobilizer, or AMD3100, plasma concentration at about 1, about 3, and/or about 8 hours is about 200 ng/ml, about 250 ng/ml, about 300 ng/mL, about 350 ng/ml, about 400 ng/ml, about 450 ng/ml, about 500 ng/mL, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml or about 1,000 ng/mL.

In one embodiment, the immunosuppressive agent or non-immunosuppressive FK binding protein ligand comprises Tacrolimus. In one embodiment, the dose range of the immunosuppressive agent or non-immunosuppressive FK binding protein ligand, or Tacrolimus, is about 0.0025 mg/kg to about 0.01 mg/kg. In one embodiment, the dose of the immunosuppressive agent or non-immunosuppressive FK binding protein ligand, or Tacrolimus, is about 0.0030 mg/kg, about 0.0035 mg/kg, about 0.0040 mg/kg, about 0.0045 mg/kg, about 0.0050 mg/kg, about 0.0055 mg/kg, about 0.0060 mg/kg, about 0.0065 mg/kg, about 0.0070 mg/kg, about 0.0075 mg/kg, about 0.0080 mg/kg, about 0.0085 mg/kg, about 0.0090 mg/kg, about 0.0095 mg/kg, or about 0.01 mg/kg.

In one embodiment, the immunosuppressive agent or non-immunosuppressive FK binding protein ligand, or Tacrolimus, plasma concentration range at about 1, about 3, about 8, about 12 and/or about 24 hours is about 1 ng/ml to about 7 ng/ml. In one embodiment, the immunosuppressive agent or non-immunosuppressive FK binding protein ligand, or Tacrolimus, plasma concentration range at about 1, about 3, about 8, about 12 and/or about 24 hours is about 1 ng/ml, about 2 ng/ml, about 3 ng/ml, about 4 ng/ml, about 5 ng/mL, about 6 ng/ml or about 7 ng/mL.

### BRIEF DESCRIPTION OF THE FIGURES

FIG. 1A shows time profiles for plerixafor following SC administration of a single dose of 0.005, 0.01, and 0.02 mL/kg Composition A on Day 1, as describe in Example 1, according to an exemplary embodiment of the present disclosure.

FIG. 1B shows time profiles for plerixafor following QAD SC administration of 0.005, 0.01, and 0.02 mL/kg Composition A on day 5, as described in Example 1, according to an exemplary embodiment of the present disclosure.

FIG. 1C shows time profiles for tacrolimus following SC administration of a single dose of 0.005, 0.01, and 0.02 mL/kg Composition A on Day 1, as described in Example 1, according to an exemplary embodiment of the present disclosure.

FIG. 1D shows time profiles for tacrolimus following QAD SC administration for 5 days of 0.005, 0.01, and 0.02 mL/kg Composition A on Day 5. n=4 per group, as described in Example 1, according to an exemplary embodiment of the present disclosure.

FIGS. 2A-2L show white blood cell mobilization with Composition A in healthy subjects, as described in Example 1, according to an exemplary embodiment of the present disclosure.

FIG. 2A shows leukocytes on day 1 following a single dose SC injection of saline or Composition A, as described in Example 1, according to an exemplary embodiment of the present disclosure.

FIG. 2B shows neutrophils on day 1 following a single dose SC injection of saline or Composition A, as described in Example 1, according to an exemplary embodiment of the present disclosure.

FIG. 2C shows lymphocytes on day 1 following a single dose SC injection of saline or Composition A, as described in Example 1, according to an exemplary embodiment of the present disclosure.

FIG. 2D shows monocytes on day 1 following a single dose SC injection of saline or Composition A, as described in Example 1, according to an exemplary embodiment of the present disclosure.

FIG. 2E shows eosinophils on day 1 following a single dose SC injection of saline or Composition A, as described in Example 1, according to an exemplary embodiment of the present disclosure.

FIG. 2F shows basophils on day 1 following a single dose SC injection of saline or Composition A, as described in Example 1, according to an exemplary embodiment of the present disclosure.

FIG. 2G shows leukocytes on day 5 following the third dose SC injection of saline or Composition A, as described in Example 1, according to an exemplary embodiment of the present disclosure.

FIG. 2H shows neutrophils on day 5 following the third dose SC injection of saline or Composition A, as described in Example 1, according to an exemplary embodiment of the present disclosure.

FIG. 2I shows lymphocytes on day 5 following the third dose SC injection of saline or Composition A, as described in Example 1, according to an exemplary embodiment of the present disclosure.

FIG. 2J shows monocytes on day 5 following the third dose SC injection of saline or Composition A, as described in Example 1, according to an exemplary embodiment of the present disclosure.

FIG. 2K shows eosinophils on day 5 following the third dose SC injection of saline or Composition A, as described in Example 1, according to an exemplary embodiment of the present disclosure.

FIG. 2L shows basophils on day 5 following the third dose SC injection of saline or Composition A, as described in Example 1, according to an exemplary embodiment of the present disclosure.

FIGS. 3A-3L show lymphocyte mobilization with Composition A in healthy subjects, as described in Example 1, according to an exemplary embodiment of the present disclosure.

FIG. 3A shows CD3+ populations in peripheral blood on day 1 following a single dose SC injection of saline or Composition A, as described in Example 1, according to an exemplary embodiment of the present disclosure.

FIG. 3B shows CD3+CD4+ populations in peripheral blood on day 1 following a single dose SC injection of saline or Composition A, as described in Example 1, according to an exemplary embodiment of the present disclosure.

FIG. 3C shows CD3+CD8+ populations in peripheral blood on day 1 following a single dose SC injection of saline or Composition A, as described in Example 1, according to an exemplary embodiment of the present disclosure.

FIG. 3D shows CD19+ populations in peripheral blood on day 1 following a single dose SC injection of saline or Composition A, as described in Example 1, according to an exemplary embodiment of the present disclosure.

FIG. 3E shows CD3+CD4+FOXP3+ populations in peripheral blood on day 1 following a single dose SC injection of saline or Composition A, as described in Example 1, according to an exemplary embodiment of the present disclosure.

FIG. 3F shows CD3+CD8+FOXP3+ populations in peripheral blood on day 1 following a single dose SC injection of saline or Composition A, as described in Example 1, according to an exemplary embodiment of the present disclosure.

FIG. 3G shows CD3+ populations in peripheral blood on day 5 following the third dose. Each value represents the mean±SD, as described in Example 1, according to an exemplary embodiment of the present disclosure.

FIG. 3H shows CD3+CD4+ populations in peripheral blood on day 5 following the third dose. Each value represents the mean±SD, as described in Example 1, according to an exemplary embodiment of the present disclosure.

FIG. 3I shows CD3+CD8+ populations in peripheral blood on day 5 following the third dose. Each value represents the mean±SD, as described in Example 1, according to an exemplary embodiment of the present disclosure.

FIG. 3J shows CD 19+ populations in peripheral blood on day 5 following the third dose. Each value represents the mean±SD, as described in Example 1, according to an exemplary embodiment of the present disclosure.

FIG. 3K shows CD3+CD4+FOXP3+ populations in peripheral blood on day 5 following the third dose. Each value represents the mean±SD, as described in Example 1, according to an exemplary embodiment of the present disclosure.

FIG. 3L shows CD3+CD8+FOXP3+ populations in peripheral blood on day 5 following the third dose. Each value represents the mean±SD, as described in Example 1, according to an exemplary embodiment of the present disclosure.

FIG. 3M shows the percent change from baseline (PCFB) over time (hours) of CD3+CD4+CCR7+CD45RA⁻ population with 0.01 mL/kg of Composition A, according to an exemplary embodiment of the present disclosure.

FIG. 3N shows the percent change from baseline (PCFB) over time (hours) of CD3+CD4+CCR7+CD45RA⁻ population with 0.01 mL/kg of Composition A, according to an exemplary embodiment of the present disclosure.

FIG. 3O shows the percent change from baseline (PCFB) over time (hours) of CD3+CD4+CCR7+CD45RA⁻ population with 0.01 mL/kg of Composition A, according to an exemplary embodiment of the present disclosure.

FIG. 3P shows the percent change from baseline (PCFB) over time (hours) of CD3+CD4+CCR7+CD45RA⁻ population with 0.01 mL/kg of Composition A, according to an exemplary embodiment of the present disclosure.

FIGS. 4A-4L show QAD SC injection of saline (placebo control), mid-dose (0.01 mL/kg) or high-dose (0.02 mL/kg) Composition A (n=4/group), as described in Example 1, according to an exemplary embodiment of the present disclosure.

FIG. 4A shows CD45^{int}CD34+ stem cells in peripheral blood on day 1 following a single dose SC injection of saline or Composition A, as described in Example 1, according to an exemplary embodiment of the present disclosure.

FIG. 4B shows CD45^{int}CD34+CD90+ stem cells in peripheral blood on day 1 following a single dose SC injection of saline or Composition A, as described in Example 1, according to an exemplary embodiment of the present disclosure.

FIG. 4C shows CD45^{int}CD34+CD133+ stem cells in peripheral blood on day 1 following a single dose SC injection of saline or Composition A, as described in Example 1, according to an exemplary embodiment of the present disclosure.

FIG. 4D shows CD45^{int}CD34+CD133+CD31+ stem cells in peripheral blood on day 1 following a single dose SC injection of saline or Composition A, as described in Example 1, according to an exemplary embodiment of the present disclosure.

FIG. 4E shows CD45^{int}CD34+CD133+VEGFR2+ stem cells in peripheral blood on day 1 following a single dose SC injection of saline or Composition A, as described in Example 1, according to an exemplary embodiment of the present disclosure.

FIG. 4F shows CD45^{int}CD34+SSEA3+ stem cells in peripheral blood on day 1 following a single dose SC injection of saline or Composition A, as described in Example 1, according to an exemplary embodiment of the present disclosure.

FIG. 4G shows CD45^{int}CD34+ stem cells in peripheral blood on day 5 following the third dose. Each value represents the mean±SD, as described in Example 1, according to an exemplary embodiment of the present disclosure.

FIG. 4H shows CD45^{int}CD34+CD90+ stem cells in peripheral blood on day 5 following the third dose. Each value represents the mean±SD, as described in Example 1, according to an exemplary embodiment of the present disclosure.

FIG. 4I shows CD45^{int}CD34+CD133+ stem cells in peripheral blood on day 5 following the third dose. Each value represents the mean±SD, as described in Example 1, according to an exemplary embodiment of the present disclosure.

FIG. 4J shows CD45^{int}CD34+CD133+CD31+ stem cells in peripheral blood on day 5 following the third dose. Each value represents the mean±SD, as described in Example 1, according to an exemplary embodiment of the present disclosure.

FIG. 4K shows CD45^{int}CD34+CD133+VEGDR2+ stem cells in peripheral blood on day 5 following the third dose. Each value represents the mean±SD, as described in Example 1, according to an exemplary embodiment of the present disclosure.

FIG. 4L shows CD45^{int}CD34+SSEA3+ stem cells in peripheral blood on day 5 following the third dose. Each value represents the mean±SD, as described in Example 1, according to an exemplary embodiment of the present disclosure.

FIG. 5A shows gene set enrichment analysis for interpreting gene expression profiles revealed 17 down-regulated pathways at 8 hours following administration of 0.01 mL/kg Composition A, as described in Example 1, according to an exemplary embodiment of the present disclosure.

FIG. 5B shows gene set enrichment analysis for interpreting gene expression profiles revealed 24 down-regulated pathways at 12 hours following administration of 0.01 mL/kg Composition A, as described in Example 1, according to an exemplary embodiment of the present disclosure.

FIG. 5C shows gene set enrichment analysis for interpreting gene expression profiles revealed 19 down-regulated pathways at 24 hours following administration of 0.01 mL/kg Composition A, as described in Example 1, according to an exemplary embodiment of the present disclosure.

FIG. 5D is a Venn diagram analyses of molecular pathways down-regulated by Composition A (0.01 mL/kg) at 8, 12 and 24 hours post injection compared to pre-dose, as described in Example 1, according to an exemplary embodiment of the present disclosure.

FIG. 5E shows gene set enrichment analysis showed 4 down-regulated pathways at 8 hours, and 1 upregulated pathway at 8 hours following administration of 0.02 mL/kg Composition A, as described in Example 1, according to an exemplary embodiment of the present disclosure.

FIG. 5F shows gene set enrichment analysis showed 9 down-regulated pathways at 12 hours following administration of 0.02 mL/kg Composition A, as described in Example 1, according to an exemplary embodiment of the present disclosure.

FIG. 5G shows gene set enrichment analysis showed 5 down-regulated pathways at 24 hours, and 1 upregulated pathway at 24 hours following administration of 0.02 mL/kg Composition A, as described in Example 1, according to an exemplary embodiment of the present disclosure.

FIG. 5H shows gene set enrichment analysis showed 8 down-regulated pathways at 48 hours, and 1 upregulated pathway at 48 hours following administration of 0.02 mL/kg Composition A, as described in Example 1, according to an exemplary embodiment of the present disclosure.

FIG. 5I is a Venn diagram analyses of molecular pathways down-regulated and up-regulated by Composition A (0.02 mL/kg) at 8, 12, 24 and 48 hours post injection compared to pre-dose, as described in Example 1, according to an exemplary embodiment of the present disclosure.

FIG. 6A is a Venn diagram analyses of molecular pathways down-regulated by Composition A at 8, 12 and 24 hours post injection compared to pre-dose (0.01 mL/kg Composition A), as described in Example 1, according to an exemplary embodiment of the present disclosure.

FIG. 6B is a Venn diagram analyses of molecular pathways down-regulated by Composition A at 8, 12 and 24 hours post injection compared to pre-dose (02 mL/kg Composition A), as described in Example 1, according to an exemplary embodiment of the present disclosure.

FIG. 7A is a Venn diagram analysis of common down-regulated pathways between Composition A vs Placebo and Composition A vs Pre-dose (0.01 ml/kg Composition A), as described in Example 1, according to an exemplary embodiment of the present disclosure.

FIG. 7B is a Venn diagram analysis of common down-regulated pathways between Composition A vs Placebo and Composition A vs Pre-dose (0.02 mL/kg Composition A), as described in Example 1, according to an exemplary embodiment of the present disclosure.

FIG. 8 is a diagram showing the relationship between ALS pathology and down-regulation of pathways and mobilization and activation of stem cells and macrophages by Composition A.

### DETAILED DESCRIPTION OF THE DISCLOSURE

It is understood that the present disclosure is not limited to the particular methods and components, etc., described herein, as these may vary. It is also to be understood that the terminology used herein is used for the purpose of describing particular embodiments only, and is not intended to limit the scope of the present disclosure. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth. Specific methods, devices, and materials are described, although any methods and materials similar or equivalent to those described herein can be used in the practice or testing of the present disclosure.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present disclosure.

“Agent” or “active ingredient” refers to any materials that may be used as or in pharmaceutical compositions that can generate a pharmaceutical effect, for example compounds such as small synthetic or naturally derived organic compounds, nucleic acids, polypeptides, antibodies, fragments, isoforms, variants, or other materials that may be used independently for such purposes, all in accordance with the present disclosure.

“Antagonist” refers to an agent that suppresses or inhibits at least one bioactivity, for example of a protein, cell or physiologic system. An antagonist can be a compound which inhibits or decreases the interaction between a protein or cellular receptor and another molecule, e.g., a target peptide or enzyme substrate. An antagonist may also be a compound that down-regulates expression of a gene or which reduces the amount of expressed protein related to the bioactivity to be antagonized.

“Hematopoiesis” refers to the highly orchestrated process of blood cell development and homeostasis. Prenatally, hematopoiesis occurs in the yolk sack, then liver, and eventually the bone marrow. In normal adults it occurs in bone marrow and lymphatic tissues. All blood cells develop from pluripotent stem cells. Pluripotent cells differentiate into stem cells that are committed to three, two or one hematopoietic differentiation pathways.

The term “immunosuppressive agent” is used interchangeably with “immunosuppressant agent” and refers to an agent that inhibits, slows or reverses the activity of the immune system. In this disclosure, reference to a low dose immunosuppressive agent means a dose low enough such that the immunosuppressive activity is reduced or eliminated.

The terms “stem cells” and “hematopoietic stem cells” are used interchangeably herein. Stem cells can be distinguished from other cell types by two important characteristics. First, stem cells are unspecialized cells capable of renewing themselves through cell division, sometimes after long periods of inactivity. Second, under certain physiologic or experimental conditions, stem cells can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide and differentiate under special conditions. As used herein, the term “stem cells” can refer to multipotent or pluripotent stem cells that are capable of differentiating into all blood cells including erythrocytes, leukocytes and platelets. For instance, the “hematopoictic stem cells” or “stem cells” as used in the disclosure are contained not only in bone marrow but also in umbilical cord blood derived cells.

The term “endogenous stem cells” means stem cells derived from the same individual which is being treated. As used herein, “endogenous stem cells” can be removed from the subject and reimplanted, or can remain in the subject throughout the course of treatment. The term “autochthonous stem cells” means stem cells which are native to the subject being treated, and generally indicates that the stem cells remain in the subject the course of treatment. It is understood, when stem cell mobilizers are administered to a subject according to the methods described herein, that endogenous/autochthonous stem cells are mobilized.

A “stem cell mobilizer,” “mobilizer of hematopoietic stem cells or progenitor cells” or “mobilize” used with respect to stem cells refers to any compound, for example a small organic molecule, synthetic or naturally derived compound, a polypeptide or protein, such as a growth factor or colony stimulating factor or an active fragment or mimic thereof, a nucleic acid, a carbohydrate, an antibody, or any other agent that acts to enhance the migration of stem cells from the bone marrow into the peripheral blood. A stem cell mobilizer can increase the number of hematopoictic stem cells or hematopoictic progenitor/precursor cells in the peripheral blood, thus allowing for a more accessible source of stem cells for use in treating subjects according to the present methods, for example organ transplant recipients, burn victims, those with autoimmune or inflammatory diseases such as IBD, those nerve injury and/or degeneration (including spinal cord injury) or those in need of promoting wound healing, including wounds associated with diabetes. In some embodiments, a stem cell mobilizer refers to any agent that mobilizes CD34+ and/or CD133+ stem cells. In other embodiments, a stem cell mobilizer disrupts CXCL12 (SDF-1)-mediated chemoattraction of CXCR4-expressing cells.

The terms “patient,” “subject,” or “host” are used interchangeably herein, and refer to any individual human or animal to be treated by the present methods, for example a human or non-human primate, bovine, ovine, porcine, feline, canine or rodent.

As used herein, the terms “treatment,” “treating,” “treat” and the like, refer to obtaining a desired pharmacologic or physiologic effect. The pharmacologic and/or physiologic effect can be prophylactic, for example by completely or partially delaying or preventing a particular outcome relating to a disease or disorder, or a symptom thereof, or may be therapeutic, for example by ameliorating or causing a partial or complete cure for a disease or disorder/or symptom or adverse effect thereof.

The present disclosure provides pharmaceutical compositions comprising at least one stem cell mobilizer and at least one immunosuppressive agent or non-immunosuppressive FK binding protein ligand. Suitable stem cell mobilizers are known in the art, and include small organic molecules, polypeptides, nucleic acids, and carbohydrates.

Suitable polypeptide stem cell mobilizers can comprise a cytokine, a colony stimulating factor, a protease or a chemokine. In some embodiments, the cytokine stem cell mobilizers include interleukin-1 (IL-1), interleukin-3 (IL-3), interleukin-6 (IL-6), interleukin-11 (IL-11), interleukin-7 (IL-7), and interleukin-12 (IL12).

Suitable colony stimulating factor stem cell mobilizers can comprise granulocyte colony stimulating factor (G-CSF), granulocyte-macrophage colony stimulating factor (GM-CSF), macrophage colony stimulating factor (M-CSF), stem cell factor, FLT-3 ligand or combinations thereof.

Suitable protease stem cell mobilizers can comprise metalloproteinase (like MMP2 or MMP9) a serine protease, (like cathepsin G, or elastase) a cysteine protease (like cathepsin K) and a dipeptidyl peptidase-1 (DDP-1 OR CD26).

Suitable chemokine stem cell mobilizers can comprise CXCL12, IL-8, Mip-ia, and Gro3.

Suitable nucleic acid stem cell mobilizers can comprise a DNA or an RNA molecule, for example a small interfering RNA (siRNA) molecule or an antisense molecule.

Suitable carbohydrate stem cell mobilizers can comprise a sulfated carbohydrate, for example Fucoidan or sulfated dextran. Fucoidan is a carbohydrate consisting of L-fucose, sulfate and acetate in a molar proportion of 1:1.23:0.36 and can be isolated from the Pacific brown seaweed Fucus evanescens. See Bilan et al., 337 (8) Carbohydrate Research 719-30 (2002). Sulfated dextrans refer to a series of polysaccharides that have variable sulfated patterns and Pomin et al., 15 (12) Glycobiology 1376-1385 (2005); Melo et al., 279 (2) J. Biol. Chem. 20824-20835 (2004); and Farias et al., 275 (38) J. Biol. Chem. 29299-29307 (2000), the entire disclosures of which are herein incorporated by reference.

Other suitable stem cell mobilizers include AMD3100; stromal cell-derived factor (SDF-1); SDF-1 analogs (e.g., CTCE-0214 available for example from Chemokine Therapeutics Corp.); anti-SDF-1 antibodies; cyclophosphamide; stem cell factor (SCF); filgrastim; ancestim; Myeloid Progenitor Inhibitory Factor-1 (MPIF-1), as disclosed in, e.g., U.S. Patent Publication No. 20080274109, the entire disclosure of which are herein incorporated by reference; and Very Late Antigen (VLA-4) antagonists such as an alpha-4 integrin antagonist like Natalizumab or Anti-phospho-Integrin ct4 (Ser988), clone 6.33 (Upstate Cell Signaling Solutions), or a peptide (e.g., phenylacetyl-leu-asp-phe-D-prolincamide available, for example, from Cytel Corp., San Diego Calif.).

In certain embodiments, the stem cell mobilizer comprises a CXCR4 antagonist. In some embodiments, the CXCR4 antagonist is TG-0054 (Burixafor; Phosphonic acid, p-(2-(4-(6-amino-2-(((trans-4-(((3-(cyclohexylamino) propyl) amino) methyl)-cyclohexyl)methyl) amino)-4-pyrimidinyl)-1-piperazinyl) ethyl)-) (TaiGen Biotechnology Co., Ltd. (Taipei, Taiwan)). In other embodiments, the CXCR4 antagonist is AMD3465 (N-(pyridin-2-ylmethyl)-1-[4-(1,4,8,11-tetrazacyclotetradec-1-ylmethyl)phenyl]methanamine). In yet other embodiments, the CXCR4 antagonist is AMD3100. AMD3100, also known as (1,1′-[1,4-phenylenebis(methylene)]bis-1,4,8,11-tetraazacyclo-tetradecane, is a symmetric bicyclam, prototype non-peptide antagonist of the CXCR4 chemokine receptor described, for example, in U.S. Pat. Nos. 6,835,731 and 6,825,351, the entire disclosures of which are herein incorporated by reference. The term “AMD3100” is used interchangeably herein with Plerixafor, rINN, JM3100, and the trade name, Mozobil™. The present disclosure also contemplates using mimetics of AMD3100 in the present pharmaceutical compositions. For example, mutational substitutions at 16 positions located in TM-III, -IV, -V, -VI, and -VII lining the main ligand-binding pocket of the CXCR4 receptor have identified three amino acid residues as the main interaction points for AMD3100; namely Asp¹⁷¹ (AspIV:20), Asp²⁶² (AspVI:23), and Glu²⁸⁸ (GluVII:06). Molecular modeling suggests that one cyclam ring of AMD3100 interacts with Asp¹⁷¹ in TM-IV, whereas the other ring is sandwiched between the carboxylic acid groups of Asp²⁶² and Glu²⁸⁸ from TM-VI and -VII, respectively. In one study, it was found that introduction of only a Glu at position VII:06 and the removal of a neutralizing Lys residue at position VII:02 resulted in a 1000-fold increase in affinity of AMD3100 to within 10-fold of its affinity in CXCR4. Any other suitable AMD3100 mimetic can be used, such as for example, peptide or non-peptide antagonists with improved oral bioavailability which are designed to efficiently and selectively block the CXCR4 receptor.

In other embodiments, the stem cell mobilizer is BKT140 (Biokin Therapeutics, Ltd. (Rchovot, Israel). BKT140, also known as 4F-benzoyl-TN14003, binds to and inhibits the CXCR4 chomokin receptor with high affinity, with an IC₅₀ of ˜1 nmol/L compared with the values obtained with AMD3100. Moreover, BKT140 hinders the cell migration stimulated by CXCL12 within IC₅₀ values of 0.5 to 2.5 nmol/L, compared with IC₅₀ value of 51±17 nmol/L for Plerixafor, suggesting a high mobilization capacity. See, e.g., Peled et al., 20 Clin Cancer Res. 469-79 (2013), the entire disclosure of which is herein incorporated by reference.

As discussed above, the pharmaceutical compositions of the disclosure can comprise at least one immunosuppressive agent with the at least one stem cell mobilizer.

Any suitable immunosuppressive agent can be used in the present pharmaceutical compositions, including: a calcincurin inhibitor (e.g., cyclosporin (CsA) and analogs thereof, ISA (TX) 247, and Tacrolimus); azathioprinc (AZ); mycophenolate mofetil (MMF); mizoribine (MZ); leflunomide (LEF); adrenocortical steroids (also known as adrenocortical hormones, corticosteroids, or corticoids) such as prednisolone and methylprednisolone; sirolimus (also known as rapamycin); everolimus; FK778; TAFA-93; deoxyspergualin (DSG); and 2-amino-2-[2-(4-octylphenyl) ethyl]-1,3-propanediol hydrochloride (FTY720).

Other suitable immunosuppressive agents include: cyclophosphamide; 15-deoxyspergualin (Gusperimus); interferons; sulfasalazine; mimoribine; misoprostol; anti-IL-2 receptor antibodies; thalidomide; anti-tumor necrosis factor antibodies; anti-CD2 antibodies; anti-CD147 antibodies; anti-CD4 antibodies; anti-CD8 antibodies and anti-thymocyte globulin antibodies; ORTHOCLONE® (also known as OKT3, from Ortho Biotech, Raritan, N.J.); SANDIMMUNE® ORAL (cyclosporine), available for example from Sandoz Pharmaceuticals, Hanover, N.J.; PROGRAF®, also known as Tacrolimus, available for example from Fujisawa Pharmaceuticals, Deerfield, Ill.); CELLCEPT®, also known as mycophenolate, available for example from Roche Pharmaceuticals, Nutley, N.J.; and RAPAMUNE®, also known as sirolimus, available for example from Pfizer, Inc, Collegeville, Pa.). In some embodiments, the immunosuppressive agent is rapamycin, Tacrolimus, mycophenolic acid, azathioprinc or cyclophosphamide. Still other suitable immunosuppressive agents include an interleukin-2 alpha-chain blocker (e.g., basiliximab and daclizumab); an inhibitor of inosine monophosphate dehydrogenase (e.g., mycophenolatc mofetil); or an inhibitor of dihydrofolic acid reductase (e.g., methotrexate).

In certain embodiments, the immunosuppressive agent is Tacrolimus. Tacrolimus (also known as FK-506 or Fujimycin) is an immunosuppressive drug that is mainly used after allogeneic organ transplant to reduce the activity of the patient's immune system, and so lower the risk of organ rejection. It reduces interleukin-2 (IL-2) production by T-cells. Tacrolimus is also used in a topical preparation for the treatment of severe atopic dermatitis (eczema), severe refractory uveitis after bone marrow transplants, and the skin condition vitiligo. Tacrolimus is a 23-membered macrolide lactone discovered in 1984 from the fermentation broth of a Japanese soil sample that contained the bacteria Streptomyces tsukubaensis. The drug is sold under the trade names Prograf® given twice daily (intravenous); Advagraf®, which is a sustained release formulation allowing once daily dosing (oral); and Protopic®, which is a topical formulation.

The pharmaceutical compositions of the disclosure can also comprise at least one FK binding protein ligand with at least one stem cell mobilizer. Examples include FK-506 (Tacrolimus) and derivatives/analogs thereof, including 506BD and L0685,818; rapamycin and derivatives/analogs thereof including Way-124466, RAD001, CCI-779, and AP23573; ascomycin and derivatives/analogs thereof including pimecrolimus. Sec, e.g., Liu et al., 23 (11) EXPERT OPIN. THER. PATENTS 1435-49 (2013), the entire disclosure of which is herein incorporated by reference. Furthermore, although the immunosuppressive agent Tacrolimus/FK-506 is an FK binding protein ligand, in certain embodiments, an FK binding protein ligand can comprise a non-immunosuppressive FK binding protein ligand. Examples of non-immunosuppressive ligands include meridamycin, antascomicins, and synthetic ligand of FKBP (SLF).

The present disclosure provides methods for pharmacological mobilization and recruitment of endogenous bone marrow stem cells and immunoregulatory cells comprising administering a pharmaceutical composition disclosed herein. The present disclosure also provides methods for improving wound healing, promoting tissue regeneration and/or preventing allograft rejection. The present disclosure provides methods for immunoregulation and tissue regeneration comprising administering a pharmaceutical composition disclosed herein.

Treatment with the pharmaceutical compositions disclosed herein can facilitate circulation of immune cells including CD4+Foxp3+ and CD8+Foxp3+ regulatory T cells and stem cell populations including CD45^{Int}CD34+ hematopoietic stem cells may increase after administration of the composition. Treatment with the pharmaceutical compositions disclosed herein may down regulate 31 pathways such as the TNFA signaling via NFKB, inflammatory response, apoptosis, allograft rejection and MYC targets VI in mid-dose treated subjects, 14 down-regulated pathways and 2 up-regulated pathways (Interferon-α response and heme metabolism) in high-dose treated subjects compared to the placebo group.

Importantly, the inventors discovered an unexpected and surprising result wherein the synergistic effect of the pharmaceutical composition disclosed here, including for example the AF Combination (defined below), in immunoregulation and tissue repair is not established through calcineurin-dependent immunosuppression but rather because of the ability of low, but not immunosuppressive, dose FK506 to activate the Bone Morphogenic Pathway (BMP) pathway. The pharmaceutical composition disclosed herein can be used in immunoregulatory and regenerative therapy for the treatment of a variety of human diseases related to inflammation and tissue injury, including (without limitation), allograft survival (including liver or kidney allograft survival) without immunosuppression through induction of allograft chimerism that can result in allograft acceptance, accelerated skin wound healing and reduced scar formation in healthy animals and in severely diabetic animals in excisional wound healing, promotion of liver regeneration, prevention of intra-abdominal adhesions following surgery, and inflammatory bowel disease.

The methods disclosed herein can regulate (hyper) inflammation and promote tissue repair/regeneration. The bone marrow is a reservoir of stem cells such as hematopoietic stem cells (HSCs), endothelial progenitor cells (EPCs), mesenchymal stem cells (MSCs), SSEA3+ Muse cells and immunoregulatory cells including Foxp3+ regulatory T cells (Tregs). Methods of the present disclosure can recruit these primordial cells to differentiate at the injured site may regulate inflammation and promote tissue repair.

Accordingly, the present disclosure provides a pharmaceutical composition comprising an effective amount of at least one stem cell mobilizer and at least one immunosuppressive agent or non-immunosuppressive FK binding protein ligand in a low dose (e.g., one-tenth of the normal or immunosuppressive dose). In certain embodiments, the present disclosure further contemplates a pharmaceutical composition comprising a single active agent that has characteristics of both a stem cell mobilizer and an immunosuppressive agent or non-immunosuppressive FK binding protein ligand in a low dose (e.g., one-tenth of the normal or immunosuppressive dose). For example, Tacrolimus can be used as both a stem cell mobilizer and an immunosuppressive agent.

As used herein, an “effective amount” or a “therapeutically effective amount” is used interchangeably and refers to an amount of a pharmaceutical composition of the present disclosure which provides the desired treatment of a subject. As would be appreciated by one of ordinary skill in the art, the therapeutically effective amount of the present pharmaceutical compositions to treat a given disease, disorder or condition will vary from subject to subject, depending on factors such as age, general condition of the subject, the severity of the condition being treated, the particular compound and/or composition administered, and the like. An appropriate therapeutically effective amount of the present pharmaceutical compositions suitable for any individual subject can be readily determined by one of ordinary skill in the art from the information provided herein.

The pharmaceutical compositions of the present disclosure are in biologically compatible form suitable for administration to subjects, for example to humans. The pharmaceutical compositions can further comprise a pharmaceutically acceptable excipient. The term “pharmaceutically acceptable” means suitable for use in humans or animals, for example as approved by a governmental regulatory agency (such as the US Food and Drug Administration) or listed in the U.S. Pharmacopeia or other generally recognized pharmacopeia, or which are generally recognized as safe (GRAS).

As used herein, the term “excipient” refers to a carrier or vehicle (including any suitable diluent, adjuvant or the like) with which the stem cell mobilizer and/or the immunosuppressive agent are administered. Suitable pharmaceutically acceptable excipients can be sterile liquids, such as water and oils, including those of petroleum, animal, vegetable or synthetic origin such as peanut oil, soybean oil, mineral oil, sesame oil and the like. Water can be a pharmaceutically acceptable excipient when the pharmaceutical composition is administered orally. Sterilized water, saline, aqueous dextrose, glycerol, lactated Ringer's solution and the like can be pharmaceutically acceptable excipients when the pharmaceutical composition is injected, such as administered subcutaneously, intramuscularly, or intravascularly (for example intravenously).

Other suitable pharmaceutically acceptable excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition can also contain minor amounts of wetting or emulsifying agents, or pH buffering agents.

The pharmaceutical compositions of the present disclosure can take any suitable form for administration to a subject, such as a human subject, for example solutions, suspensions, emulsions, tablets, pills, capsules, powders, sustained-release formulations and the like. The present pharmaceutical composition can also, for example, be formulated as a suppository, with traditional pharmaceutical excipients such as triglycerides. Oral pharmaceutical formulations of the disclosure can include standard carriers as pharmaceutical excipients, such as pharmaceutical grades of mannitol, lactose, starch, magnesium stearate, sodium saccharine, cellulose, magnesium carbonate, etc. In a specific embodiment, a pharmaceutical composition of the disclosure comprises an effective amount of a stem cell mobilizer and/or an immunosuppressive agent together with a suitable amount of a pharmaceutically acceptable excipient so as to provide the form for proper administration to the patient, for example by subcutaneous, intramuscular, or intravascular (for example intravenous) administration. For a discussion of the properties of solid and liquid pharmaceutically acceptable excipients which are suitable for use in the present pharmaceutical formulations, see, e.g., the excipients described in the Rowe et al., eds., Handbook of Pharmaceutical Excipients, 7th Edition, London: Pharmaceutical Press, 2012, which is incorporated herein by reference.

The pharmaceutical compositions of the present disclosure can be administered by any suitable route of administration, for example oral, parenteral, subcutaneous, intramuscular, intravenous, intra-arterial, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. In certain embodiments, the routes of administration for the present pharmaceutical compositions are oral administration or by injection, for example by subcutaneous, intramuscular, or intravascular (for example intravenous or intra-arterial) injection. In certain embodiments, the route of administration for the present pharmaceutical compositions is by subcutaneous injection.

In some embodiments, the pharmaceutical compositions of the disclosure comprising a stem cell mobilizer and an immunosuppressive agent in a low dose (e.g., one-tenth of the normal or immunosuppressive dose) can be used alone, e.g., a formulation comprising a stem cell mobilizer and an immunosuppressive agent without any other active ingredient, or in concert with at least one other active ingredient at appropriate dosages of the at least one other active ingredient as are known in the art to achieve a desired treatment, for example as defined by routine testing in order to obtain optimal efficacy while minimizing any potential toxicity.

Suitable therapeutically effective amounts and dosage regimens utilizing a pharmaceutical composition of the disclosure can be selected by the ordinarily skilled clinician in accordance with a variety of factors, including species, age, weight, sex, and overall medical condition of the patient; the condition to be treated and its severity or penetration; the route of administration; the renal and hepatic function of the patient; and the particular pharmaceutical composition employed.

In certain embodiments, the immunosuppressive agent comprising pharmaceutical compositions of the disclosure can be administered in low dose amount. The phrase “low dose” or “low dose amount” of an immunosuppressive agent in the context of the present disclosure (in combination with a stem cell mobilizer) refers to the use of a particular amount of an immunosuppressive drug that is lower than typically used for immunosuppression, for example lower than typically used for immunosuppression in a human. In one embodiment, the low dose amount refers to the use of a particular amount that is lower than typically use for immunosuppression of a human organ transplant recipient that is calculated to prevent rejection). In an embodiment, the dose of immunosuppressive agent is in an amount low enough to reduce or eliminate the immunosuppressive activity.

In certain embodiments a low dose of an immunosuppressive agent, for example Tacrolimus, is less than about ⅕, ⅙, 1/7, ⅛, 1/9, 1/10, 1/11, 1/12, 1/13, 1/14, or less then about 1/15 of a normal dose used for immunosuppression in humans. In certain embodiments, the low dose of an immunosuppressive agent, for example Tacrolimus, is about or less than about 1/10 of the amount used for immunosuppression in humans.

In other embodiments, the low dose of the immunosuppressive agent, for example Tacrolimus, is about or less than ½, ⅓, ¼, ⅕, ⅙, 1/7, ⅛, or about or less than about 1/9 of the amount used for immunosuppression in humans. In further embodiments, the low dose of the immunosuppressive agent, for example Tacrolimus, is about or less than about 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.08, or 0.07 times than the typical amount used for a particular situation in humans to generate immunosuppression.

In specific embodiments, a low dose of an immunosuppressive agent (e.g., Tacrolimus) in humans is about 0.01 mg/kg to about 0.5 mg/kg, about 0.01 mg/kg to 0.5 mg/kg, about 0.01 mg/kg to about 0.45 mg/kg, about 0.01 mg/kg to about 0.4 mg/kg, about 0.01 mg/kg to about 0.35 mg/kg, about 0.06 mg/kg to about 0.45 mg/kg, about 0.07 mg/kg to about 0.4 mg/kg, about 0.08 mg/kg to about 0.35 mg/kg, about 0.09 mg/kg to about 0.3 mg/kg, about 0.1 mg/kg to about 0.25 mg/kg, and so on. In one embodiment, the low dose of Tacrolimus in humans is about 0.01 mg/kg to 0.074 mg/kg.

A normal dose of Tacrolimus for immunosuppression in humans is about 0.1 mg/kg/day-0.3 mg/kg/day (oral) and about 0.01 mg/kg/day-0.05 mg/kg/day (IV). In certain embodiments, a low dose of Tacrolimus in humans is about one tenth of the immunosuppressive dose; e.g., about 0.01 mg/kg/day-0.03 mg/kg/day (oral) and about 0.001 mg/kg/day-0.005 mg/kg/day (IV).

In other embodiments, a low dose of Tacrolimus in humans comprises any amount below about 0.1 mg/kg/day for oral administration. The low dose can comprise any amount below about 0.095, 0.09, 0.085, 0.08, 0.075, 0.07, 0.065, 0.06, 0.055, 0.05, 0.045, 0.04, 0.035, 0.03, 0.029, 0.028, 0.027, 0.026, 0.025, 0.024, 0.023, 0.022, 0.021, 0.020, 0.019, 0.018, 0.017, 0.016, 0.05, 0.014, 0.013, 0.012, 0.011, 0.010, 0.009, 0.008, 0.007, 0.006, 0.005, 0.004, 0.003, 0.002, or 0.001 mg/kg/day.

For intravenous administration, a low dose of Tacrolimus in humans comprises any amount below about 0.01 mg/kg/day. The low dose can comprise any amount below about 0.01, 0.009, 0.008, 0.007, 0.006, 0.005, 0.004, 0.003, 0.002, or 0.001 mg/kg/day.

In further embodiments, a low dose of Tacrolimus in humans may result in a blood concentration range of about 0.01 ng/ml to about 10 ng/ml. The concentration can be less than about 10 ng/ml, 9 ng/ml, 8 ng/ml, 7 ng/ml, 6 ng/ml, 5 ng/ml, 4 ng/ml, 3 ng/ml, 2 ng/ml, 1 ng/ml, 0.9 ng/ml, 0.8 ng/ml, 0.7 ng/ml, 0.6 ng/ml, 0.5 ng/ml, 0.4 ng/ml, 0.3 ng/ml, 0.2 ng/ml, 0.1 ng/ml, 0.09 ng/ml, 0.08 ng/ml, 0.07 ng/ml, 0.06 ng/ml, 0.05 ng/ml, 0.04 ng/ml, 0.03 ng/ml, 0.02 ng/ml or 0.01 ng/ml. In another embodiment, the blood Tacrolimus concentrations after administration to humans are less than about 5 ng/ml. The concentration can range from about 0.01, 0.02, 0.03 0.04 or 0.05 ng/ml to about 1, 2, 3, 4, or 5 ng/ml, for example from about 0. 1-4 ng/ml.

In certain embodiments, the stem cell mobilizer in pharmaceutical compositions of the disclosure is AMD3100. In such embodiments, the pharmaceutical composition can comprise a typical human dose for AMD3100, for example about 0.12-0.24 mg/kg. In some embodiments, for a patient who has 60 kg body weight, the dosage of ADM3100 can be about 0.24 mg/kg/day by subcutaneous injection.

The pharmaceutical compositions of the disclosure can be formulated for substantially simultaneous administration to the subject at a single site. As used herein, “substantially simultaneous administration” means that the stem cell mobilizer and immunosuppressive agent or non-immunosuppressive FK binding ligand comprising the pharmaceutical compositions of the disclosure are delivered to the subject at or about the same time. For example, the stem cell mobilizer and immunosuppressive agent or non-immunosuppressive FK binding ligand can be delivered as a single formulation into a single site on the subject, or as separate formulations, for example by delivery to the same single site on a subject by successive administrations, such as successive subcutaneous or intramuscular injections, wherein the separate formulations occupy substantially the same space within the subject's body at substantially the same time. Routes of administration where the pharmaceutical compositions of the disclosure are ultimately absorbed and distributed systemically, such as orally or intra-rectally, are considered as a “substantially simultaneous administration” when the stem cell mobilizer and immunosuppressive agent or non-immunosuppressive FK binding ligand are delivered in a single formulation or are delivered in separate formulations in succession.

Without wishing to be bound by any specific theory, it is believed that the stem cell mobilizer and immunosuppressive agent or non-immunosuppressive FK binding ligand, when given to a subject by substantially simultaneous administration, are absorbed into the subject's body in a way which stimulates mobilization of stem cells synergistically when compared to a stem cell mobilizer and immunosuppressive agent or non-immunosuppressive FK binding ligand administration either separately in time (whether or not at the same site on a subject) or substantially simultaneously but at different sites on a subject. This unknown and surprising synergistic effect indicates that a single formulation of AMD3100 and Tacrolimus AF was statistically-significantly more effective in mobilizing stem cells than either AMD3100 administered alone, Tacrolimus administered alone, or AMD3100 and Tacrolimus administered separately. This synergistic effect is further demonstrated in subjects treated with a pharmaceutical composition comprising a combination of AMD3100 and Tacrolimus unexpectedly had a faster wound healing time, as compared to subjects receiving AMD3100 and Tacrolimus separately (“A+F”). The synergistic combination of AMD3100 and Tacrolimus was advantageous over the A+F treatment in addition to providing faster healing time, for example in terms of administering significantly less dosages of Tacrolimus (twice as much Tacrolimus was used for the A+F treatment) which may, e.g., further avoid undesirable side effects of immunosuppression, and less overall injections were given to the subjects receiving the AMD3100 and Tacrolimus combination treatment.

Where the pharmaceutical compositions of the disclosure comprise the stem cell mobilizing agent AMD3100 and low dose immunosuppressive drug Tacrolimus (FK-506), in the form of combination, this combination is sometimes referred to herein as “AF” or “AF Combination.”

As described above, AMD3100 (Plerixafor or Mozobil) is a CXCR4 antagonist, which was originally developed as an anti-HIV medicine but found to potently mobilize CD34 and other stem cells from their bone marrow niche. AMD3100 was first approved by the FDA in 2008 for use in multiple myeloma cancer patients, for banking of stem cells prior to myeloablative chemotherapy. Today, AMD3100 is used, often with neupogen (G-CSF), to mobilize hematopoietic stem cells in multiple myeloma cancer patients for banking prior to myeloablative chemotherapy. These mobilized stem cells are subsequently transplanted back to the patient after cancer treatment. Thus the drug AMD3100 is well established to be safe and effective.

As described above, FK506 (Tacrolimus or Prograph) was discovered in 1987 from a type of soil bacterium, Streptomyces tsukubaensis. FK506 reduces peptidyl-prolyl isomerase activity by binding to the immunophilin FKBP12 (FK506 binding protein) creating a new complex. This FKBP12-FK506 complex interacts with and inhibits calcineurin thus inhibiting both T-lymphocyte signal transduction and IL-2 transcription. FK506 was first approved by the FDA in 1994 for use in liver transplantation, and its uses have now been extended to include kidney, heart, small bowel, pancreas, lung, trachea, skin, cornea, bone marrow and limb transplants.

The “AF” pharmaceutical compositions described herein, also called “AF Combinations,” thus provide a potent, synergistic activity of AMD3100 and low-dose Tacrolimus in mobilizing, recruiting and retaining of stem cells in the injured sites. As discussed above, the AF Combinations surprisingly show a synergistic effect in treating tissue injury as compared to the separate administration of a stem cell mobilizer (such as AMD3100) and an immunosuppressive agent (such as Tacrolimus) or FK protein binding ligand, or the simultaneous administration of a stem cell mobilizer and an immunosuppressive agent or FK protein binding ligand at different sites.

In certain embodiments, the ratio of Tacrolimus to AMD3100 is about 1/10 to 1/100 in the AF Combinations. In other embodiments, an AF Combinations comprise only two active ingredients, wherein the first active ingredient is AMD3100, and the second active ingredient is Tacrolimus, and wherein the composition comprises 10-40 mg of AMD3100 and 0.1 to 4 mg Tacrolimus. In these and other AF Combinations, the Tacrolimus enhances the potency of the AMD3100. The present pharmaceutical compositions, including AF Combinations, can thus be described in terms of a ratio of (a) an immunosuppressive drug or a FKBP ligand (including an immunosuppressive or a non-immunosuppressive FKBP ligand) to (b) a stem cell mobilizer (e.g., a CXCR antagonist). In certain embodiments, this ratio can be about 1/1, 1/2, 1/3, 1/4, 1/5, 1/6, 1/7, 1/8, 1/9, 1/10, 1/11, 1/12, 1/13, 1/14, 1/15, 1/16, 1/17, 1/18, 1/19, 1/20, 1/21, 1/22, 1/23, 1/24, 1/25, 1/26, 1/27, 1/28, 1/29, 1/30, 1/31, 1/32, 1/33, 1/34, 1/35, 1/36, 1/37, 1/38, 1/39, 1/40, 1/41, 1/42, 1/43, 1/44, 1/45, 1/46, 1/47, 1/48, 1/49, 1/50, 1/51, 1/52, 1/53, 1/54, 1/55, 1/56, 1/57, 1/58, 1/59, 1/60, 1/61, 1/62, 1/63, 1/64, 1/65, 1/66, 1/67, 1/68, 1/69, 1/70, 1/71, 1/72, 1/73, 1/74, 1/75, 1/76, 1/77, 1/78, 1/79, 1/80, 1/81, 1/82, 1/83, 1/84, 1/85, 1/86, 1/87, 1/88, 1/89, 1/90, 1/91, 1/92, 1/93, 1/94, 1/95, 1/96, 1/97, 1/98, 1/99, 1/100, or more.

In some embodiments, the pharmaceutical compositions of the disclosure can comprise (a) an immunosuppressive drug or a FKBP ligand (including an immunosuppressive or a non-immunosuppressive FKBP ligand) and (b) a stem cell mobilizer in a ratio range of about 1/10-1/100, 1/10-1/99, 1/10-1/98, 1/10-1/97, 1/10-1/96, 1/10-1/95, 1/10-1/94, 1/10-1/93, 1/10-1/92, 1/10-1/91, 1/10-1/90, 1/10-1/89, 1/10-1/88, 1/10-1/87, 1/10-1/86, 1/10-1/85, 1/10-1/84, 1/10-1/83, 1/10-1/82, 1/10-1/81, 1/10-1/80, 1/10-1/79, 1/10-1/78, 1/10-1/77, 1/10-1/76, 1/10-1/75, 1/10-1/74, 1/10-1/73, 1/10-1/72, 1/10-1/71, 1/10-1/70, 1/10-1/69, 1/10-1/68, 1/10-1/67, 1/10-1/66, 1/10-1/65, 1/10-1/64, 1/10-1/63, 1/10-1/62, 1/10-1/61, 1/10-1/60, 1/10-1/59, 1/10-1/58, 1/10-1/57, 1/10-1/56, 1/10-1/55, 1/10-1/54, 1/10-1/53, 1/10-1/52, 1/10-1/51, 1/10-1/50, 1/10-1/49, 1/10-1/48, 1/10-1/47, 1/10-1/46, 1/10-1/45, 1/10-1/44, 1/10-1/43, 1/10-1/42, 1/10-1/41, 1/10-1/40, 1/10-1/39, 1/10-1/38, 1/10-1/37, 1/10-1/36, 1/10-1/35, 1/10-1/34, 1/10-1/33, 1/10-1/32, 1/10-1/31, 1/10-1/30, 1/10-1/29, 1/10-1/28, 1/10-1/27, 1/10-1/26, 1/10-1/25, 1/10-1/24, 1/10-1/23, 1/10-1/22, 1/10-1/21, 1/10-1/20, 1/10-1/19, 1/10-1/18, 1/10-1/17, 1/10-1/16, 1/10-1/15, 1/10-1/14, 1/10-1/13, 1/10-1/12, or 1/10-1/11.

In other embodiments, the pharmaceutical compositions can comprise (a) an immunosuppressive drug or a FKBP ligand (including an immunosuppressive or a non-immunosuppressive FKBP ligand) and (b) a stem cell mobilizer in a ratio range of about 1/15-1/100, 1/15-1/99, 1/15-1/98, 1/15-1/97, 1/15-1/96, 1/15-1/95, 1/15-1/94, 1/15-1/93, 1/15-1/92, 1/15-1/91, 1/15-1/90, 1/15-1/89, 1/15-1/88, 1/15-1/87, 1/15-1/86, 1/15-1/85, 1/15-1/84, 1/15-1/83, 1/15-1/82, 1/15-1/81, 1/15-1/80, 1/15-1/79, 1/15-1/78, 1/15-1/77, 1/15-1/76, 1/15-1/75, 1/15-1/74, 1/15-1/73, 1/15-1/72, 1/15-1/71, 1/15-1/70, 1/15-1/69, 1/15-1/68, 1/15-1/67, 1/15-1/66, 1/15-1/65, 1/15-1/64, 1/15-1/63, 1/15-1/62, 1/15-1/61, 1/15-1/60, 1/15-1/59, 1/15-1/58, 1/15-1/57, 1/15-1/56, 1/15-1/55, 1/15-1/54, 1/15-1/53, 1/15-1/52, 1/15-1/51, 1/15-1/50, 1/15-1/49, 1/15-1/48, 1/15-1/47, 1/15-1/46, 1/15-1/45, 1/15-1/44, 1/15-1/43, 1/15-1/42, 1/15-1/41, 1/15-1/40, 1/15-1/39, 1/15-1/38, 1/15-1/37, 1/15-1/36, 1/15-1/35, 1/15-1/34, 1/15-1/33, 1/15-1/32, 1/15-1/31, 1/15-1/30, 1/15-1/29, 1/15-1/28, 1/15-1/27, 1/15-1/26, 1/15-1/25, 1/15-1/24, 1/15-1/23, 1/15-1/22, 1/15-1/21, 1/15-1/20, 1/15-1/19, 1/15-1/18, 1/15-1/17, or 1/15-1/16.

In some embodiments, the ratio range of (a) an immunosuppressive drug or a FKBP ligand (including an immunosuppressive or a non-immunosuppressive FKBP ligand) to (b) a stem cell mobilizer within a pharmaceutical composition of the disclosure can comprise about 1/20-1/100, 1/20-1/99, 1/20-1/98, 1/20-1/97, 1/20-1/96, 1/20-1/95, 1/20-1/94, 1/20-1/93, 1/20-1/92, 1/20-1/91, 1/20-1/90, 1/20-1/89, 1/20-1/88, 1/20-1/87, 1/20-1/86, 1/20-1/85, 1/20-1/84, 1/20-1/83, 1/20-1/82, 1/20-1/81, 1/20-1/80, 1/20-1/79, 1/20-1/78, 1/20-1/77, 1/20-1/76, 1/20-1/75, 1/20-1/74, 1/20-1/73, 1/20-1/72, 1/20-1/71, 1/20-1/70, 1/20-1/69, 1/20-1/68, 1/20-1/67, 1/20-1/66, 1/20-1/65, 1/20-1/64, 1/20-1/63, 1/20-1/62, 1/20-1/61, 1/20-1/60, 1/20-1/59, 1/20-1/58, 1/20-1/57, 1/20-1/56, 1/20-1/55, 1/20-1/54, 1/20-1/53, 1/20-1/52, 1/20-1/51, 1/20-1/50, 1/20-1/49, 1/20-1/48, 1/20-1/47, 1/20-1/46, 1/20-1/45, 1/20-1/44, 1/20-1/43, 1/20-1/42, 1/20-1/41, 1/20-1/40, 1/20-1/39, 1/20-1/38, 1/20-1/37, 1/20-1/36, 1/20-1/35, 1/20-1/34, 1/20-1/33, 1/20-1/32, 1/20-1/31, 1/20-1/30, 1/20-1/29, 1/20-1/28, 1/20-1/27, 1/20-1/26, 1/20-1/25, 1/20-1/24, 1/20-1/23, 1/20-1/22, or 1/20-1/21.

In other embodiments, the ratio range of (a) an immunosuppressive drug or a FKBP ligand (including an immunosuppressive or a non-immunosuppressive FKBP ligand) to (b) a stem cell mobilizer within a pharmaceutical composition of the disclosure can comprise about 1/30-1/100, 1/30-1/99, 1/30-1/98, 1/30-1/97, 1/30-1/96, 1/30-1/95, 1/30-1/94, 1/30-1/93, 1/30-1/92, 1/30-1/91, 1/30-1/90, 1/30-1/89, 1/30-1/88, 1/30-1/87, 1/30-1/86, 1/30-1/85, 1/30-1/84, 1/30-1/83, 1/30-1/82, 1/30-1/81, 1/30-1/80, 1/30-1/79, 1/30-1/78, 1/30-1/77, 1/30-1/76, 1/30-1/75, 1/30-1/74, 1/30-1/73, 1/30-1/72, 1/30-1/71, 1/30-1/70, 1/30-1/69, 1/30-1/68, 1/30-1/67, 1/30-1/66, 1/30-1/65, 1/30-1/64, 1/30-1/63, 1/30-1/62, 1/30-1/61, 1/30-1/60, 1/30-1/59, 1/30-1/58, 1/30-1/57, 1/30-1/56, 1/30-1/55, 1/30-1/54, 1/30-1/53, 1/30-1/52, 1/30-1/51, 1/30-1/50, 1/30-1/49, 1/30-1/48, 1/30-1/47, 1/30-1/46, 1/30-1/45, 1/30-1/44, 1/30-1/43, 1/30-1/42, 1/30-1/41, 1/30-1/40, 1/30-1/39, 1/30-1/38, 1/30-1/37, 1/30-1/36, 1/30-1/35, 1/30-1/34, 1/30-1/33, 1/30-1/32, or 1/30-1/31.

In further embodiments, the pharmaceutical compositions of the disclosure can comprise (a) an immunosuppressive drug or a FKBP ligand (including an immunosuppressive or a non-immunosuppressive FKBP ligand) and (b) a stem cell mobilizer in a ratio range of about 1/40-1/100, 1/40-1/99, 1/40-1/98, 1/40-1/97, 1/40-1/96, 1/40-1/95, 1/40-1/94, 1/40-1/93, 1/40-1/92, 1/40-1/91, 1/40-1/90, 1/40-1/89, 1/40-1/88, 1/40-1/87, 1/40-1/86, 1/40-1/85, 1/40-1/84, 1/40-1/83, 1/40-1/82, 1/40-1/81, 1/40-1/80, 1/40-1/79, 1/40-1/78, 1/40-1/77, 1/40-1/76, 1/40-1/75, 1/40-1/74, 1/40-1/73, 1/40-1/72, 1/40-1/71, 1/40-1/70, 1/40-1/69, 1/40-1/68, 1/40-1/67, 1/40-1/66, 1/40-1/65, 1/40-1/64, 1/40-1/63, 1/40-1/62, 1/40-1/61, 1/40-1/60, 1/40-1/59, 1/40-1/58, 1/40-1/57, 1/40-1/56, 1/40-1/55, 1/40-1/54, 1/40-1/53, 1/40-1/52, 1/40-1/51, 1/40-1/50, 1/40-1/49, 1/40-1/48, 1/40-1/47, 1/40-1/46, 1/40-1/45, 1/40-1/44, 1/40-1/43, 1/40-1/42, or 1/40-1/41.

In other embodiments, the pharmaceutical compositions of the disclosure can comprise (a) an immunosuppressive drug or a FKBP ligand (including an immunosuppressive or a non-immunosuppressive FKBP ligand) and a stem cell mobilizer in a ratio range of about 1/50-1/100, 1/50-1/99, 1/50-1/98, 1/50-1/97, 1/50-1/96, 1/50-1/95, 1/50-1/94, 1/50-1/93, 1/50-1/92, 1/50-1/91, 1/50-1/90, 1/50-1/89, 1/50-1/88, 1/50-1/87, 1/50-1/86, 1/50-1/85, 1/50-1/84, 1/50-1/83, 1/50-1/82, 1/50-1/81, 1/50-1/80, 1/50-1/79, 1/50-1/78, 1/50-1/77, 1/50-1/76, 1/50-1/75, 1/50-1/74, 1/50-1/73, 1/50-1/72, 1/50-1/71, 1/50-1/70, 1/50-1/69, 1/50-1/68, 1/50-1/67, 1/50-1/66, 1/50-1/65, 1/50-1/64, 1/50-1/63, 1/50-1/62, 1/50-1/61, 1/50-1/60, 1/50-1/59, 1/50-1/58, 1/50-1/57, 1/50-1/56, 1/50-1/55, 1/50-1/54, 1/50-1/53, 1/50-1/52, 1/50-1/51, 1/60-1/100, 1/60-1/99, 1/60-1/98, 1/60-1/97, 1/60-1/96, 1/60-1/95, 1/60-1/94, 1/60-1/93, 1/60-1/92, 1/60-1/91, 1/60-1/90, 1/60-1/89, 1/60-1/88, 1/60-1/87, 1/60-1/86, 1/60-1/85, 1/60-1/84, 1/60-1/83, 1/60-1/82, 1/60-1/81, 1/60-1/80, 1/60-1/79, 1/60-1/78, 1/60-1/77, 1/60-1/76, 1/60-1/75, 1/60-1/74, 1/60-1/73, 1/60-1/72, 1/60-1/71, 1/60-1/70, 1/60-1/69, 1/60-1/68, 1/60-1/67, 1/60-1/66, 1/60-1/65, 1/60-1/64, 1/60-1/63, 1/60-1/62, 1/60-1/61,

In other embodiments, the ratio range of (a) an immunosuppressive drug or a FKBP ligand (including an immunosuppressive or a non-immunosuppressive FKBP ligand) to (b) a stem cell mobilizer within a pharmaceutical composition of the disclosure can comprise about 1/70-1/100, 1/70-1/99, 1/70-1/98, 1/70-1/97, 1/70-1/96, 1/70-1/95, 1/70-1/94, 1/70-1/93, 1/70-1/92, 1/70-1/91, 1/70-1/90, 1/70-1/89, 1/70-1/88, 1/70-1/87, 1/70-1/86, 1/70-1/85, 1/70-1/84, 1/70-1/83, 1/70-1/82, 1/70-1/81, 1/70-1/80, 1/70-1/79, 1/70-1/78, 1/70-1/77, 1/70-1/76, 1/70-1/75, 1/70-1/74, 1/70-1/73, 1/70-1/72, 1/70-1/71, 1/80-1/100, 1/80-1/99, 1/80-1/98, 1/80-1/97, 1/80-1/96, 1/80-1/95, 1/80-1/94, 1/80-1/93, 1/80-1/92, 1/80-1/91, 1/80-1/90, 1/80-1/89, 1/80-1/88, 1/80-1/87, 1/80-1/86, 1/80-1/85, 1/80-1/84, 1/80-1/83, 1/80-1/82, 1/80-1/81, 1/90-1/100, 1/90-1/99, 1/90-1/98, 1/90-1/97, 1/90-1/96, 1/90-1/95, 1/90-1/94, 1/90-1/93, 1/90-1/92, or 1/90-1/91.

In certain embodiments, the pharmaceutical compositions of the disclosure comprise (a) a non-immunosuppressive FKBP ligand and (b) a stem cell mobilizer (e.g., a CXCR antagonist). In such embodiments, the ratio of non-immunosuppressive FKBP ligand to stem cell mobilizer can be between about 1/10 to 1/100. In further embodiments, the ratio can be greater than about 1/10 including about 1/1, 1/2, 1/3, 1/4, 1/5, 1/6, 1/7, 1/8 or 1/9. In other embodiments, the ratio can be less than about 1/100 including, but not limited to, about 1/150, 1/200, 1/250, 1/300, 1/350, 1/400, 1/450, and 1/500 or more (including ranges of the foregoing).

In certain embodiments, the pharmaceutical compositions of the disclosure, including AF Combinations, may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

In other embodiments, the present pharmaceutical compositions, including AF Combinations, can be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

In other embodiments, the pharmaceutical compositions of the disclosure, including AF Combinations, can be administered every other day for about 2 days, every other day for about 3 days, every other day for about 4 days, every other day for about 5 days, every other day for about 6 days, every other day for about 7 days, every other day for about 8 days, every other day for about 9 days, every other day for about 10 days, every other day for about 11 days, every other day for about 12 days, every other day for about 13 days, every other day for about 14 days, every other day for about 15 days, every other day for about 16 days, every other day for about 17 days, every other day for about 18 days, every other day for about 19 days, every other day for about 20 days, every other day for about 21 days, every other day for about 22 days, every other day for about 23 days, every other day for about 24 days, every other day for about 25 days, every other day for about 26 days, every other day for about 27 days, every other day for about 28 days, every other day for about 29 days, every other day for about 30 days, or every other day for about 31 days or more.

In other embodiments, the pharmaceutical compositions of the disclosure, including AF Combinations, can be administered about once every day, about once every 2 days (also sometimes stated herein as once every other day), about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days. In certain embodiments, the present pharmaceutical compositions can be administered every other day.

In other embodiments, the pharmaceutical compositions of the disclosure, including AF Combinations, can be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, or about once every 20 weeks.

In other embodiments, the pharmaceutical compositions of the disclosure, including AF Combinations, can be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

In other embodiments, the pharmaceutical compositions of the disclosure, including AF Combinations, can be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

In other embodiments, the pharmaceutical compositions of the disclosure, including AF Combinations, can be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

In certain embodiments, the pharmaceutical compositions of the disclosure, including AF Combinations, can be administered in a dosing regimen or treatment method comprising administering the pharmaceutical composition to a subject who is suffering from a tissue injury, disease or inflammation in one or more doses at the time a tissue injury, disease or inflammation is incurred or observed, and at about one month, about two months and about three months after a tissue injury is incurred or observed, for a total of four administrations. Any suitable administration route can be used. In some embodiments of the dosing regimen or treatment method, the administration route used is subcutaneous or intramuscular. In other embodiments of the dosing regimen or treatment method, the administration route is subcutaneous. In certain embodiments, the one or more doses can be administered about when the tissue injury is incurred or observed and at about one month, about two months and about three months after a tissue injury is incurred or observed can comprise one dose, two doses, three doses, four doses, five doses, six doses, seven doses, eight doses, nine doses or ten doses. The one or more doses can be administered over the course of one or more days from when the tissue injury is incurred or observed and at about one month, about two months and about three months after a tissue injury, for example one dose every day or every other day. The one or more doses can be administered in evenly- or unevenly-spaced intervals from when the tissue injury is incurred or observed and at about one month, about two months and about three months after a tissue injury. In one embodiment, the one or more doses can be administered on about the day the tissue injury was incurred or observed (day zero), and again at about days 2, 4, 6, and 8 after the day the tissue injury was incurred or observed; about on the one-month anniversary of the day the tissue injury was incurred or observed and on about days 2, 4, 6 and 8 after the one-month anniversary of the day the tissue injury was incurred or observed; again on about the two-month anniversary of the day the tissue injury was incurred or observed and on about days 2, 4, 6 and 8 after the two-month anniversary of the day the tissue injury was incurred or observed; and again on about the three-month anniversary of the day the tissue injury was incurred or observed and on about days 2, 4, 6 and 8 after the three-month anniversary of the day the tissue injury was incurred or observed.

In other embodiments, the pharmaceutical compositions of the disclosure, including AF Combinations, can be administered in a dosing regimen or treatment method comprising administering the pharmaceutical composition every other day to a subject who is suffering from a tissue injury. Any suitable administration route can be used, for example subcutaneous or intramuscular administration. In certain embodiments, the administration route is subcutaneous. Dosing of the present pharmaceutical compositions every other day can be continued until the tissue injury has been treated, and/or can be continued for a predetermined period of time, for example about one week, 8 days, 9 days, 10 days, 11 days, 12 days, 13 days, 2 weeks, 3 weeks, 4 weeks, 29 days, 30 days, 5 weeks or six weeks.

In treatment methods of the disclosure, the tissue injury which triggers administration of the first dose of the present pharmaceutical compositions, including the first does of AF Combinations, can be any tissue injury that signals or indicates that a particular treatment is necessary. For example, the tissue injury can be an organ transplant (including liver, heart, lung, kidney or corneal transplant or a skin graft), the occurrence of a burn, wound, nerve injury and/or degeneration (including spinal cord injury), or the diagnosis of IBD or other autoimmune or inflammatory disease or the occurrence of an episode of IBD or other autoimmune or inflammatory disease. For example, administration of the first dose can be immediately upon occurrence of the tissue injury, or as soon thereafter as is practical or medically feasible, for example on the same day that the tissue injury occurred or was observed, such as within about one minute, five minutes, thirty minutes, sixty minutes, ninety minutes, 2 hours, 3 hours, 4 hours, 5 hours, 10 hours, 12 hours or 18 hours after occurrence of the tissue injury. In some embodiments, administration of the first dose can be delayed from the occurrence of the tissue injury, for example by about one day, 2 days, 3 days, 4 days, 5 days, 6 days or 7 days.

Any type of burn or wound can be treated with the pharmaceutical compositions and methods of the disclosure (including AF Combinations), including lacerations, tears, abrasions, punctures or combinations of these. Wounds that can be treated by the pharmaceutical compositions and methods of the disclosure can be generated by any source, such as by physical means (e.g., accident, inflicted by self or others, surgical intervention, etc.) or can be generated as sequelae to a disease, disorder or condition such as diabetes or immobility. Burns that can be treated by the pharmaceutical compositions and methods of the disclosure can be generated by any source, for example by exposure of skin or other tissue to extreme heat or cold.

Large full-thickness burns and soft tissue injuries continue to pose significant surgical and medical challenges in both military and civilian injuries, due to limitations of autogenous skin, wound infection, severe metabolic stress and other associated injuries. Human deceased donor skin allografts represent a suitable and much used temporizing option for skin cover following severe burn injury. However, graft rejection is common once the immune suppressive effect of the burn has subsided, and post-burn scars generally occur. Hypertrophic scarring is also extremely common, and is the source of most morbidity related to burns. The inventor has now discovered that the present pharmaceutical compositions, including AF Combinations, and methods can mobilize populations of autochthonous stem cells and induce host repopulation of skin allografts. This conversion to chimeric skin permits extended graft acceptance, or “take,” without the need for immunosuppression.

In one embodiment, the disclosure provides a method of treating full-thickness burns or soft tissue injuries in a subject, comprising administering a pharmaceutical composition of the disclosure, such as AF Combinations, every other day, for example beginning on the day the burn or wound is incurred for a predetermined period of time or until the burn or wound is substantially healed.

Diabetes affects nearly two hundred million people worldwide, and a great number of diabetics may have decrease wound-healing ability. Among patients with diabetes, 15% may also develop wounds such as a foot ulcer, and 12-24% of individuals with such foot ulcers may require amputation. Healing a diabetic foot ulcer or other wound more quickly can limit the complications that may lead to lower extremity amputation, morbidity and mortality in the diabetic subject. However, diabetic foot ulcers and other wounds suffered by diabetics are generally hard to heal. The inventor has now discovered that the present pharmaceutical compositions, including AF Combinations, and methods can enhance the healing of diabetic wounds, such as foot ulcers (see, e.g., Example 3 below).

In one embodiment, the disclosure provides a method of treating wounds on a diabetic subject, for example diabetic ulcers (including diabetic foot ulcers) comprising administering a pharmaceutical composition of the disclosure, such as AF Combinations, every other day, for example beginning on the day the wound is incurred or the ulcer is observed for a predetermined amount of time or until the wound is substantially healed.

Inflammatory bowel disease (IBD), including ulcerative colitis and Crohn's disease, is a chronic relapsing disease that leads to structural damage with destruction of the bowel wall. These conditions are characterized not only by the sub-mucosal accumulation of inflammatory cells, but also by the severe damage to the epithelial layer. Based on the idea that IBD is initiated and maintained by spontaneous development of mucosal inflammation, current treatment approaches are predominately aimed at suppressing overt inflammation and include the use of pharmacological agents (corticosteroids and immune-modulators), biologics (anti-TNF-alpha), and surgery to remove sections of inflamed bowel. However, these treatment modalities have their limitations, in part due to patient non-adherence and relapse. Moreover, approximately ⅓ of IBD patients do not respond to any given therapy, and there is no cure for IBD. Currently, most IBD therapies in development are antibody-based biologics, which all have the potential for loss of therapeutic response due to the generation of antibodies to the biologics, sometimes called anti-drug antibodies or ADA.

Recent clinical studies have featured “mucosal healing” as the most significant prognostic factor for long-term remission in IBD patients, suggesting that accomplishment of epithelial regeneration is critically required to improve the treatment for IBD. A regenerative medicine approach using cell-based therapies is currently viewed as one of the most promising options for the curative treatment of IBD. Stem cells are the focus of many applications in regenerative medicine because of their extensive ability to self-renew and to generate differentiated progeny cells. Mesenchymal stromal cells (MSCs) are attractive for cell therapy due to their immunomodulatory and regenerative properties and robust in vitro proliferative capacity. Autologous and allogeneic adipose- or bone marrow-derived sources of MSCs have therefore been utilized in the early phase clinical trials for the treatment of IBD. Despite the encouraging results of recent clinical trials employing stem cell-based therapies as treatment for IBD, the complex, time consuming and expensive process needed to harvest, expand and transplant the cells makes it difficult to treat large numbers of patients. The inventor has now discovered that the present pharmaceutical compositions, including AF Combinations, and methods can treat autoimmune or inflammatory diseases such as IBD (including colitis and Crohn's disease) by mobilizing autochthonous stem cells to the site of inflammation and/or damage in the gut (see, e.g., Example 5 below).

In one embodiment, the disclosure provides a method of treating an autoimmune disease or disorder in a subject, for example IBD (including colitis or Crohn's disease), comprising administering a pharmaceutical composition of the disclosure, such as AF Combinations, every other day, for example beginning on the day the autoimmune disease or disorder is diagnosed or symptoms or an episode related to the autoimmune disease or disorder is observed, until the autoimmune disease or disorder is treated, or the symptoms or episode related to the autoimmune disease or disorder, is treated or for a predetermined time, such as three weeks.

The incidence of spinal cord injury (SCI) in the United States is more than 10,000 per year, resulting in 720 per million persons and enduring permanent disability each year. SCI involves impairment in motor and/or sensory function, which is characterized by the rapid development of a necrotic core of damaged tissue at the site of injury, followed by the long delayed secondary degeneration. This secondary degeneration lasts over weeks or months and is accompanied by chronic progressive destruction of resident cells, including oligodendrocytes, and further demyelination of neuropathways. To date, no proven therapeutic modality exists that has demonstrated a positive effect in neurologic outcome for SCI.

Advances in stem cell biology in the last decade have shown that stem cells might provide a good source of neurons and glia, as well as exerting a neuroprotective effect on the host tissue, thus opening new horizons for tissue engineering and regenerative medicine. Despite the encouraging results of recent animal studies and clinical trials employing stem cell-based therapies as treatment for SCI, the recovery is not complete and the facilitated repair of the spinal cord still remains insufficient. Moreover, the complex, time consuming and expensive process needed to harvest, expand and transplant endogenous cells makes it difficult to effectively treat large numbers of patients. The AF combination is attractive for therapy of SCI due to its anti-inflammatory and regenerative properties. The inventor has now unexpectedly discovered that the present pharmaceutical compositions, including AF Combinations, and methods can treat SCI by mobilizing autochthonous stem cells to the site of damage (see, e.g., Example 6 below).

In one embodiment, the disclosure provides a method of treating SCI, including acute injury and secondary degradation of spinal nerves, in a subject, comprising administering a pharmaceutical composition of the disclosure, such as AF Combinations, every other day, for example beginning on the day the SCI is incurred, until the SCI, or the symptoms related to the SCI, is treated, or for a predetermined time, such as 29 or 30 days after occurrence of the SCI. In some embodiments, administration of a pharmaceutical composition of the disclosure, such as AF Combinations, can be delayed for a period of time after occurrence of the SCI, for example by one day or five days after occurrence of the SCI.

Anyone practicing the treatment methods of the disclosure to treat a tissue injury can readily determine whether the administration of the present pharmaceutical compositions is treating the tissue injury using well-known techniques or knowledge. For example, the healing of wounds or burns can be visually observed by periodic monitoring. Treatment of autoimmune diseases or disorders, such as IBD, can be determined by monitoring the subject for changes in the degree of inflammation in the affected regions, the lessening of related symptoms, and/or changes in the levels of biomarkers (such as inflammatory cytokines or autoantibodies) in the blood or in tissues. Treatment of SCI can be determined by monitoring the subject over time for the return of sensation to the affected parts of the body or by observing changes in motor function.

In an embodiment of the present disclosure, methods to mobilize various stem and immune cells in a subject, comprising administering a pharmaceutical composition, are provided. In an embodiment, the pharmaceutical composition comprises a stem cell mobilizer, and an immunosuppressive agent or non-immunosuppressive FK binding protein ligand. In an embodiment, the immunosuppressive agent or non-immunosuppressive FK binding protein ligand is administered at a sub-immunosuppressive dose. In an embodiment, the stem cell mobilizer comprises AMD 3100. In an embodiment, the dose range of the stem cell mobilizer, or AMD3100, is about 0.12 mg/kg to about 0.48 mg/kg. In an embodiment, the dose of the stem cell mobilizer, or AMD3100, is about 0.14 mg/kg, about 0.16 mg/kg, about 0.18 mg/kg, about 0.20 mg/kg, about 0.22 mg/kg, about 0.24 mg/kg, about 0.26 mg/kg, about 0.28 mg/kg, about 0.30 mg/kg, about 0.32 mg/kg, about 0.34 mg/kg, about 0.36 mg/kg, about 0.38 mg/kg, about 0.40 mg/kg, about 0.42 mg/kg, about 0.44 mg/kg, about 0.46 mg/kg, or about 0.48 mg/kg. In an embodiment, the stem cell mobilizer, or AMD3100, plasma concentration range at about 1, about 3, and/or about 8 hours is about 200 ng/ml to about 1,000 ng/mL. In one embodiment, the stem cell mobilizer, or AMD3100, plasma concentration at about 1, about 3, and/or about 8 hours is about 200 ng/mL, about 250 ng/mL, about 300 ng/ml, about 350 ng/mL, about 400 ng/mL, about 450 ng/ml, about 500 ng/ml, about 550 ng/ml, about 600 ng/ml, about 650 ng/ml, about 700 ng/mL, about 750 ng/mL, about 800 ng/ml, about 850 ng/ml, about 900 ng/mL, about 950 ng/ml or about 1,000 ng/mL.

In an embodiment, the immunosuppressive agent or non-immunosuppressive FK binding protein ligand comprises Tacrolimus. In an embodiment, the dose range of the immunosuppressive agent or non-immunosuppressive FK binding protein ligand, or Tacrolimus, is about 0.0025 mg/kg to about 0.01 mg/kg. In an embodiment, the dose of the immunosuppressive agent or non-immunosuppressive FK binding protein ligand, or Tacrolimus, is about 0.0030 mg/kg, about 0.0035 mg/kg, about 0.0040 mg/kg, about 0.0045 mg/kg, about 0.0050 mg/kg, about 0.0055 mg/kg, about 0.0060 mg/kg, about 0.0065 mg/kg, about 0.0070 mg/kg, about 0.0075 mg/kg, about 0.0080 mg/kg, about 0.0085 mg/kg, about 0.0090 mg/kg, about 0.0095 mg/kg, or about 0.01 mg/kg.

In an embodiment, the immunosuppressive agent or non-immunosuppressive FK binding protein ligand, or Tacrolimus, plasma concentration range at about 1, about 3, about 8, about 12 and/or about 24 hours is about 1 ng/mL to about 7 ng/ml. In an embodiment, the immunosuppressive agent or non-immunosuppressive FK binding protein ligand, or Tacrolimus, plasma concentration range at about 1, about 3, about 8, about 12 and/or about 24 hours is about 1 ng/ml, about 2 ng/mL, about 3 ng/ml, about 4 ng/ml, about 5 ng/ml, about 6 ng/ml or about 7 ng/mL.

In an embodiment, the stem cells that are mobilized comprise one or more of CD45^{Int}CD34+ hematopoictic stem cells (HSCs), CD45^{Int}CD34+CD90+ mesenchymal stem cells (MSCs), CD45^{Int}CD34+CD133+HSCs, CD45ICD34+CD133+CD31+ endothelial progenitor cells (EPCs), CD45^{Int}CD34+CD133+VEGFR2+ early EPCs, and CD45^{Int}SSEA3+ multi-lineage differentiating stress enduring cells (Muse cells). In an embodiment, the immune cells including immunoregulator cells that are mobilized comprise one or more of CD3+ T cells, CD3+CD4+ T cells, CD3+CD8+ T cells, CD3+/CD4+/Q3 CCR7+CD45RA-central memory CD4 T-lymphocytes, CD3+/CD8+/Q3 CCR7+CD45RA-central memory CD8 T-lymphocytes, CD3+/CD4+/Q4 CCR7-CD45RA-effector memory CD4 T-lymphocytes, CD3+/CD8+/Q4 CCR7-CD45RA-effector memory CD8 T-lymphocytes, CD19+B cells, CD3+CD4+Foxp3+ regulatory T cells (Tregs), and CD3+CD8+Foxp3+ Tregs.

In an embodiment of the present disclosure, methods to down-regulate molecular pathways for treating one or more of inflammation, allograft rejection and autoimmune disease comprising administering a pharmaceutical composition to a subject are provided. In an embodiment, the pharmaceutical composition comprises a stem cell mobilizer, and an immunosuppressive agent or non-immunosuppressive FK binding protein ligand. In an embodiment, the immunosuppressive agent or non-immunosuppressive FK binding protein ligand is administered at a sub-immunosuppressive dose. In an embodiment, the stem cell mobilizer comprises AMD 3100. In an embodiment, the dose range of the stem cell mobilizer, or AMD3100, is about 0.12 mg/kg to about 0.48 mg/kg. In an embodiment, the dose of the stem cell mobilizer, or AMD3100, is about 0.14 mg/kg, about 0.16 mg/kg, about 0.18 mg/kg, about 0.20 mg/kg, about 0.22 mg/kg, about 0.24 mg/kg, about 0.26 mg/kg, about 0.28 mg/kg, about 0.30 mg/kg, about 0.32 mg/kg, about 0.34 mg/kg, about 0.36 mg/kg, about 0.38 mg/kg, about 0.40 mg/kg, about 0.42 mg/kg, about 0.44 mg/kg, about 0.46 mg/kg, or about 0.48 mg/kg. In an embodiment, the stem cell mobilizer, or AMD3100, plasma concentration range at about 1, about 3, and/or about 8 hours is about 200 ng/ml to about 1,000 ng/mL. In one embodiment, the stem cell mobilizer, or AMD3100, plasma concentration at about 1, about 3, and/or about 8 hours is about 200 ng/ml, about 250 ng/mL, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/mL, about 850 ng/ml, about 900 ng/ml, about 950 ng/ml or about 1,000 ng/mL.

In an embodiment, the immunosuppressive agent or non-immunosuppressive FK binding protein ligand comprises Tacrolimus. In an embodiment, the dose range of the immunosuppressive agent or non-immunosuppressive FK binding protein ligand, or Tacrolimus, is about 0.0025 mg/kg to about 0.01 mg/kg. In an embodiment, the dose of the immunosuppressive agent or non-immunosuppressive FK binding protein ligand, or Tacrolimus, is about 0.0030 mg/kg, about 0.0035 mg/kg, about 0.0040 mg/kg, about 0.0045 mg/kg, about 0.0050 mg/kg, about 0.0055 mg/kg, about 0.0060 mg/kg, about 0.0065 mg/kg, about 0.0070 mg/kg, about 0.0075 mg/kg, about 0.0080 mg/kg, about 0.0085 mg/kg, about 0.0090 mg/kg, about 0.0095 mg/kg, or about 0.01 mg/kg.

In an embodiment, the immunosuppressive agent or non-immunosuppressive FK binding protein ligand, or Tacrolimus, plasma concentration range at about 1, about 3, about 8, about 12 and/or about 24 hours is about 1 ng/mL to about 7 ng/ml. In an embodiment, the immunosuppressive agent or non-immunosuppressive FK binding protein ligand, or Tacrolimus, plasma concentration range at about 1, about 3, about 8, about 12 and/or about 24 hours is about 1 ng/ml, about 2 ng/ml, about 3 ng/ml, about 4 ng/mL, about 5 ng/ml, about 6 ng/ml or about 7 ng/mL.

In an embodiment, the inflammatory and autoimmune disease or disorder comprise one or more of Addison's Disease, Alopecia areata, ARDS, Ankylosing Spondylitis, Antiphospholipid Antibody Syndrome, Autoimmune Encephalitis, Autoimmune Hepatitis, Behcet's Disease, Bullous pemphigoid, Chronic Recurrent Multifocal Osteomyelitis, Diabetes Type I, Diverticulitis, Epidermolysis bullosa acquisita (EBA), Gout, Granulomatosis with polyangiitis, Graves Disease, Guillan-Barre Syndrome, Hashimoto's Thyroiditis, Henoch-Schonlein Purpura, IgA Nephropathy, Idiopathic pulmonary fibrosis, Juvenile Dermatomyositis, Juvenile Idiopathic Arthritis, Juvenile Lupus (SLE), Juvenile Scleroderma, Juvenile Vasculitis, Kawasaki Disease, Lambert-Eaton myasthenic syndrome (LEMS), Lichen planus, Lupus (Systemic Lupus Erythematosus), Meniere's disease, Mixed Connective Tissue Disease, Morvan's syndrome, Multiple Sclerosis, Myasthenia gravis, Myositis, Neuromyelitis Optica, Polymyalgia rheumatica, Poststreptococcal Inflammatory Syndromes, Primary biliary cholangitis, Primary sclerosing cholangitis, Psoriasis, Psoriatic Arthritis, Raynaud's phenomenon, Reactive Arthritis, Rheumatoid Arthritis, Sarcoidosis, Scleritis, Scleroderma, Sjogren's Syndrome, Spondyloarthritis/Spondyloarthropathy, Systemic Juvenile Idiopathic Arthritis, Stiff-person syndrome, Sweet's syndrome, Transverse myelitis, Undifferentiated Connective Tissue Disease, Uveitis, Vasculitis and Vitiligo. In an embodiment, the allograft rejection comprises one or more of liver transplantation, corneal transplantation, intestinal transplantation, uterus transplantation, skin transplantation, kidney transplantation, lung transplantation, heart transplantation, pancreas and/or islet transplantation, bone marrow transplantation and vascularized composite allotransplantation (VCA).

In an embodiment, the down-regulated pathways for treating one or more of inflammation, allograft rejection and autoimmune disease comprise one or more of TNFalpha signaling via NFKB, inflammatory response, allograft rejection, IL-2 STAT5 signaling, IL-6 JAK/STAT3 signaling, oxidative phosphorylation, MTORC1 signaling, Hypoxia, P53, coagulation, apoptosis, complement, interferon gamma response and oxidative oxygen stress (ROS) pathway.

In an embodiment of the present disclosure, methods to down-regulate molecular pathways for one or more of: treating ischemia, reperfusion injury, neurodegenerative disease or disorder, inflammatory pain or disorder and steatohepatitis, anti-thrombosis, anti-aging, anti-fibrosis and anti-cancer, comprising administering to the subject a pharmaceutical composition, are provided. In an embodiment, the pharmaceutical composition comprises a stem cell mobilizer, and an immunosuppressive agent or non-immunosuppressive FK binding protein ligand. In an embodiment, the immunosuppressive agent or non-immunosuppressive FK binding protein ligand is administered at a sub-immunosuppressive dose. In an embodiment, the stem cell mobilizer comprises AMD 3100. In an embodiment, the dose range of the stem cell mobilizer, or AMD3100, is about 0.12 mg/kg to about 0.48 mg/kg. In an embodiment, the dose of the stem cell mobilizer, or AMD3100, is about 0.14 mg/kg, about 0.16 mg/kg, about 0.18 mg/kg, about 0.20 mg/kg, about 0.22 mg/kg, about 0.24 mg/kg, about 0.26 mg/kg, about 0.28 mg/kg, about 0.30 mg/kg, about 0.32 mg/kg, about 0.34 mg/kg, about 0.36 mg/kg, about 0.38 mg/kg, about 0.40 mg/kg, about 0.42 mg/kg, about 0.44 mg/kg, about 0.46 mg/kg, or about 0.48 mg/kg. In an embodiment, the stem cell mobilizer, or AMD3100, plasma concentration range at about 1, about 3, and/or about 8 hours is about 200 ng/mL to about 1,000 ng/ml. In one embodiment, the stem cell mobilizer, or AMD3100, plasma concentration at about 1, about 3, and/or about 8 hours is about 200 ng/mL, about 250 ng/ml, about 300 ng/mL, about 350 ng/ml, about 400 ng/mL, about 450 ng/ml, about 500 ng/ml, about 550 ng/ml, about 600 ng/mL, about 650 ng/mL, about 700 ng/ml, about 750 ng/mL, about 800 ng/ml, about 850 ng/mL, about 900 ng/mL, about 950 ng/mL or about 1,000 ng/mL.

In an embodiment, the immunosuppressive agent or non-immunosuppressive FK binding protein ligand comprises Tacrolimus. In an embodiment, the dose range of the immunosuppressive agent or non-immunosuppressive FK binding protein ligand, or Tacrolimus, is about 0.0025 mg/kg to about 0.01 mg/kg. In an embodiment, the dose of the immunosuppressive agent or non-immunosuppressive FK binding protein ligand, or Tacrolimus, is about 0.0030 mg/kg, about 0.0035 mg/kg, about 0.0040 mg/kg, about 0.0045 mg/kg, about 0.0050 mg/kg, about 0.0055 mg/kg, about 0.0060 mg/kg, about 0.0065 mg/kg, about 0.0070 mg/kg, about 0.0075 mg/kg, about 0.0080 mg/kg, about 0.0085 mg/kg, about 0.0090 mg/kg, about 0.0095 mg/kg, or about 0.01 mg/kg.

In an embodiment, the immunosuppressive agent or non-immunosuppressive FK binding protein ligand, or Tacrolimus, plasma concentration range at about 1, about 3, about 8, about 12 and/or about 24 hours is about 1 ng/mL to about 7 ng/ml. In an embodiment, the immunosuppressive agent or non-immunosuppressive FK binding protein ligand, or Tacrolimus, plasma concentration range at about 1, about 3, about 8, about 12 and/or about 24 hours is about 1 ng/mL, about 2 ng/ml, about 3 ng/ml, about 4 ng/ml, about 5 ng/ml, about 6 ng/mL or about 7 ng/mL.

In an embodiment, the ischemia or reperfusion injury comprise one or more of Decubitus Ulcers (Pressure Sores), Gangrene, Ischemic heart disease (coronary heart disease or coronary artery disease), Hypoxic ischemic encephalopathy (HIE), Interstitial lung disease, Intestinal ischemia and ischemic colitis, Ischemic stroke, Limb ischemia (Peripheral Artery Disease), Myocardial Infarction, Renal Ischemia, Pulmonary infarction, Spinal cord ischemia and Transient Ischemic Attack. In an embodiment, the down-regulated pathways for treating ischemia or reperfusion injury comprise one or more of hypoxia, ROS pathway, P53 pathway, mTORC1 signaling pathway, apoptosis, TNFalpha signaling via NFKB, Inflammatory response, IL-2 STAT5 signaling, IL-6 JAK/STAT3 signaling and Oxidative phosphorylation.

In an embodiment, the anti-thrombotic effect comprises one or more of Deep vein thrombosis, Pulmonary embolism, Peripheral artery disease, Antiphopholipid syndrome, Thromboflebitis, Systemic Lupus Erythematosus and Inflammatory bowel diseases. In an embodiment, the down-regulated pathways for anti-thrombotic effect comprise one or more of Coagulation pathway, Complement pathway, Inflammatory response and ROS pathway.

In an embodiment, the aging comprises one or more of Age-related macular degeneration, Alzheimer's Disease, Atherosclerosis, Cancers, Cataracts, Chronic Kidney Disease, Heart Failure, Huntington's Disease, Mild Cognitive Impairment, Type 2 Diabetes, Osteoarthritis, Osteoporosis, Parkinson's Disease, Presbycusis and Sarcopenia. In an embodiment, the down-regulated pathways for the treatment of aging comprise one or more of mTORC1 signaling, IL-6 JAK/STAT3 signaling, Inflammatory response, P53 pathway, Apoptosis, ROS pathway and Unfolded protein response.

In an embodiment, the fibrosis comprises any fibrosis caused by injury to organs and tissues, the injury comprising one or more of Liver Cirrhosis, Non-Alcoholic Steatohepatitis, Alcoholic Liver disease, Idiopathic Pulmonary fibrosis, Sarcoidosis, Myocardial fibrosis, Chronic Kidney Disease, Diabetic Nephropathy, Keloids, Hypertrophic scars, Chronic pancreatitis, Myelofibrosis, Retinal fibrosis and Retroperitoneal fibrosis. In an embodiment, the down-regulated pathways for treating fibrosis comprise one or more of WNT beta-catenin signaling and TGF-beta signaling.

In an embodiment, the cancer comprises MYC expressing cancers. In an embodiment, the cancer comprises one or more of breast cancer, lung cancer, colorectal cancers, prostate cancer, melanoma and non-melanoma skin cancers, bladder cancer, stomach cancer and liver cancer. In an embodiment, the down-regulated pathways for treating cancer, or MYC expressing cancers, comprise one or more of MYC targets V1, MYC Targets V2, Kras signaling up, Androgen response, Peroxisome, DNA repair, unfolded protein response, ROS pathway, UV response DN, G2M Checkpoint and MTORC1 Signaling.

In an embodiment, the neurodegenerative disease or disorder comprises one or more of Chronic inflammatory demyelinating polyneuropathy (CIDP), dementia-type diseases, demyelinating diseases, frontotemporal dementia, Lewy body dementia, corticobasal degeneration, primary progressive aphasia, spinal muscular atrophy, Kennedy's disease, mitochondrial encephalomyopathy, lactic acidosis, and stroke-like episodes (MELAS), Leigh Syndrome, Krabbe Disease, Canavan Disease, Metachromatic Leukodystrophy (MLD), Adrenoleukodystrophy (ALS), Nieman-Pick Discase, Alexander Disease, Pelizacus-Merzbacher Disease (PMD), Zellweger Syndrome, Parkinsonism-type diseases, Motor neuron diseases comprising one or more of amyotrophic lateral sclerosis (ALS) and progressive supranuclear palsy (PSP), multiple system atrophy, spinocerebellar ataxis and prion diseases. In an embodiment, the down-regulated pathways for treating neurodegenerative disease or disorder comprise one or more of TNF-alpha Signaling, TGF-beta Signaling, Complement, WNT Beta Catenin, Hypoxia, IFN Gamma Response, Inflammatory response, IL-6 JAK Stat3 signaling, Unfold Protein Response, P53 pathway, Apoptosis pathway, Oxidative Phosphorylation, ROS pathway, KRAS signaling Up, Fatty Acid Metabolism, Hemc metabolism, MTORC1 Signaling and Peroxisomc.

In an embodiment, the inflammatory pain or disorder comprises one or more of pain secondary to any tissue injury caused by any etiology, rheumatoid arthritis, lower back pain, osteoarthritis pain, gout pain and neuropathic pain. In an embodiment, the down-regulated pathways for treating the inflammatory pain or disorder comprise one or more of IL-6 JAK Stat3 signaling, TNF-alpha Signaling, TGF-beta Signaling, IFN Gamma Response, Inflammatory response, oxidative phosphorylation and ROS pathway.

In an embodiment, the steatohepatitis comprises one or more of Non-alcoholic steatohepatitis (NASH) and Alcohol-associated steatohepatitis. In an embodiment, the down-regulated pathways for treating the steatohepatitis comprise one or more of TNF-alpha signaling via NFKB, ROS pathway, TGF-beta signaling, inflammatory response, IL-2 STAT5 signaling, IL-6 JAK/STAT3 signaling, oxidative phosphorylation, MTORC1 signaling, hypoxia, fatty acid metabolism, apoptosis, interferon gamma and xenobiotic metabolism

In an embodiment of the present disclosure, methods to up-regulate interferon alpha response pathway for stimulating an antiviral and anti-cancer response comprising administering a pharmaceutical composition to a subject are provided. In an embodiment, the pharmaceutical composition comprises a stem cell mobilizer, and an immunosuppressive agent or non-immunosuppressive FK binding protein ligand. In an embodiment, the immunosuppressive agent or non-immunosuppressive FK binding protein ligand is administered at a sub-immunosuppressive dose. In an embodiment, the stem cell mobilizer comprises AMD 3100. In an embodiment, the dose range of the stem cell mobilizer, or AMD3100, is about 0.12 mg/kg to about 0.48 mg/kg. In an embodiment, the dose of the stem cell mobilizer, or AMD3100, is about 0.14 mg/kg, about 0.16 mg/kg, about 0.18 mg/kg, about 0.20 mg/kg, about 0.22 mg/kg, about 0.24 mg/kg, about 0.26 mg/kg, about 0.28 mg/kg, about 0.30 mg/kg, about 0.32 mg/kg, about 0.34 mg/kg, about 0.36 mg/kg, about 0.38 mg/kg, about 0.40 mg/kg, about 0.42 mg/kg, about 0.44 mg/kg, about 0.46 mg/kg, or about 0.48 mg/kg. In an embodiment, the stem cell mobilizer, or AMD3100, plasma concentration range at about 1, about 3, and/or about 8 hours is about 200 ng/mL to about 1,000 ng/mL. In one embodiment, the stem cell mobilizer, or AMD3100, plasma concentration at about 1, about 3, and/or about 8 hours is about 200 ng/mL, about 250 ng/ml, about 300 ng/ml, about 350 ng/ml, about 400 ng/mL, about 450 ng/mL, about 500 ng/ml, about 550 ng/ml, about 600 ng/ml, about 650 ng/ml, about 700 ng/mL, about 750 ng/ml, about 800 ng/ml, about 850 ng/mL, about 900 ng/mL, about 950 ng/mL or about 1,000 ng/mL.

In an embodiment, the immunosuppressive agent or non-immunosuppressive FK binding protein ligand comprises Tacrolimus. In an embodiment, the dose range of the immunosuppressive agent or non-immunosuppressive FK binding protein ligand, or Tacrolimus, is about 0.0025 mg/kg to about 0.01 mg/kg. In an embodiment, the dose of the immunosuppressive agent or non-immunosuppressive FK binding protein ligand, or Tacrolimus, is about 0.0030 mg/kg, about 0.0035 mg/kg, about 0.0040 mg/kg, about 0.0045 mg/kg, about 0.0050 mg/kg, about 0.0055 mg/kg, about 0.0060 mg/kg, about 0.0065 mg/kg, about 0.0070 mg/kg, about 0.0075 mg/kg, about 0.0080 mg/kg, about 0.0085 mg/kg, about 0.0090 mg/kg, about 0.0095 mg/kg, or about 0.01 mg/kg.

In an embodiment, the immunosuppressive agent or non-immunosuppressive FK binding protein ligand, or Tacrolimus, plasma concentration range at about 1, about 3, about 8, about 12 and/or about 24 hours is about 1 ng/ml to about 7 ng/ml. In an embodiment, the immunosuppressive agent or non-immunosuppressive FK binding protein ligand, or Tacrolimus, plasma concentration range at about 1, about 3, about 8, about 12 and/or about 24 hours is about 1 ng/mL, about 2 ng/ml, about 3 ng/ml, about 4 ng/ml, about 5 ng/ml, about 6 ng/ml or about 7 ng/mL.

In an embodiment, the virus and/or cancer comprise one or more of various viruses' infection diseases comprising one or more of hepatitis B and C, virus infection related cancers, lymphoma, malignant melanoma (skin cancer), genital warts, hairy cell leukemia (blood cell cancer), and Kaposi sarcoma (AIDS-related tumor). In an embodiment, the up-regulated pathway for stimulating the antiviral and anti-cancer response comprises interferon alpha response.

In an embodiment of the present disclosure, methods to up-regulate heme metabolism pathway for promoting heme synthesis in a subject comprising administering a pharmaceutical composition to a subject are provided. In an embodiment, the pharmaceutical composition comprises a stem cell mobilizer, and an immunosuppressive agent or non-immunosuppressive FK binding protein ligand. In an embodiment, the immunosuppressive agent or non-immunosuppressive FK binding protein ligand is administered at a sub-immunosuppressive dose. In an embodiment, the stem cell mobilizer comprises AMD 3100. In an embodiment, the dose range of the stem cell mobilizer, or AMD3100, is about 0.12 mg/kg to about 0.48 mg/kg. In an embodiment, the dose of the stem cell mobilizer, or AMD3100, is about 0.14 mg/kg, about 0.16 mg/kg, about 0.18 mg/kg, about 0.20 mg/kg, about 0.22 mg/kg, about 0.24 mg/kg, about 0.26 mg/kg, about 0.28 mg/kg, about 0.30 mg/kg, about 0.32 mg/kg, about 0.34 mg/kg, about 0.36 mg/kg, about 0.38 mg/kg, about 0.40 mg/kg, about 0.42 mg/kg, about 0.44 mg/kg, about 0.46 mg/kg, or about 0.48 mg/kg. In an embodiment, the stem cell mobilizer, or AMD3100, plasma concentration range at about 1, about 3, and/or about 8 hours is about 200 ng/ml to about 1,000 ng/mL. In one embodiment, the stem cell mobilizer, or AMD3100, plasma concentration at about 1, about 3, and/or about 8 hours is about 200 ng/ml, about 250 ng/ml, about 300 ng/mL, about 350 ng/mL, about 400 ng/mL, about 450 ng/ml, about 500 ng/ml, about 550 ng/ml, about 600 ng/ml, about 650 ng/ml, about 700 ng/ml, about 750 ng/mL, about 800 ng/mL, about 850 ng/mL, about 900 ng/mL, about 950 ng/ml or about 1,000 ng/mL.

In an embodiment, the immunosuppressive agent or non-immunosuppressive FK binding protein ligand comprises Tacrolimus. In an embodiment, the dose range of the immunosuppressive agent or non-immunosuppressive FK binding protein ligand, or Tacrolimus, is about 0.0025 mg/kg to about 0.01 mg/kg. In an embodiment, the dose of the immunosuppressive agent or non-immunosuppressive FK binding protein ligand, or Tacrolimus, is about 0.0030 mg/kg, about 0.0035 mg/kg, about 0.0040 mg/kg, about 0.0045 mg/kg, about 0.0050 mg/kg, about 0.0055 mg/kg, about 0.0060 mg/kg, about 0.0065 mg/kg, about 0.0070 mg/kg, about 0.0075 mg/kg, about 0.0080 mg/kg, about 0.0085 mg/kg, about 0.0090 mg/kg, about 0.0095 mg/kg, or about 0.01 mg/kg.

In an embodiment, the immunosuppressive agent or non-immunosuppressive FK binding protein ligand, or Tacrolimus, plasma concentration range at about 1, about 3, about 8, about 12 and/or about 24 hours is about 1 ng/ml to about 7 ng/mL. In an embodiment, the immunosuppressive agent or non-immunosuppressive FK binding protein ligand, or Tacrolimus, plasma concentration range at about 1, about 3, about 8, about 12 and/or about 24 hours is about 1 ng/ml, about 2 ng/mL, about 3 ng/ml, about 4 ng/mL, about 5 ng/ml, about 6 ng/ml or about 7 ng/ml.

In an embodiment, the promotion of heme synthesis comprises a deficiency in an enzyme or substrate that leads to an accumulation or increase of intermediates of heme synthesis in blood, tissues, and/or urine for treatment of a group of disorders comprising porphyria. In an embodiment, the up-regulated pathway for promoting heme synthesis comprises heme metabolism.

Without further elaboration, it is believed that one skilled in the art, using the preceding description, can utilize the present disclosure to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated and are intended to be purely illustrative and are not intended to limit the scope of what the inventors regard as their disclosure. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process.

### Example 1

The composition used in the study is a combination product of Plerixafor (AMD3100) and Tacrolimus (FK506) with excipients for subcutaneous injection (referred to herein as “Composition A” or “MRG-001”).

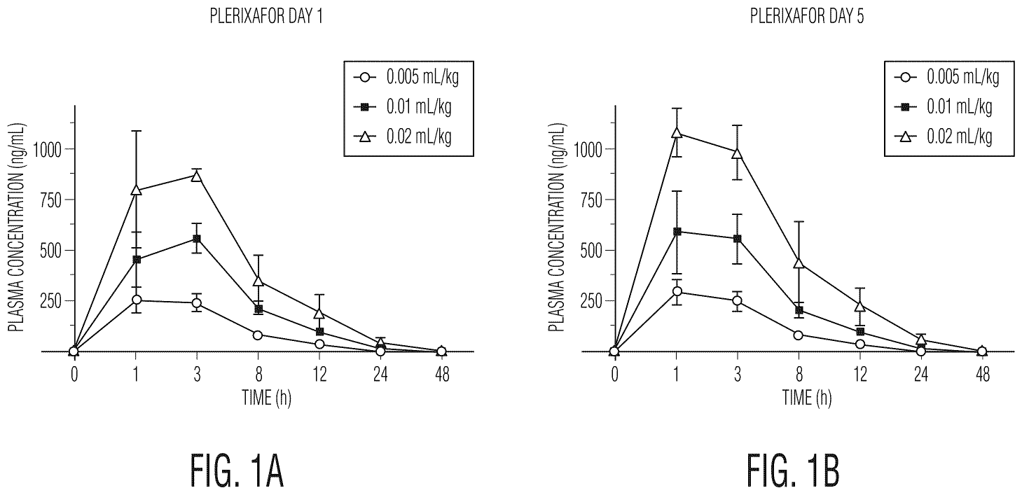

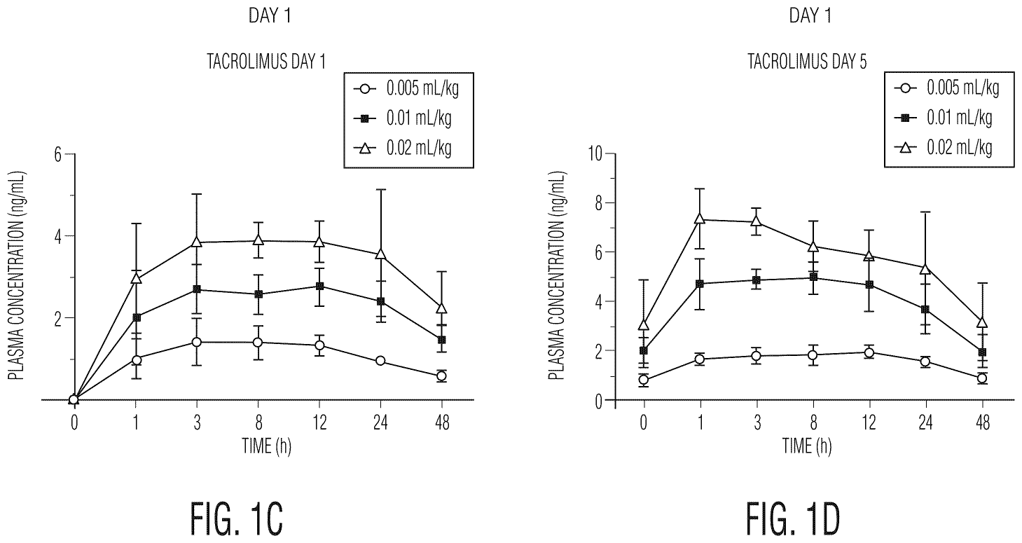

### Detailed Description of FIGS. 1-7:

FIG. 1: Composition A pharmacokinetics. A: Time profiles for plerixafor following SC administration of a single dose of 0.005, 0.01, and 0.02 mL/kg Composition A on Day 1.B: Time profiles for plerixafor following QAD SC administration of 0.005, 0.01, and 0.02 mL/kg Composition A on day 5. C: Time profiles for tacrolimus following SC administration of a single dose of 0.005, 0.01, and 0.02 mL/kg Composition A on Day 1. D: Time profiles for tacrolimus following QAD SC administration for 5 days of 0.005, 0.01, and 0.02 mL/kg Composition A on Day 5. n=4 per group.

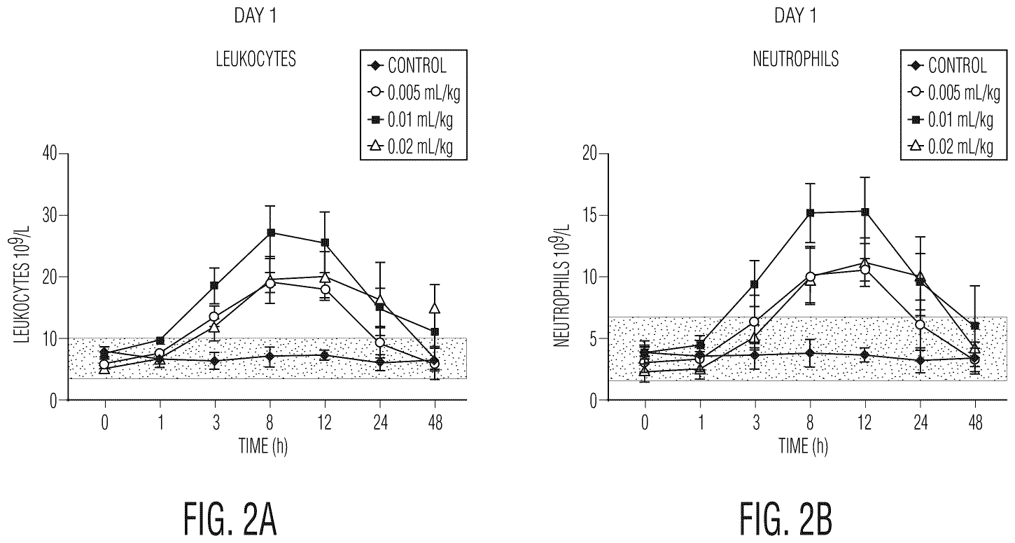

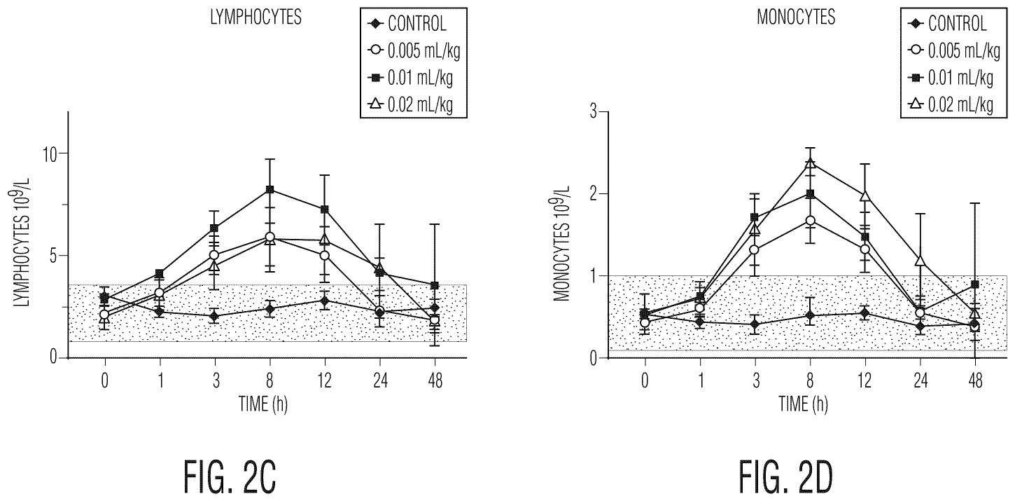

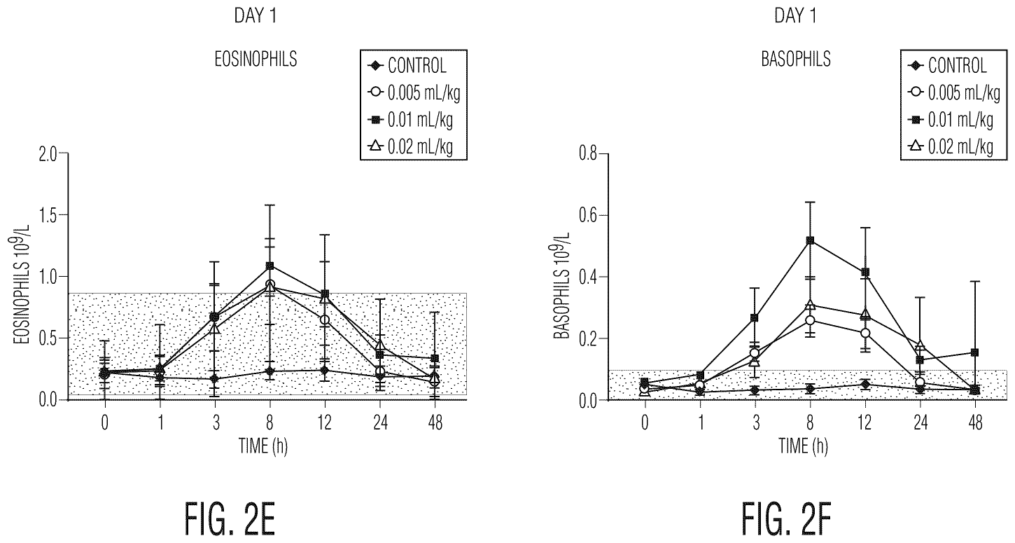

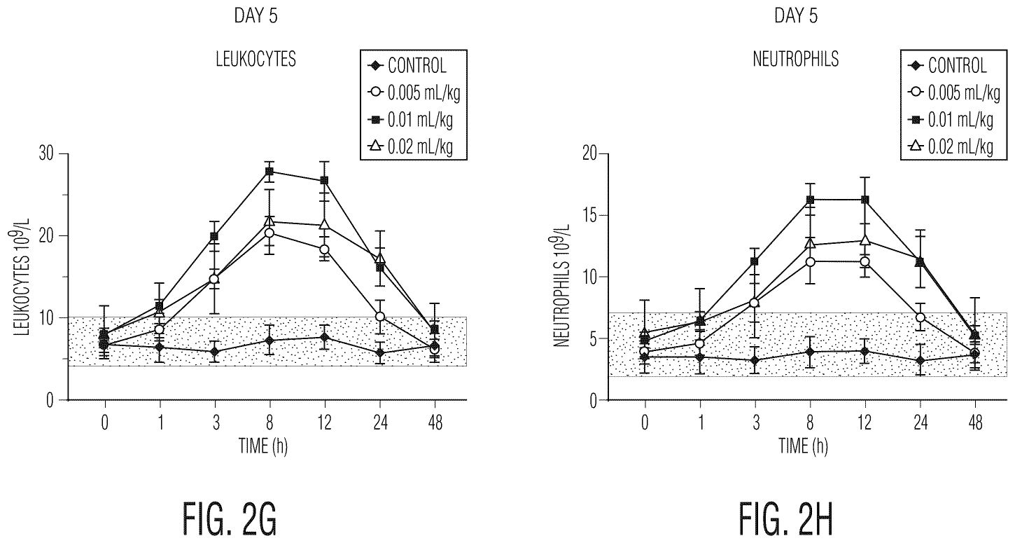

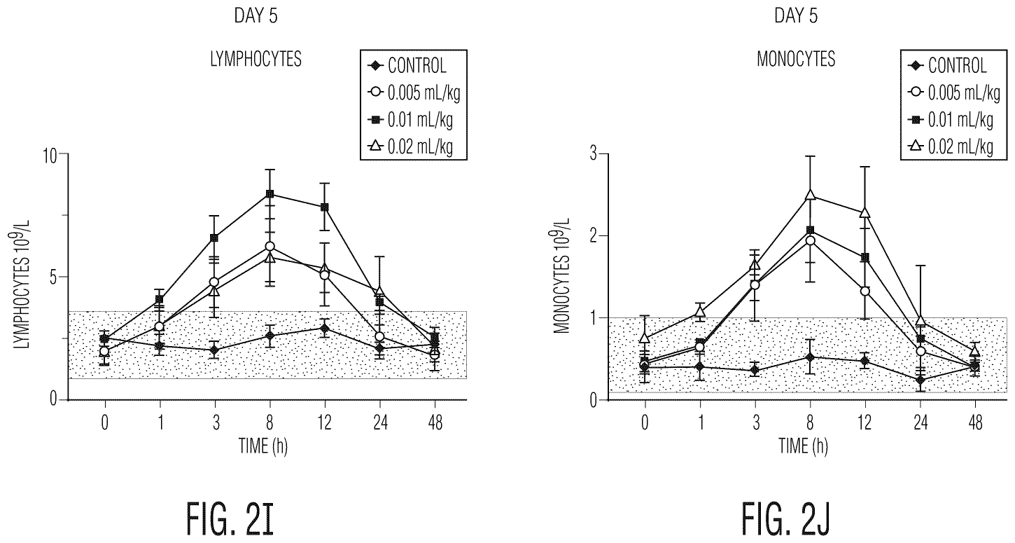

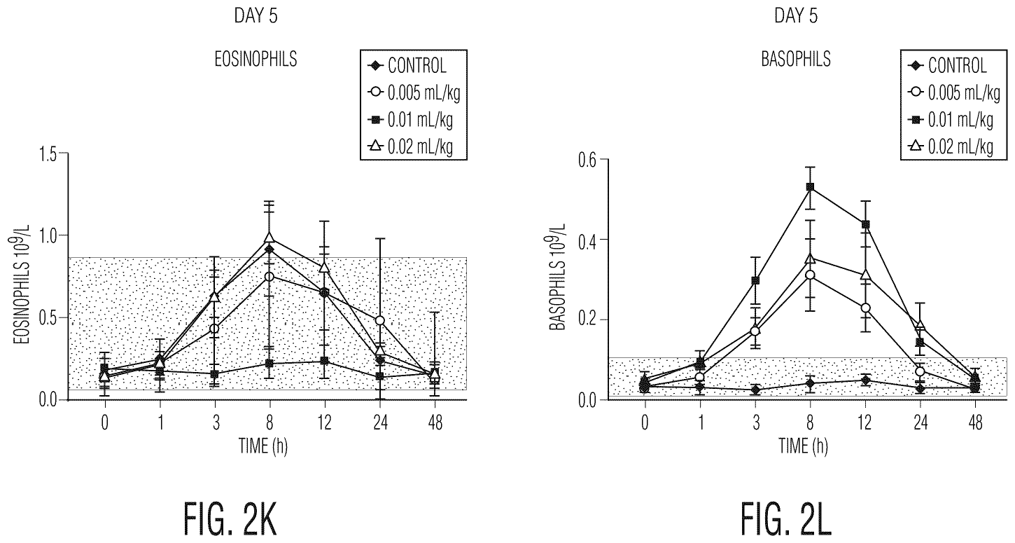

FIG. 2: White blood cell mobilization with Composition A in healthy subjects. Subjects received QAD SC injection of saline (placebo control, n=6), low-dose (0.005 mL/kg), mid-dose (0.01 mL/kg) or high-dose (0.02 mL/kg) Composition A (n=4/group). Venous blood was collected to determine the white blood cell differential count at several time intervals. A-F: white blood cells on day 1 following a single dose SC injection of saline or Composition A. G-L: white blood cells on day 5 following the third dose SC injection of saline or Composition A. Each value represents the mean±SD. The grey area represents the normal reference range.

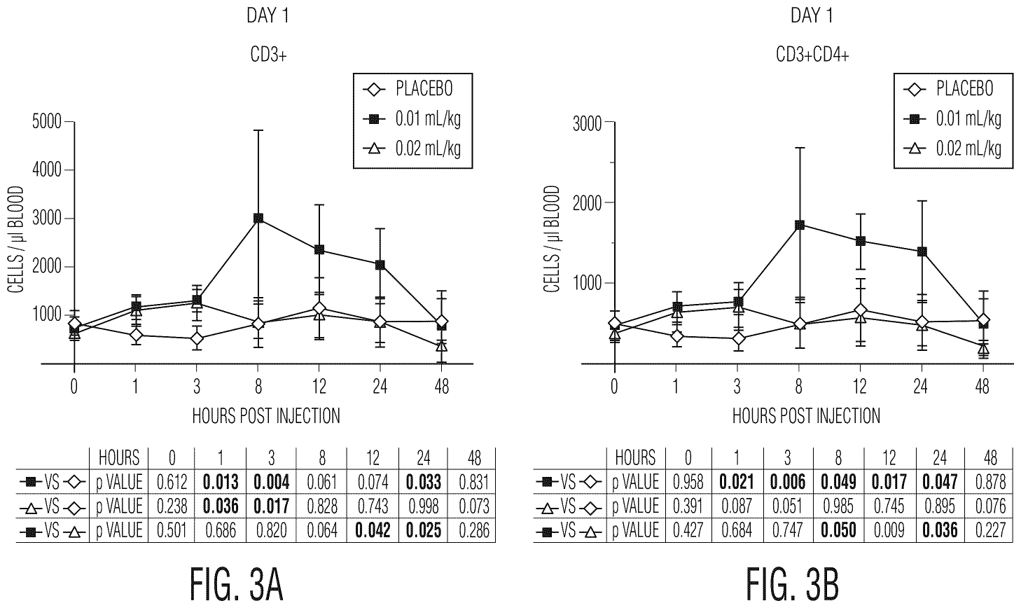

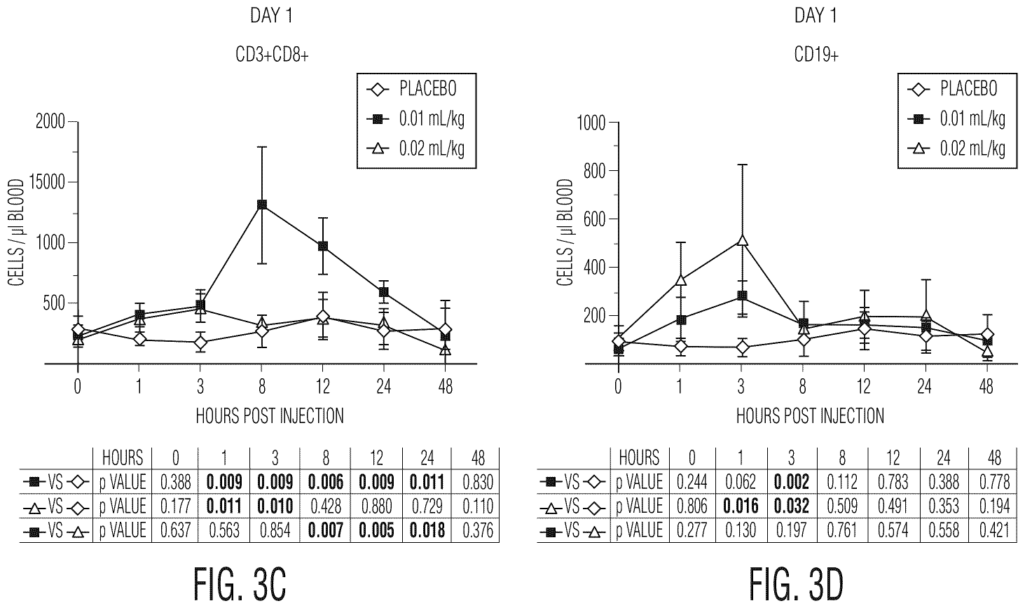

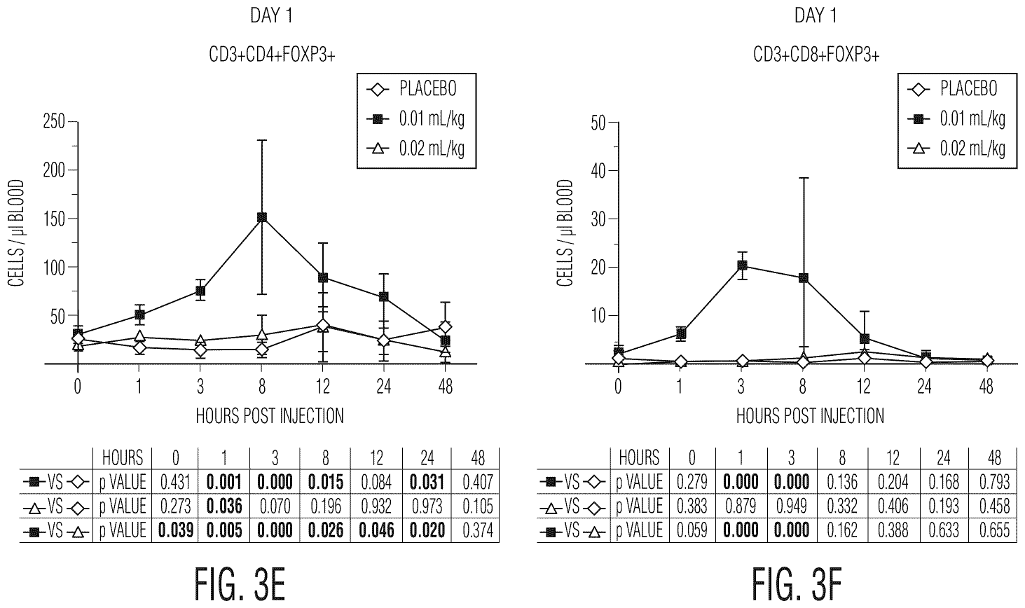

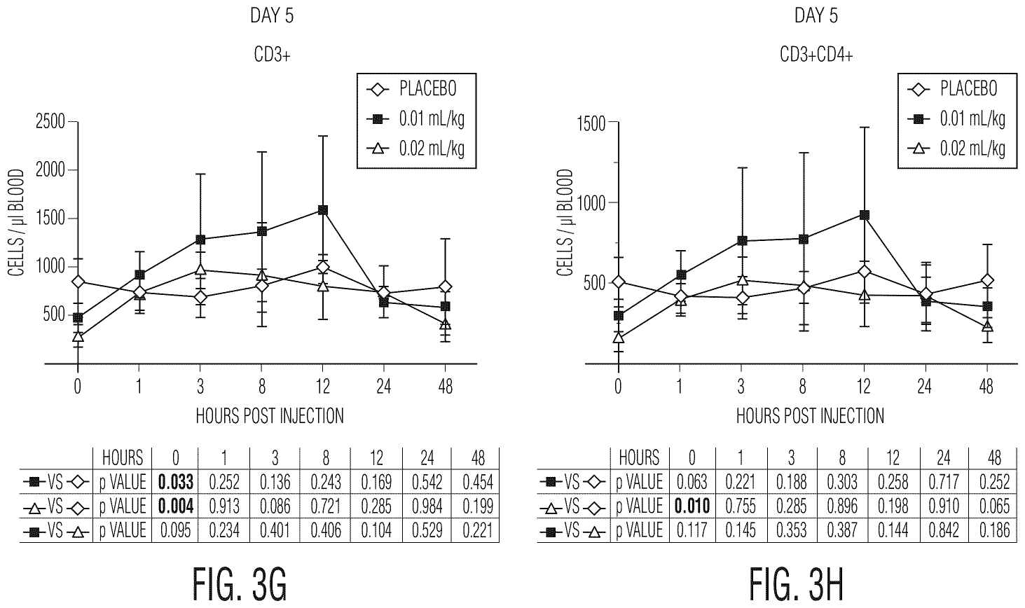

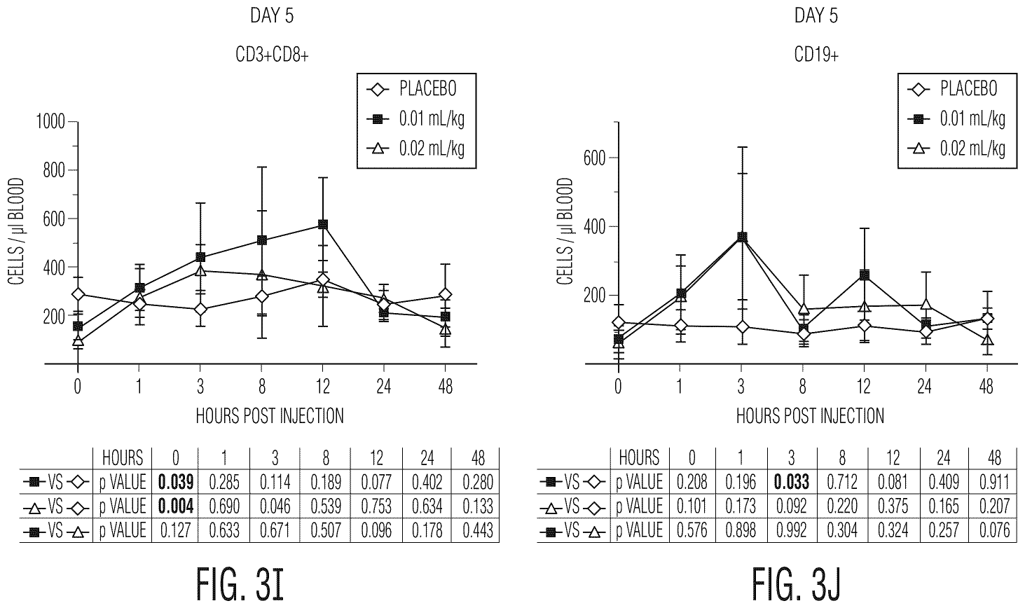

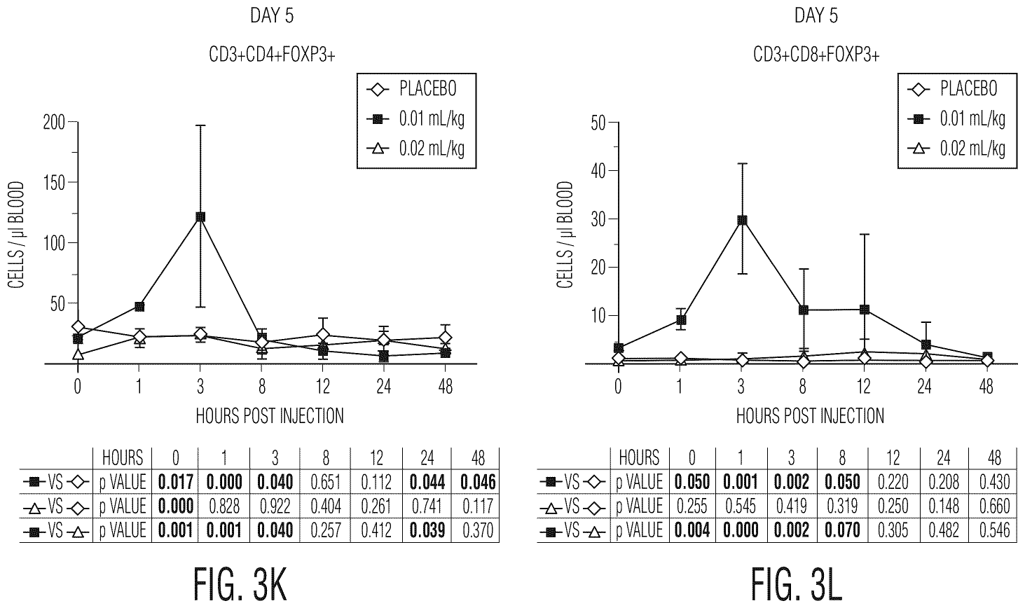

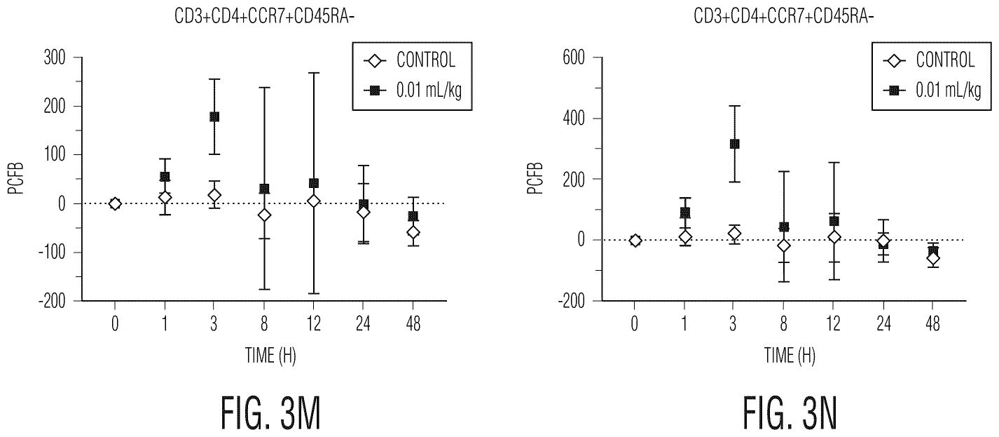

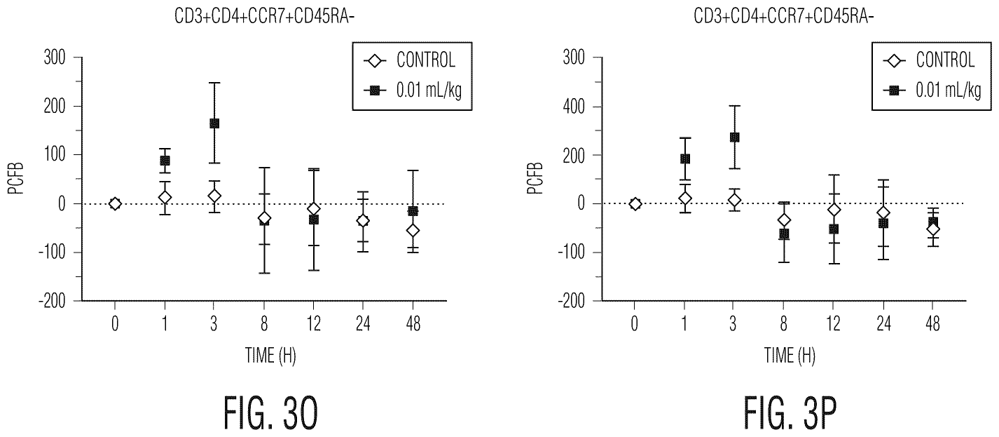

FIG. 3: Lymphocyte mobilization with Composition A in healthy subjects. Subjects received QAD SC injection of saline (placebo control), mid-dose (0.01 mL/kg) or high-dose (0.02 mL/kg) Composition A (n=4/group). Venous blood was collected and PBMCs were isolated at different time intervals on day 1 following a single dose SC injection of saline or Composition A and on day 5 following the third dose. Lymphocyte populations (CD3+, CD3+CD4+, CD3+CD8+, CD19+, CD3+CD4+Foxp3+ and CD3+CD8+Foxp3+) in PBMC were analyzed by flow cytometry. At each blood draw timepoint, absolute number of circulating lymphocyte subsets, such as CD3+ T cells, was calculated by multiplication of CD3+ cell fraction of PBMC by the total circulating PBMC count. A-F: Lymphocyte populations in peripheral blood on day 1 following a single dose SC injection of saline or Composition A. G-L: Lymphocyte populations in peripheral blood on day 5 following the third dose. Each value represents the mean±SD. M-P: percent change from baseline (PCFB) over time (hours) of CD3+CD4+CCR7+CD45RA⁻ population with 0.01 mL/kg of Composition A.

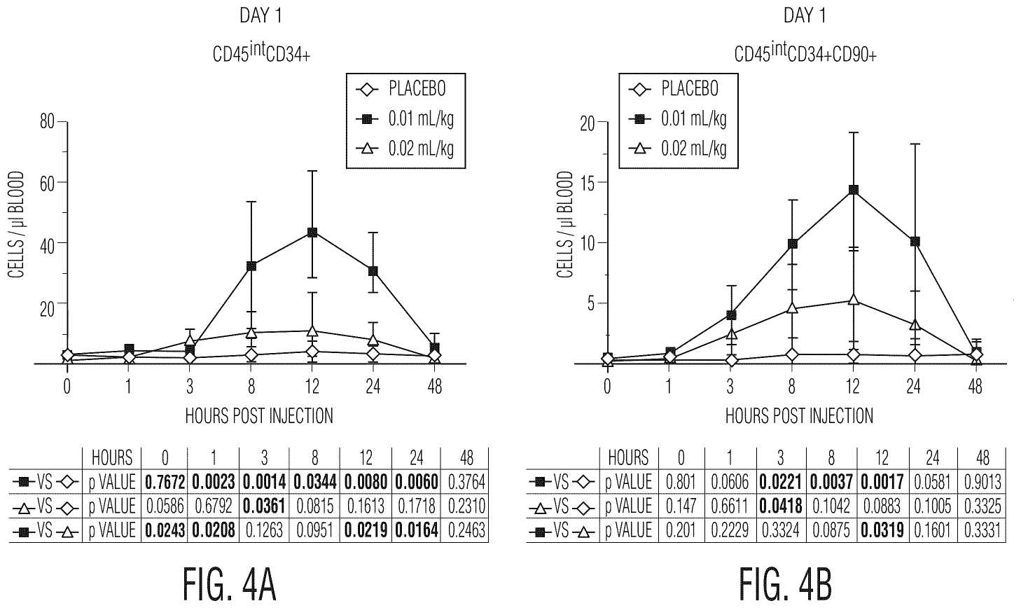

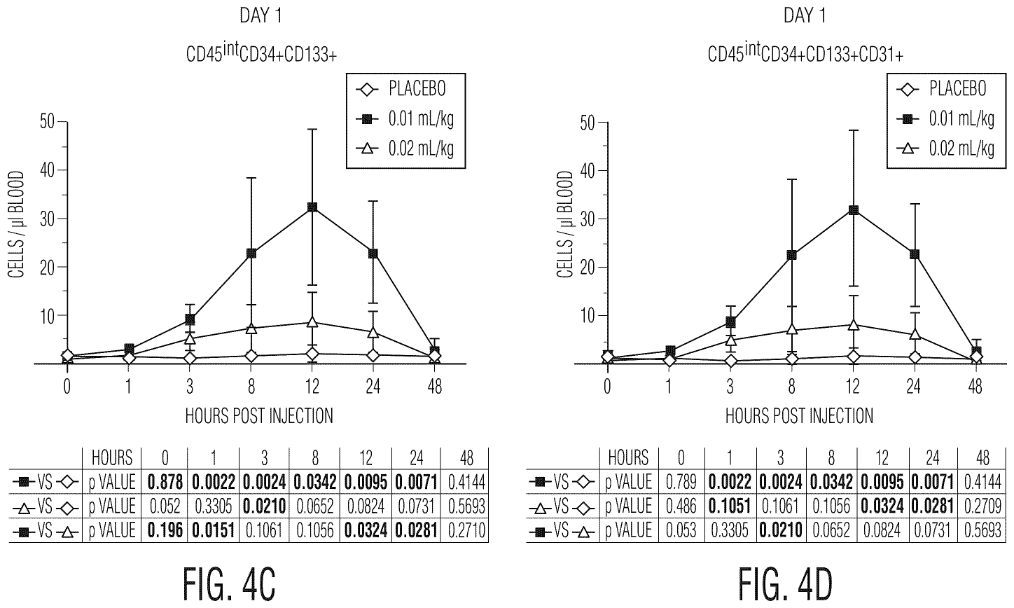

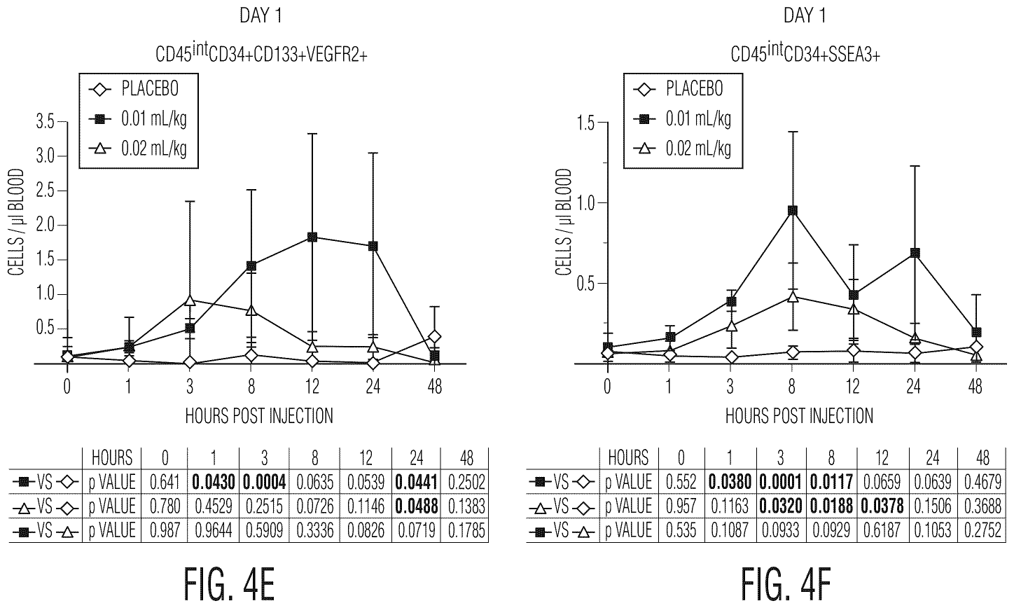

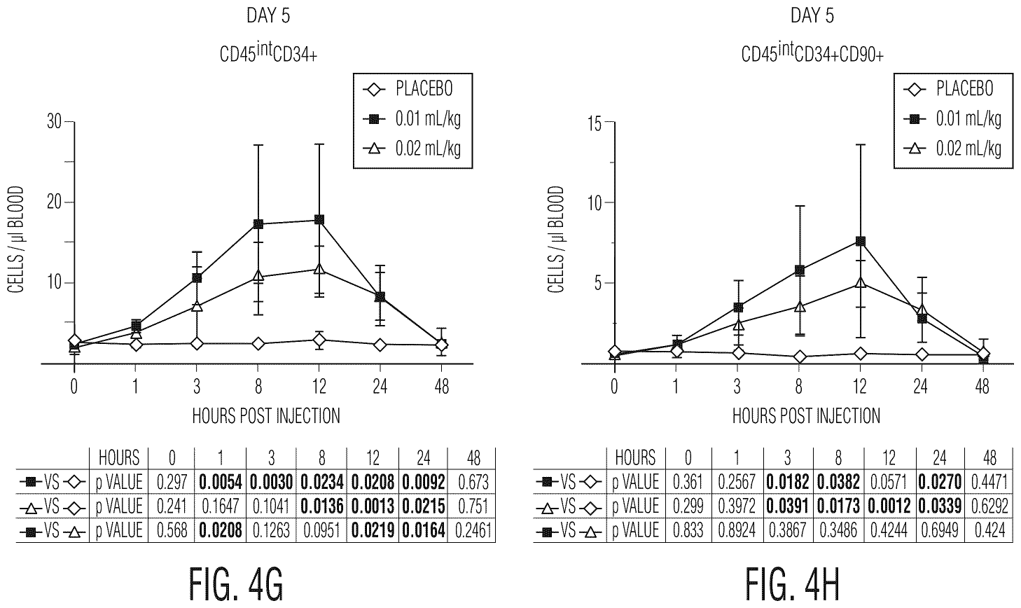

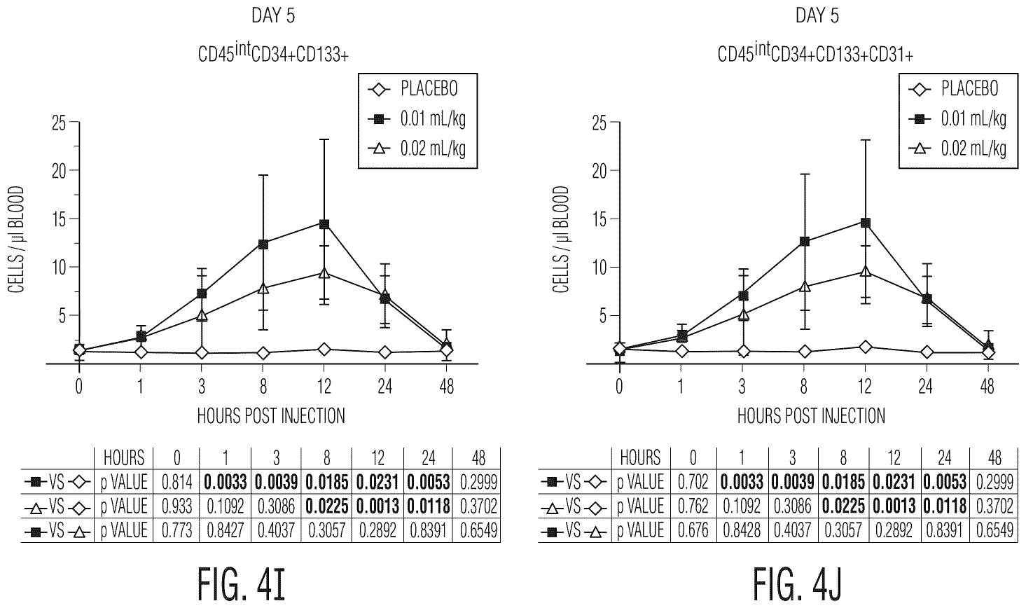

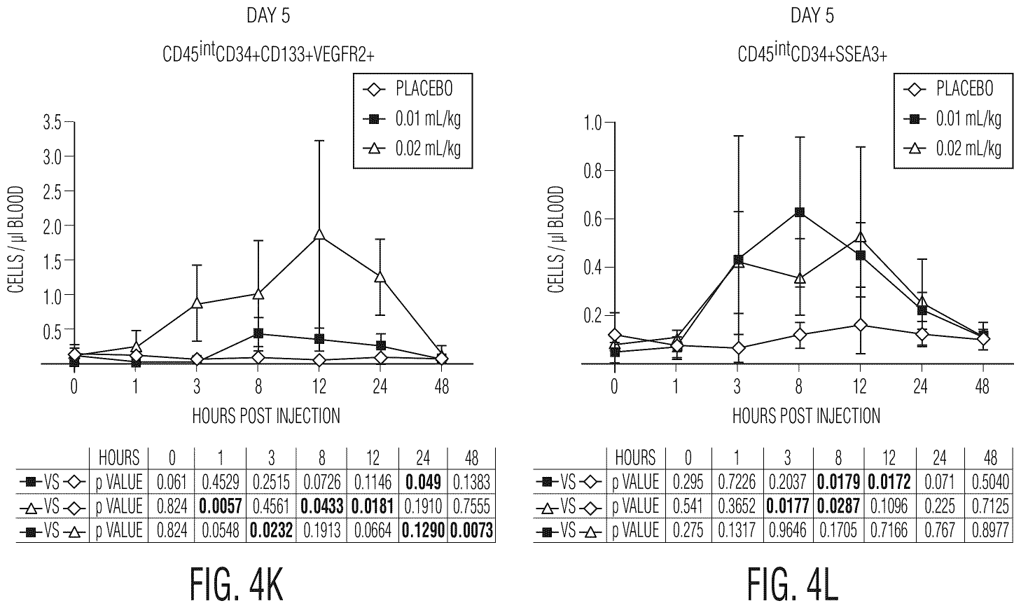

FIG. 4: Subjects received QAD SC injection of saline (placebo control), mid-dose (0.01 mL/kg) or high-dose (0.02 mL/kg) Composition A (n=4/group). Venous blood was collected and PBMCs were isolated at different time intervals on day 1 following a single dose SC injection of saline or Composition A and on day 5 following the third dose. Stem cell populations (CD45^{Int}CD34+, CD45^{Int}CD34+CD90+, CD45^{Int}CD34+CD133+, CD45^{Int}CD34+CD133+CD31+, CD45ItCD34+CD133+VEGFR2+, CD45^{Int}CD34+SSEA3+) in PBMC were analyzed by flow cytometry. At each blood draw timepoint, absolute number of circulating stem cells, such as CD45^{Int}CD34+HSCs, was calculated by multiplication of CD45^{Int}CD34+ cell fraction of PBMC by the total circulating PBMC count. A-F: Stem cells in peripheral blood on day 1 following a single dose SC injection of saline or Composition A. G-L: Stem cells in peripheral blood on day 5 following the third dose. Each value represents the mean±SD.

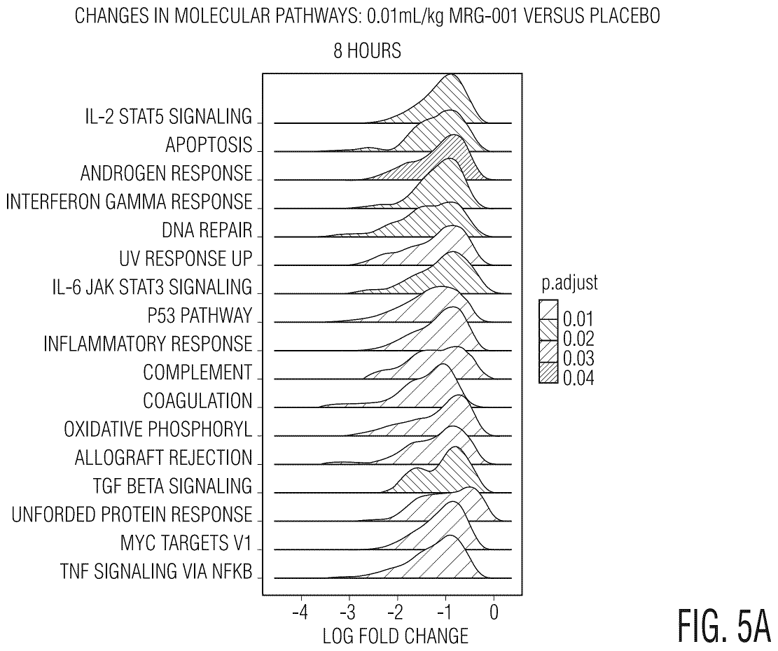

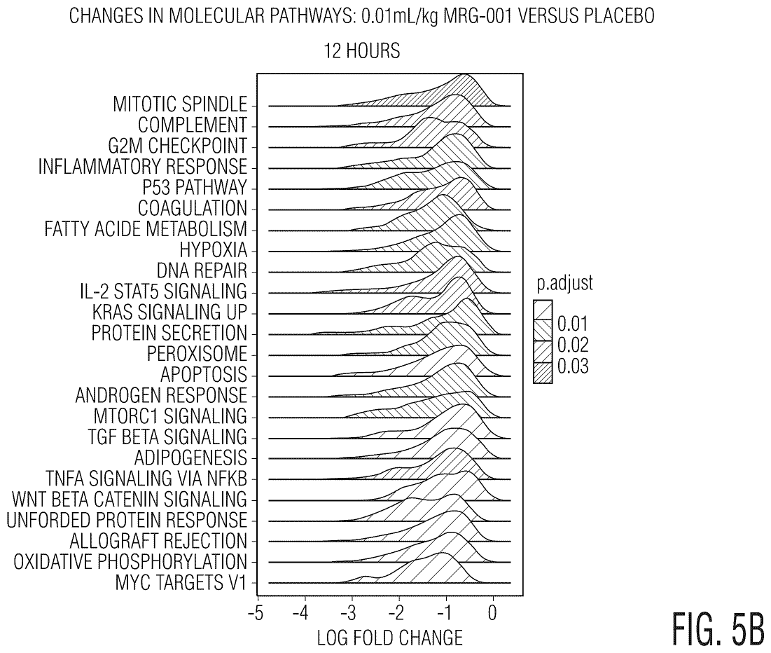

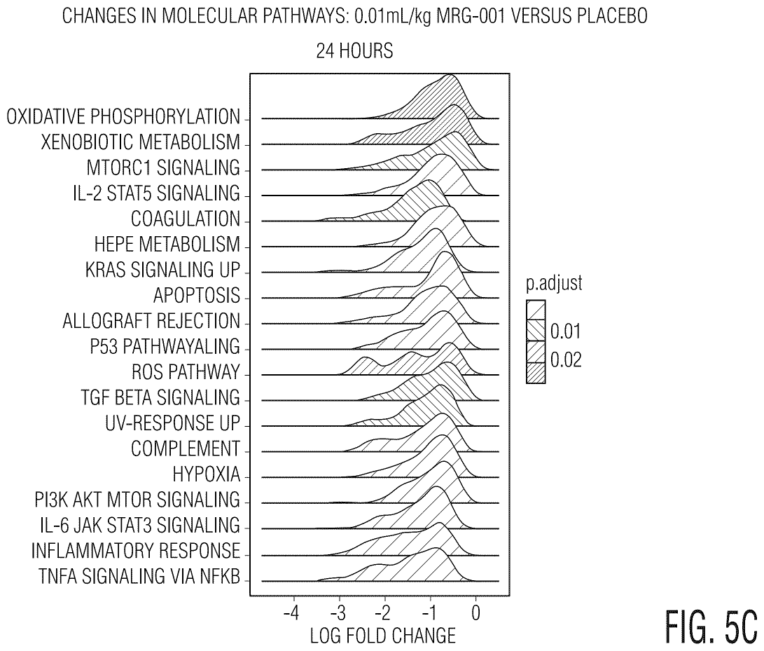

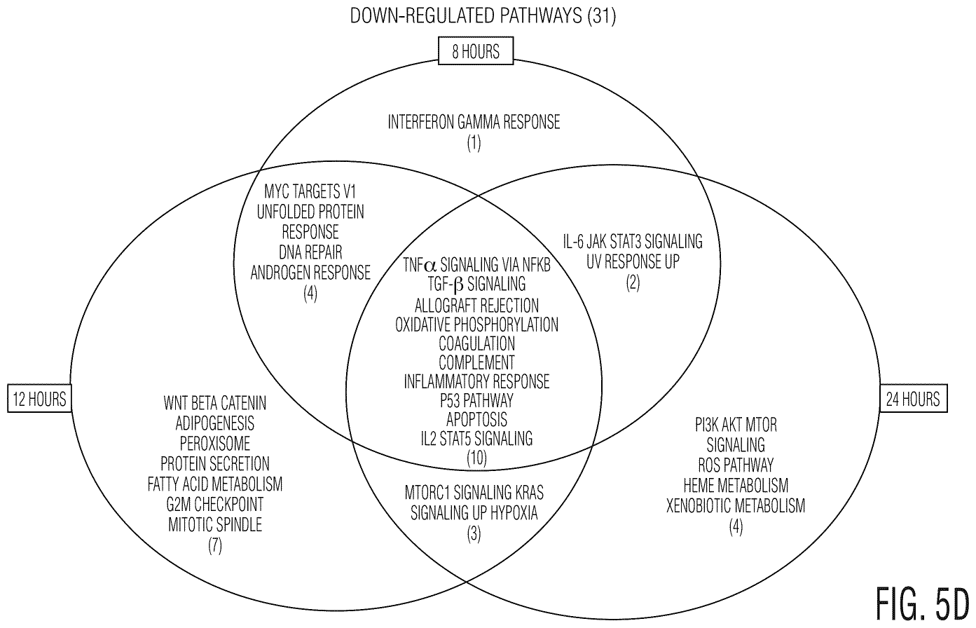

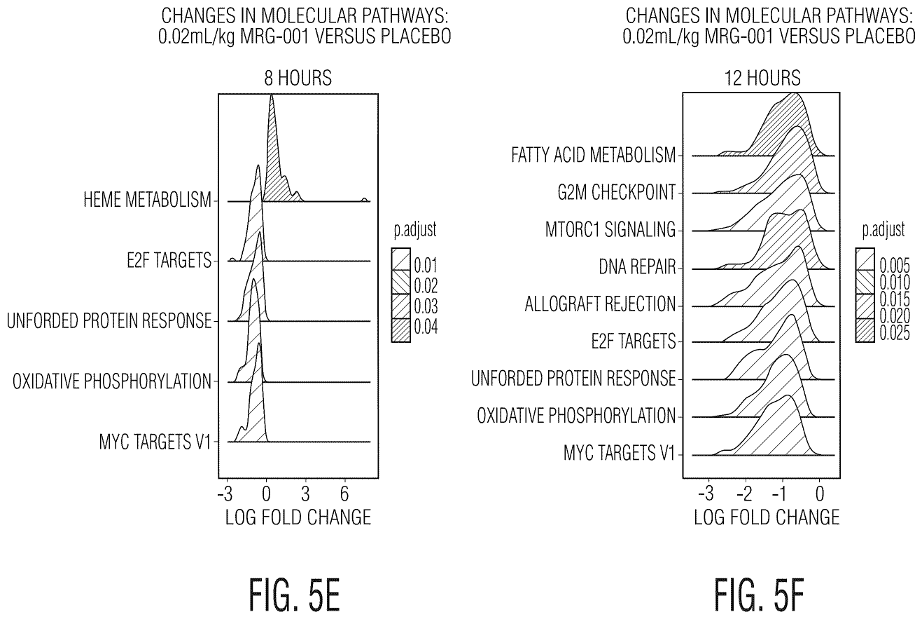

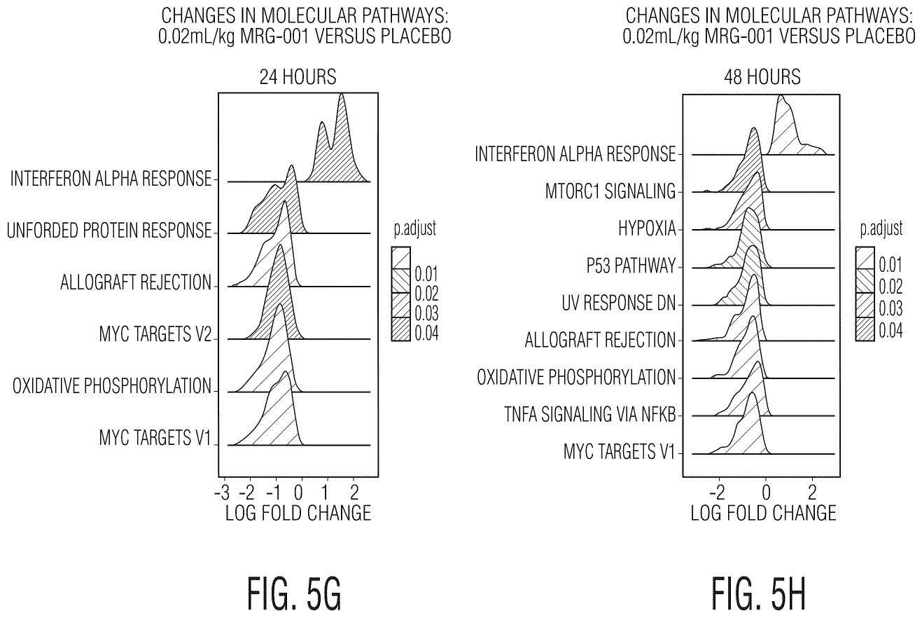

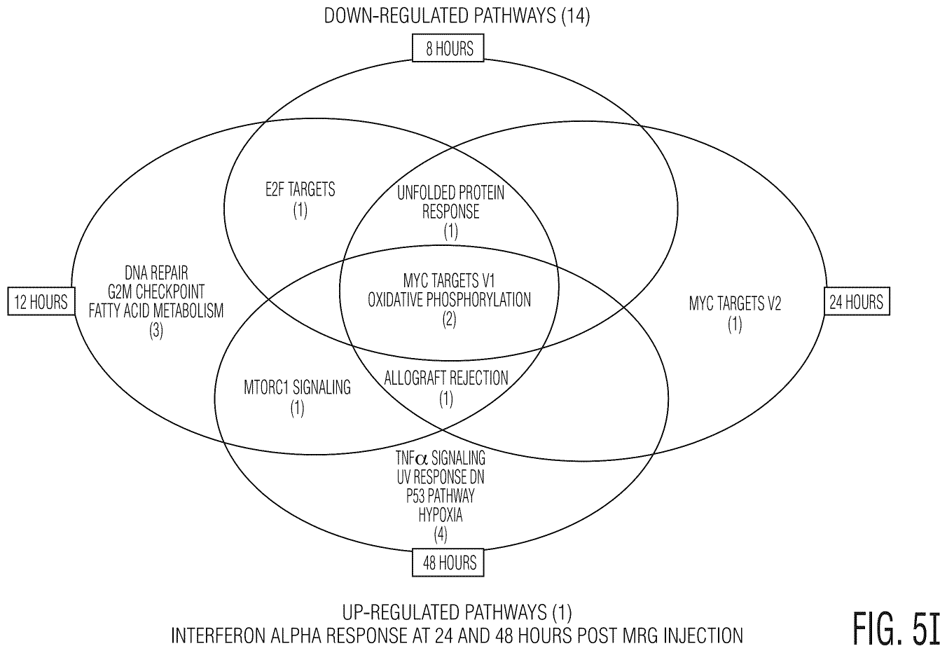

FIG. 5: Regulated molecular pathways by Composition A. RNA sequencing transcriptome studies of peripheral blood mononuclear cells (PBMC) were performed at different time points following a single dose SC injection of saline (placebo) or Composition A. A-D: Gene set enrichment analysis for interpreting gene expression profiles revealed 17, 24 and 19 down-regulated pathways at 8, 12 and 24 hours following administration of 0.01 mL/kg Composition A. A total of 31 down-regulated pathways were recognized and summarized in a Venn diagram. E-I: Gene set enrichment analysis showed 4, 9, 5 and 8 down-regulated pathways at 8, 12, 24 and 48 hours, and 1 upregulated pathway at 8, 24 and 48 hours following administration of 0.02 mL/kg Composition A. Total 14 down-regulated and 2 up-regulated pathways were recognized, and down-regulated pathways were summarized in a Venn diagram.

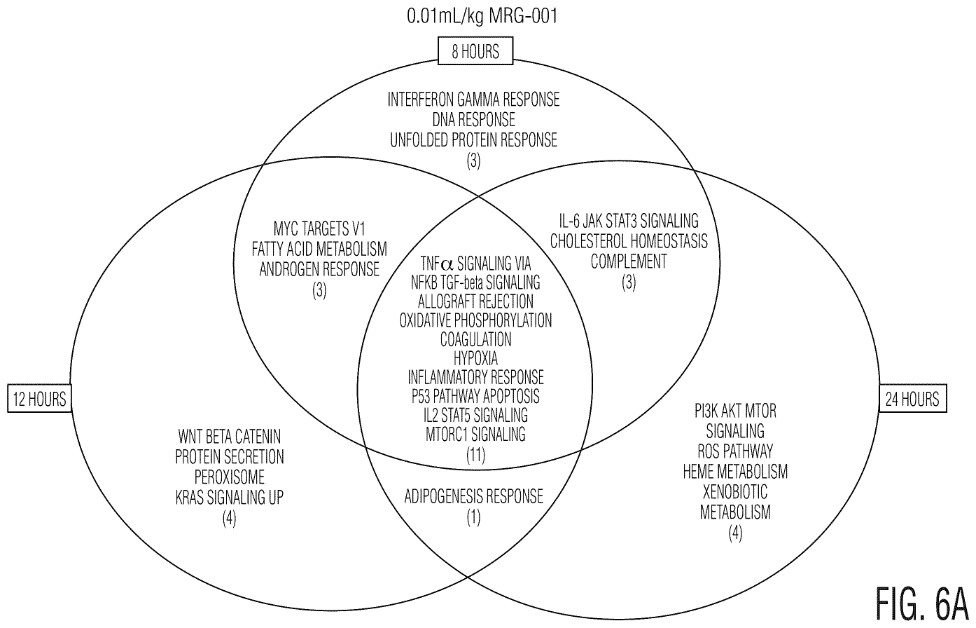

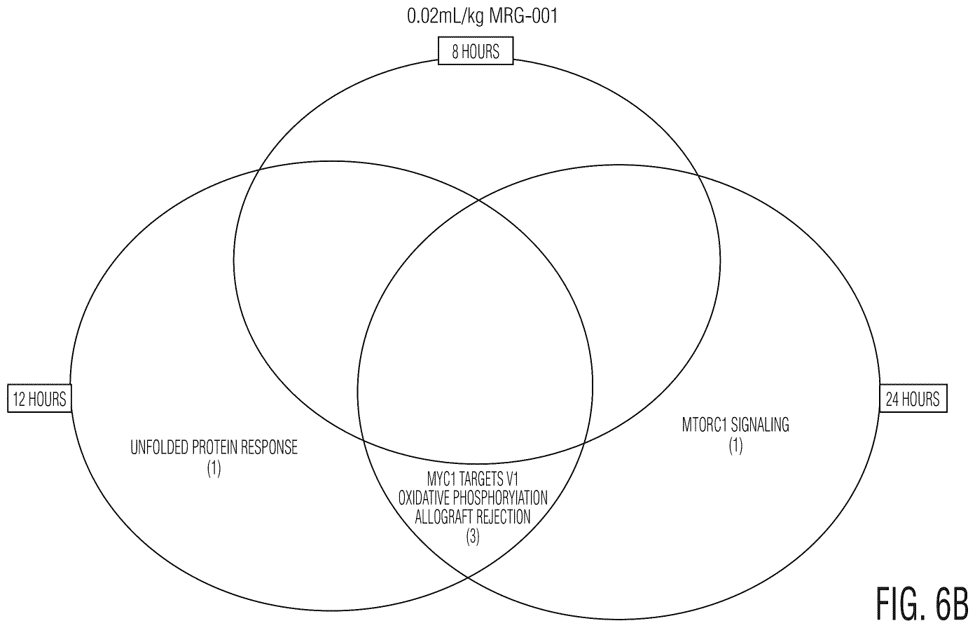

FIG. 6: Venn diagram analysis of molecular pathways down-regulated by Composition A at 8, 12 and 24 hours post injection compared to pre-dose. (A) 0.01 mL/kg Composition A. Compared to pre-dose, down-regulated pathways at 8 hours, 12 hours and 24 hours are 20, 19 and 19, respectively. Total 29 pathways are down-regulated. The shared down-regulated pathways at different time points were analyzed using the Venn diagram analysis. As illustrated in each panel, the common down-regulated pathways at different time points are 11. (B) 0.02 mL/kg Composition A. Compared to pre-dose, down-regulated pathways at 8, 12 and 24 hours are 0, 4 and 4, respectively. Total 5 pathways are down regulated, and shared down-regulated pathways at 12 and 24 hours are 3.

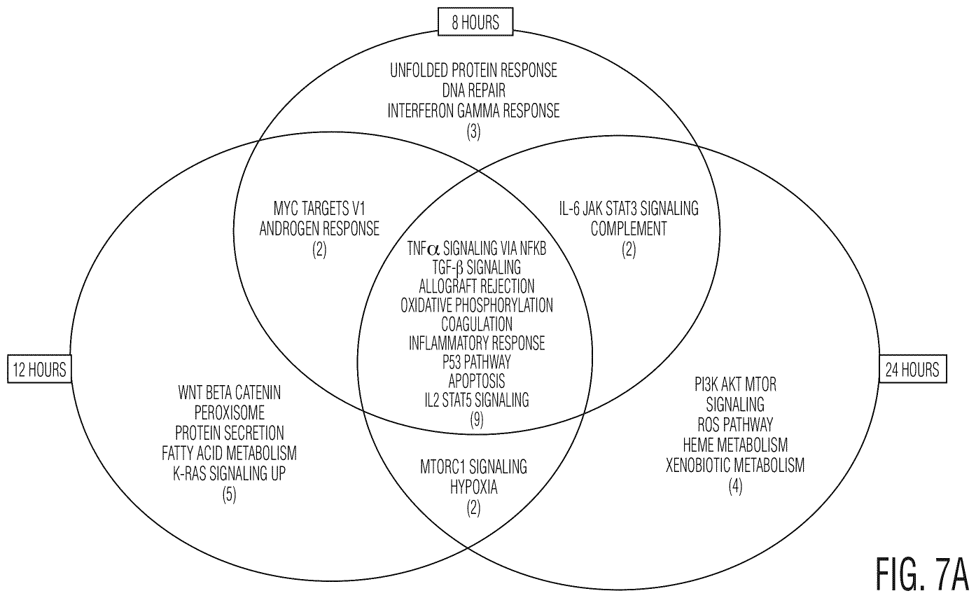

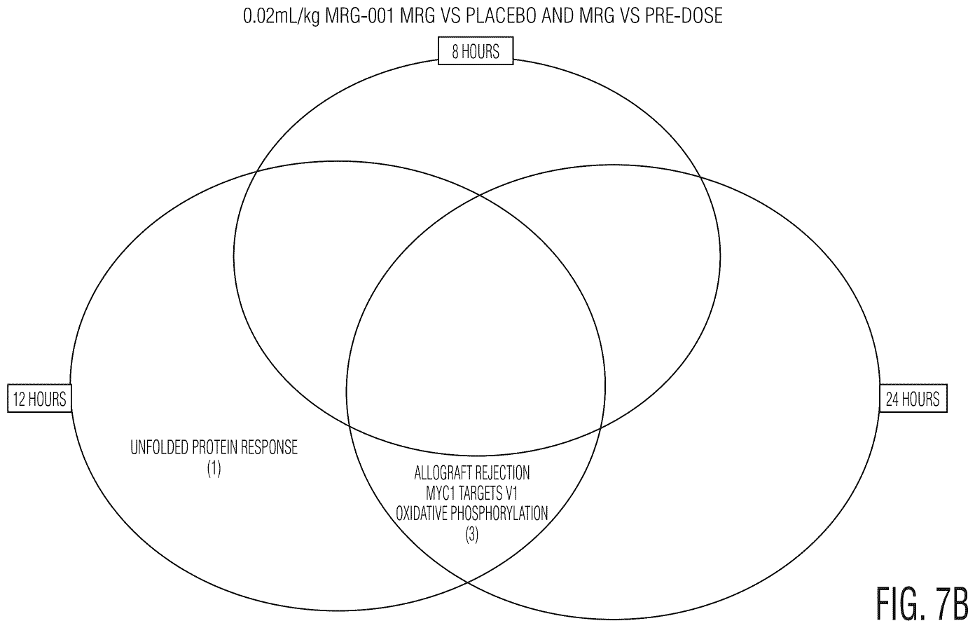

FIG. 7: Venn diagram analysis of common down-regulated pathways between Composition A vs Placebo and Composition A vs Pre-dose. (A) 0.01 mL/kg Composition A. Common down-regulated pathways between 0.01 mL/kg Composition A versus Placebo and 0.01 mL/kg Composition A versus Pre-dose at 8, 12 and 24 hours are 16, 18 and 17, respectively. Total common down-regulated pathways are 27. As illustrated in each panel, the shared common down-regulated pathways at different time points are 9. (B) 0.02 mL/kg Composition A. Common down-regulated pathways between 0.02 mL/kg Composition A versus Placebo and 0.02 mL/kg Composition A versus Pre-dose at 8, 12 and 24 hours are 0, 4 and 3, respectively. Total common down-regulated pathways are 4. As illustrated in each panel, the shared common down-regulated pathways at 12 and 24 hours are 3.

### Study Design

This first-in-human, single-center, randomized, double-blind, placebo-controlled, multiple-ascending dose (MAD), phase I study (NCT04646603) in healthy volunteers was performed at the San Antonio Clinical Research Unit, ICON plc, in San Antonio, Texas, in the United States of America between February 2021 and May 2021.

The study was conducted in accordance with Good Clinical Practice as defined by the International Conference on Harmonization and in line with the ethical principles of the Declaration of Helsinki, European Union Directive 2001/20/EC and the US Code of Federal Regulations, Title 21, Part 50. The clinical protocol, amendments, and informed consent forms were approved by the US FDA and by an independent Ethics Committee, IntegReview IRB (Austin, TX, USA), before study initiation and throughout the study. All participants provided written informed consent and could withdraw from the study at will.

### Study Population

Male and female participants between 18-45 years with a body mass index of 18.8-32 kg/m2, and healthy with no clinically significant abnormalities as determined by medical history, physical examination, 12-lead electrocardiogram (ECG), and clinical laboratory evaluations, were eligible for inclusion. All participants were required to have a negative SARS-COV-2 test by real-time polymerase chain reaction within the previous 96 hours before admission.

Non-pregnant, non-lactating females of childbearing potential who agreed to use medically acceptable forms of birth control from the screening visit until the end of the study visit were eligible. Females were required to have a negative serum pregnancy test before admission. Male participants were eligible if they agreed to use effective contraception methods from the signing of the ICF until at least 8 weeks after the last dose of the study drug.

Exclusion criteria were related to a medical history of cardiovascular, respiratory, hepatic, renal, gastrointestinal, endocrine, neurological, immunological, psychiatric disorder(s) and laboratory findings of HIV, hepatitis B, or C. Subjects were excluded if they received concomitant medication that could affect the pharmacokinetics of the investigational product (IP). Other exclusion criteria included subjects unwilling to avoid the use of alcohol within 48 hours before screening and for the duration of the study and to abstain from nicotine use from screening until the end of the study. The complete list of inclusion and exclusion criteria is listed in the supplementary text S1. A total of 18 subjects were planned to be included in the study.

### Investigational Treatments and Dose Regimen

The composition used in the study is a combination product of Plerixafor (AMD3100) and Tacrolimus (FK506) with excipients for subcutaneous injection (referred to herein as “Composition A” or “MRG-001”). Each 1 ml vial of Composition A contains 24 mg/ml Plerixafor and 0.5 mg/mL Tacrolimus. Sterile 0.9% sodium chloride solution for subcutaneous injection served as placebo.

The dose selection was based on established clinical experience with each active pharmaceutical ingredient (API), effective dosages in animal models as well as its safety profile in nonclinical toxicological studies in two species. Subjects were enrolled in 3 sequential cohorts (cohort 1:0.005 mL/kg, cohort 2:0.01 mL/kg, cohort 3:0.02 mL/kg Composition A or 0.9% saline placebo) of 6 subjects each, of which 4 subjects were randomized to the Composition A group and 2 subjects to the placebo group. Subjects received subcutaneous injections in the abdominal area on days 1, 3 and 5 and were required to fast 1-hour pre-dose and 1-hour post-dose. Follow-up visits were conducted on days 6 and 7 and subjects were discharged and returned on day 12 for the end-of-study visit.

Each cohort included a sentinel dosing group of 2 subjects (1 Composition A and 1 placebo) dosed at the same time, with dosing of the remaining cohort following review of 24 hours safety results of both sentinel subjects. Dose escalation to the next cohort occurred after review of all available safety, tolerability and pharmacokinetic data by the data safety review committee consisting of the principal investigator, medical monitor, pharmacokinetics expert and the study sponsor's physician.

### Safety Assessment

Safety assessments included adverse events (AE) monitoring, vital signs monitoring (systolic and diastolic blood pressure, pulse rate, oral body temperature and respiratory rate), physical examination, concomitant medication assessment, 12-lead electrocardiography and laboratory evaluations (clinical chemistry, hematology, coagulation and urinalysis). The injection site was assessed for erythema, pain, swelling and numerous other parameters according to a standardized local injection site reaction score every 1, 6, 12 and 24 hours after injection. The safety of the subjects was assessed until discharge and during the end-of-study visit on day 12.

### Pharmacokinetic Assessment

Plerixafor. Blood samples were obtained from all subjects at the following time points: before dosing, at 1, 3, 8, 12, 24 and 48 hours after injection. Blood samples were also obtained daily until discharge on day 7. Plasma from each sample was isolated and stored in −80° C. condition until shipment. The analysis of plasma for the quantitation of plerixafor using a validated method by liquid chromatography with tandem mass spectrometry (LC-MS/MS) was performed by NorthEast Biolab (Hamden, CT, USA) in compliance with principles of Good Laboratory Practice (GLP) Standards as outlined the US FDA Title 21 CFR Part 58; Good Clinical Practice (US FDA & ICH GCP Guidance) Standards, the Declaration of Helsinki, and the US FDA Guidance for COVID-19 Studies. The data were acquired using Analyst® v1.7 software and the following pharmacokinetic parameters were calculated: peak plasma concentration (Cₘₐₓ), time to peak plasma concentration (Tₘₐₓ), terminal half-life (t_{1/2}), trough concentration (C_{trough}) and area under the plasma concentration-time curve over dosing interval (AUCₜₐᵤ).

Tacrolimus. Blood samples were obtained from all subjects in parallel as for plerixafor. Whole blood from each sample was stored in −80° C. condition until shipment. The analysis of K2EDTA whole blood for the quantitation of tacrolimus using a validated method by LC-MS-MS was performed by Worldwide Clinical Trials, Bioanalytical Services (Austin, TX, USA) in compliance with principles of GLP standards as referenced above.

Study data were collected and similar pharmacokinetic parameters as above were calculated using Analyst® (Version 1.4.2 or 1.6.1, Applied Biosystems/MDS Sciex) and evaluated with Watson Laboratory Information Management System™ (LIMS; Version 7.2.0.03, Thermo Fisher Scientific) software.

### Pharmacodynamics Assessment

Blood samples were obtained from certain subjects at pre-dose and at 1, 3, 8, 12, 24 and 48 hours for flow cytometry. A stem cell panel and an immune cell panel were developed for this study. Briefly, whole blood was lysed and then washed with PBS. Cells were then stained with a viability dye, washed, and blocked with human Fc block. Surface staining was performed with an extracellular antibody cocktail. For the stem cell panel, after surface staining, the cells were washed and fixed. For the immune cell panel, after surface staining, cells were washed, fixed, and permeabilized. Permeabilized cells were stained with an intracellular antibody cocktail containing FoxP3 antibodies. After intracellular staining, cells were washed and then fixed with stabilizing fixative before acquisition. Counting beads were added to be able to count the absolute number of cells. A list of the antibodies can be found in the supplemental table S3. Data were acquired using a BD LSR Fortessa (BD, San Jose, CA) and analyzed with FlowJo version 10.7.1 and Graphad Prism version 9.0. At each blood draw timepoint, absolute circulating immune cell or stem cell count, such as CD45dimCD34+, was calculated by multiplication of CD45dimCD34+ cell fraction of PBMC ((lymphocyte and monocytes in complete blood count (CBC)) by the total circulating PBMC count. Unfortunately, multiple PBMC samples from the 0.005 mL/kg Composition A cohort were degraded and unusable for both FACS analysis and RNA sequencing due to logistical delays, thus we were unable to include a low-dose cohort for immune and stem cell analysis.

### RNA Purification & Next Generation Sequencing

To understand the molecular mechanisms of Composition A, next-generation sequencing on RNA isolated from PBMCs was performed and gene expression changes were quantified. Sequencing occurred at multiple time points after injection to define the dynamics of gene expression changes between the different dose groups. Gene expression changes were pooled per timepoint per dose Composition A group and compared to the pooled pre-dose gene expression of that specific dose group and to the placebo-treated group.

RNA Extraction. Samples (snap-frozen white blood cell pellets) were received on dry ice and stored at −80° C. until processing commenced. Samples were randomized before extraction. In total, samples from 15 subjects (5 from the placebo group, 2 from 0.005 mL/kg Composition A, 4 from 0.01 mL/kg Composition A and 4 from the 0.02 mL/kg Composition A dose group) were sequenced. The Lexogen 008 split RNA extraction kit was used to extract RNA. Samples were characterized by UV-Vis spectrophotometry (Nanodrop2000c, Thermo Fisher), the RNA integrity was assessed on a Fragment Analyzer System using the DNF-471 RNA Kit (15nt) (Agilent).

Library Preparation. Sequencing-ready libraries were produced after randomization using a QuantSeq 3′ mRNA-Seq Library Prep Kit FWD for Illumina (015UG009V0260) with Globin Block Module following procedures for degraded RNA, as outlined by the manufacturer's instructions (Lexogen QuantSeQ_Illumina). Indexed library preparation was performed to allow for multiplexed sequencing. For library preparation, 100 ng of extracted RNA samples were used as an input. Prepared libraries were quality controlled and quantified on a Fragment Analyzer using HS NGS Fragment Kit (1-6000 bp). A sequencing-ready pool of indexed libraries was prepared according to these quantifications.

Sequencing. Sequencing was performed on an Illumina NextSeq2000 with a 100cyc P2 flow cell sequencing kit at Lexogen GmbH.

Sequencing quality control and adapter trimming. Using cutadapt version 1.18, the reads of the sequencing run were scanned for adapter contaminations, continuous polyA sequences and continuous polyG sequences at the 3′ end and had the contaminations removed if they were found. The reads of the samples prior to adapter trimming and after adapter trimming were analyzed with FastQC version v0.11.7.

Alignment and read quantification. The reads were aligned to the spike-in complemented Ensembl release 94 of the Homo sapiens assembly GRCh38 from the Genome Reference Consortium. The alignment was performed with the splice-aware aligner STAR version 2.6.la. The alignments were quantified based on the annotations of Ensembl GRCh38.94 and the spike-in-specific annotations of Lexogen with the featureCounts software program version 1.6.4 of the subread analysis package.

### Differential Gene Expression Analysis

A differential gene expression analysis was conducted using DESeq2 (version v1.18.1). The analysis used the counts of unique alignments. Significance was determined at adjusted P<0.1.

Functional Enrichment Analysis. For gene set enrichment testing the hallmark data set of the molecular signature database has been used. The data used has been retrieved from the CRAN R package msigdbr 7.5.1. This is visualized in a ridgeline plot, where the density of these significantly enriched gene sets is plotted against their log-fold changes.

### Statistical Methods

Formal sample size calculations were not performed however with a total sample size of 18 healthy volunteers and a given incidence of a specific common AE of 1% in the general population the study would be able to detect an additional incidence of such AE caused by the use of the new drug of 11% with a power of 80%. Given the stage of development and the objective to determine the safety, PK and PD profiles of Composition A, 12 subjects were considered adequate to assess the safety and PK/PD profiles in this initial part of development.

All statistical tabulations and analyses were done using SAS Grid/SAS Linux: SAS® 9.4. Formal statistical tests were conducted at a 2-sided 5% significance level.

Disposition, demographics, baseline characteristics and all safety parameters were summarized by cohort and treatment. Placebo was pooled across cohorts for summary and analysis. The PK full set includes all subjects who received at least 1 dose of study treatment and have at least 1 plasma concentration data point. The PD set will include those subjects in the safety analysis set who have pre-dose and at least 1 of the post-dose PD parameter concentrations and have no events or deviations that would affect PD variables.

### Results

### Disposition of Subjects

A total of 21 subjects were randomized into the study and received at least 1 dose of their assigned study drug. A total of 18 subjects (85.7%) received all doses per protocol. Eighteen subjects (85.7%) completed the study while 3 subjects (14.3%) discontinued early. One subject (4.8%) withdrew consent and 2 subjects (9.5%) were discontinued due to protocol violations.

### Baseline Characteristics

The mean age of the subjects was 32.4 years (Table 1). Overall, females outnumbered males by 2:1. Each dose group also had more females than males enrolled. The majority of subjects were of Hispanic or Latino ethnicity (n=13, 61.9%), the remainder being Caucasian. The Composition A groups were all approximately half Hispanic or Latino while the pooled placebo was less diverse with 6 subjects (85.7%) of Hispanic or Latino ethnicity. The overall mean height was 166.27 cm. The dose groups were well-matched and within 5% of the overall mean.

The overall mean weight was 72.14 kg. The dose groups were all within 10% of the overall mean with the Composition A 0.005 mL/kg group being the lightest (69.23 kg) and the Composition A 0.01 mL/kg group being the heaviest (76.24 kg).

The overall BMI was 25.99 kg/m2. The dose groups were well-matched and within 5% of the overall mean.

### Safety and Treatment-Emergent Adverse Events

No deaths, no serious adverse events (SAE) or significant adverse events (AE) leading to discontinuation of the study or study drug were reported (Table 2). There were no clinically significant vital signs or ECG abnormalities. Overall 11 subjects (52.4%) reported 38 treatment-emergent adverse events (TEAE) after study drug treatment; 2 subjects (50.0%) reported 6 TEAE after treatment with Composition A 0.005 mL/kg, 3 subjects (60.0%) reported 5 TEAEs after treatment with Composition A 0.01 mL/kg, 5 subjects (100.0%) reported 26 TEAE after treatment with Composition A 0.02 mL/kg, and 1 subject (14.3%) reported 1 TEAE after treatment with placebo.

All TEAE were considered mild (n=34) or moderate (n=4) in severity; there were no serious TEAE. The majority of TEAE were definitely (9 subjects [42.9%], 15 TEAE), probably (2 subjects [9.5%], 2 TEAE), or possibly (7 subjects 33.3 [%], 16 TEAE) related.

In the Composition A 0.005 mL/kg group (n=4), 2 subjects (50.0%) reported 6 TEAE including diarrhea, dry mouth, injection site pain, and headache. In the Composition A 0.01 mL/kg group (n=5), 3 subjects (60.0%) reported 5 TEAE including diarrhea, nausea, injection site pain, and paresthesia. In the Composition A 0.02 mL/kg group (n=5), 5 subjects (100.0%) reported 26 TEAE including abdominal pain, diarrhea, nausea, influenza-like illness, injection site erythema, injection site hemorrhage, injection site pain, injection site swelling, vulvovaginal candidiasis, musculoskeletal chest pain, dizziness, headache, somnolence, and insomnia.

In the pooled placebo group (n=7), 1 subject (14.3%) reported 1 mild, definitely related TEAE of injection site pain. Injection site reaction TEAE were noted in all 3 Composition A groups, with the Composition A 0.02 mL/kg group having the greatest numbers. In the 0.005 mL/kg group, 1 subject had a mild TEAE of injection site pain that had a duration of 5 hours. Two subjects in the 0.01 ml/kg group each had 1 mild TEAE of injection site pain with a duration of 10 minutes and 2 hours 9 minutes. All 5 subjects in the 0.02 mL/kg group experienced injection site reaction TEAE. The most common injection site reaction TEAE was injection site pain (verbatim term: burning at the injection site) of mild (10 TEAE) or moderate (2 TEAE) intensity, with durations ranging from 15 minutes to 3 hours. The only other injection site-related TEAE were reported in a single subject receiving Composition A 0.02 mL/kg and included injection site erythema (mild), injection site swelling (mild), and injection site hemorrhage (moderate) all lasting approximately 1 day.

There were no clinically significant abnormalities for chemistry, cardiac troponins, coagulation or urinalysis parameters. The only significant abnormality, which had no clinical impact, was the increase of mean white blood cells (WBC) and its lineages in the peripheral circulation in subjects treated with Composition A, as was expected. On average the white blood cell counts in test subjects increased 2-4 folds from baseline, while the placebo subjects did not show any increase.

### Pharmacokinetics-Plerixafor and Tacrolimus

Plerixafor. The mean plasma concentration-time profiles of plerixafor increased with dose following a single SC dose of Composition A on Day 1 and following QAD for 5 days. The elimination appeared to be monophasic for all 3 doses following a single dose on Day 1 and following QAD dosing for 5 days. One treated subject had an aberrant value (BLQ) at 3 hours post-dose on Day 1 for plerixafor. As this sample was at the potential Tmax, PK parameters were excluded from the summaries and statistical analysis.

Plasma PK parameters for plerixafor following SC administration of a single dose of COMPOSITION A on Day 1 and QAD administration for 5 days are summarized in Table 3 and FIGS. 1A & B. The inter-subject variability as represented by the GeoCV % for Cmax and AUC was low (ranging from 7.5 to 23.1%) for all dose levels on Days 1 and 5.

Geometric mean peak plasma concentrations of plerixafor (Cmax) increased with increasing dose of COMPOSITION A on Day 1 and following QAD dosing for 5 days. The median time to Cmax was approximately 1 to 3 hours following cither a single dose on Day 1 and following QAD dosing for 5 days. The geometric mean tin ranged from 3 to 6 hours. Plerixafor was fully eliminated at 48 hours across all dosages. Plasma PK parameters for plerixafor following SC administration of a single dose of Composition A on Day 1 and QAD administration for 5 days are summarized in table 3.

Tacrolimus. Mean plasma tacrolimus concentration-time profiles following a single SC dose of Composition A on Day 1 and following QAD doses on Day 5 are presented in Table 4 and FIGS. 1C and D. The mean plasma concentration-time profiles of tacrolimus increased with dose following a single SC dose of Composition A on Day 1 and following QAD dosing for 5 days. The elimination phase was not present in many of the subjects during the 48-hour sample period for Day 1 and appeared to be monophasic following QAD dosing for 5 days.

Plasma PK parameters for tacrolimus following SC administration of a single dose of Composition A on Day 1 and following QAD dosing for 5 days are summarized in Table 4. The intersubject variability (GeoCV %) for Cₘₐₓ and AUC was low to moderate (ranging from 10.8 to 34.7%) for all dose levels on Days 1 and 5.

The GM peak plasma concentrations of tacrolimus (Cₘₐₓ) increased with increasing dose of Composition A on both Day 1 and following QAD dosing for 5 days. The median Tₘₐₓ was approximately 2 to 12 hours following either a single dose on Day 1 and following QAD dosing for 5 days. The GM tin ranged from 24 to 38 hours.

The C_{trough} concentrations of tacrolimus increased with an increasing dose of Composition A on both Day 1 and following QAD dosing for 5 days. The median C_{trough} was 0.58, 1.48, 2.24 ng/ml respectively with increasing dosages on Day 1. On Day 5 the C_{trough} concentrations were 0.90, 1.99 and 3.19 ng/mL.

In summary following SC administration of a single dose on Day 1 and following QAD dosing for 5 days of Composition A 0.005, 0.01, and 0.02 mL/kg (24 mg plerixafor plus 0.5 mg tacrolimus per mL), Cₘₐₓ and AUC for both plerixafor and tacrolimus increased in a dose-proportional manner. The tin for plerixafor was short for all doses (3 to 6 hours) and longer for tacrolimus (24 to 38 hours).

### Pharmacodynamics—Mobilization of Immune Cells and Bone Marrow Stem Cells

WBC mobilization. WBC count was increased by approximately 2 to 4 fold from baseline levels post-injection in all Composition A treated subjects, reached the peak levels between 8 to 12 hours and returned to baseline levels or near baseline levels at 48 hours (FIG. 2A). Interestingly, mid-dose Composition A (0.01 mL/kg) mobilized more leukocytes, neutrophils, lymphocytes and basophils at 3, 8 and 12 hours (peak levels) than high-dose (0.02 mL/kg) or low-dose (0.005 ml/kg) Composition A. Composition A consistently mobilized WBC in a similar pattern on day 5 after the third dose injection (FIG. 2B).

Mobilization of immunoregulatory T cells to peripheral blood. In the mid-dose group (0.01 mL/kg), the numbers of CD3+, CD4+, and CD8+ T cells in peripheral blood increased by approximately 2 to 3 fold from baseline with peak levels at 8 hours and declined back to placebo levels at 48 hours (FIG. 3A) while CD19+B cells increased 3 fold at 3 hours and declined back to baseline levels at 8 hours following first dose (day 1). Interestingly, CD3+CD4+Foxp3+ regulatory T cells increased 3, 5 and 10 fold at 1 hour, 3 hours and 8 hours and remained at higher levels (3 fold) at 24 hours following the first mid dose, while CD3+CD8+Foxp3+ regulatory T cells increased 10 and 30 fold at 1 hour and 3 hours and declined back to baseline levels at 24 hours. In the high dose group (0.02 mL/kg), the numbers of CD3+, CD4+, CD8+ T cells in peripheral blood increased by approximately 2 to 3 fold and CD19+B cells increased by 4 to 6 fold at 1 hour and 3 hours, and declined back to baseline levels at 8 hours following first high dose SC. However, neither CD4+Foxp3+ nor CD8+Foxp3+ regulatory T cells were increased in the high-dose group.

At day 5 (0 hours, prior to the third dose), the numbers of CD3+, CD4+, CD8+ T cells and CD4+Foxp3+ regulatory T cells were significantly lower in Composition A groups compared to the placebo group (FIG. 3B). The third dose of Composition A increased CD3+, CD4+ and CD8+ T cells to levels that were similar to the placebo group at 1 hour, but levels of these cells in blood were not significantly higher in Composition A treatment groups than the placebo group at different time points (p>0.05). There was no significant difference in CD19+B cells at day 5 (0 hours), and the mid-dose but not high-dose Composition A significantly increased CD19+B cells (3 fold) at 3 hours following the third dose. Consistently, CD3+CD4+Foxp3+ regulatory T cells increased 2 and 5 fold at 1 hour and 3 hours and declined to the placebo levels at 8 hours, while CD3+CD8+Foxp3+ regulatory T cells increased approximately 12, 112 and 80 fold at 1, 3 and 8 hours and declined to the placebo levels at 24 hours following third of mid-dose COMPOSITION A. High-dose COMPOSITION A did not increase Foxp3+ regulatory T cells.

The mid-dose of COMPOSITION A consistently increased CD4+Foxp3+ and CD8+Foxp3+ regulatory T cells on day 1 and day 5, suggesting its potential for immunoregulation.

Mobilization of bone marrow stem/progenitor cells to peripheral blood. In the mid-dose group (0.01 mL/kg), following administration of COMPOSITION A, CD45In CD34+ and CD45^{Int}CD34+CD133+ hematopoietic stem cells (HSCs) and CD45^{Int}CD34+CD133+CD31+ endothelial progenitor cells (EPCs) in peripheral blood increased at 1 hour, reached peak levels at 12 hours, remained at higher levels at 24 hours, and declined back to placebo levels at 48 hours (FIG. 4A). At 12 hours, these stem/progenitor cells increased approximately 15 to 17 fold from baseline levels (pre-dose) and the peak CD45^{Int}CD34+HSCs were 43.86/μL blood. CD45^{Int}CD34+CD90+ multipotent HSCs with long-term multilineage engraftment potential (15) increased approximately 7, 18 and 26-fold from baseline levels at 3, 8 and 12 hours. Compared to the placebo group, CD45^{Int}CD34+CD133+VEGFR2+ early endothelial progenitor cells increased approximately 3, 28 and 60 fold at 1, 3 and 24 hours, while stage-specific embryonic antigen 3 (SSEA3) expressing HSCs (CD45^{Int}CD34+SSEA3+) also increased 3, 8 and 12 fold at 1, 3 and 8 hours. In the high-dose group (0.02 mL/kg), stem cell populations were increased in a similar pattern but to a moderate degree, and the peak CD45^{Int}CD34+HSCs were 11/μL blood.

Following administration of the third dose at day 5, COMPOSITION A consistently mobilized stem/progenitor cells in a similar pattern (FIG. 4B), but the numbers of mobilized stem/progenitor cells were lower compared to day 1 and the peak CD45^{Int}CD34+ HSCs were 17.7/μL in the mid-dose group. More stem/progenitor cells were mobilized in the mid-dose group than in the high-dose group except for CD45^{Int}CD34+CD133+VEGFR2+ early endothelial progenitor cells.

Composition a Down-Regulates Pathways Associated with Inflammation and Allograft Rejection

RNA sequencing transcriptome studies of peripheral blood mononuclear cells (PBMC) showed no change in gene expression at different time points in the placebo treatment group over the measured 48-hour period, which spans one injection cycle. Significant differences in gene expression between placebo and 0.01 mL/kg COMPOSITION A or 0.02 mL/kg COMPOSITION A injection were observed (Table 5). Significant changes in gene expression occurred in the group treated with the mid-dose of COMPOSITION A (0.01 mL/kg) including 850 down-regulated genes and 1,049 up-regulated genes at 8 hours, 1,474 down-regulated genes and 825 up-regulated genes at 12 hours and 270 down-regulated genes and 18 up-regulated genes at 24 hours post-injection. In contrast to the middle dose group, high-dose of COMPOSITION A treatment (0.02 mL/kg) resulted in fewer changes in gene expression including 66 down-regulated genes and 25 up-regulated genes at 1 hour, 1 up-regulated gene at 3 hours, 7 down-regulated genes and 32 up-regulated genes at 8 hours, 484 down-regulated genes and 202 up-regulated genes at 12 hours, 202 down-regulated genes and 55 up-regulated genes at 24 hours, and 70 down-regulated genes and 8 up-regulated genes at 48 hours. To discover the biological pathways affected by different dosages of COMPOSITION A, Gene Set Enrichment Analysis (GSEA) was employed. We specifically studied the time point where the most genetic changes occurred, which was at 8, 12, 24 and 48 hours. Compared to the placebo group, 17, 24 and 19 down-regulated pathways were identified at 8, 12 and 24 hours in the mid-dose group (FIG. 5A), while 4, 9, 5 and 8 down-regulated pathways at 8, 12, 24 and 48 hours and 1 up-regulated pathway at 8, 24 and 48 hours were identified in the high-dose group (FIG. 5B). Totally, the GSEA of RNA sequencing data identified 31 down-regulated pathways in the mid-dose group, 14 down-regulated pathways and 1 up-regulated pathway in the high-dose group. Ten down-regulated pathways including TNF-alpha signaling via NFKB, TGF-beta signaling, allograft rejection, oxidative phosphorylation, coagulation, complement, inflammatory response, P53 pathway, apoptosis and IL-2 STAT5 signaling were consistently present at 8, 12 and 24 hours in the mid-dose group, while 4 pathways including MYC target V1, oxidative phosphorylation, allograft rejection and unfolded protein response were consistently down-regulated at 8, 12, 24 and/or 48 hours at the high-dose.

Considering the diversity of testing subjects, we have also compared gene expression at different time points to pre-dose. Compared to the pre-dose, 654 down-regulated genes and 1,038 up-regulated genes at 8 hours, 630 down-regulated genes and 225 up-regulated genes at 12 hours, 199 down-regulated genes and 9 up-regulated genes at 24 hours and 1 up-regulated gene at 48 hours were identified in the mid-dose group (Table S1). Fifteen down-regulated genes and 29 up-regulated genes at 8 hours, 132 down-regulated genes and 72 up-regulated genes at 12 hours and 78 down-regulated genes and 43 up-regulated genes at 24 hours were identified in the high-dose group (Table S1). Totally, the GSEA of RNA sequencing data identified 1 up-regulated pathway (Interferon alpha response) and 29 down-regulated pathways in the middle dose group (FIG. 6A), and 5 down-regulated pathways in the high dose group (FIG. 6B).

Venn diagram analysis of common down-regulated pathways between COMPOSITION A versus placebo and COMPOSITION A versus pre-dose revealed 27 common down-regulated pathways in the mid-dose group and 4 down-regulated pathways in the high-dose group (FIG. 7). Most down-regulated pathways (27/31) in the mid-dose group compared to the placebo group were also observed in the mid-dose group comparison with the pre-dose (27/29).

### Discussion

In this first-in-human study, COMPOSITION A was safe and well tolerated at doses up to 0.02 mL/kg QOD SC in the 3 multiple ascending dose cohorts in healthy subjects. There were no deaths, SEA or severe TEAE. Most TEAE were mild, and there was a dose-dependent increase in TEAE frequency; 68% of TEAEs (26/38) including 4 moderate TEAEs were observed in the high-dose cohort. These events were temporary and eventually, all were resolved within 24-48 hours. No clinically significant laboratory changes were observed besides the expected mobilization of different lineages of white blood cells.

Following multiple-dose administration, plasma plerixafor or tacrolimus Cₘₐₓ and AUC generally increased in an apparent dose-proportional manner across the dose range studied. COMPOSITION A PK parameters were comparable for single and repeated subcutaneous injection suggesting no time-dependent changes in PK in this study. The PK of both plerixafor and tacrolimus was dose-proportional and never reached toxic or immunosuppressive thresholds. The 0.01 ml/kg dose group demonstrated the desired tacrolimus range based on previous preclinical studies (1,2,4,5,10-13) and blood trough levels were less than 2 ng/ml on day 1 or 3 ng/mL on day 5 post-injection suggesting no immunosuppression. Tacrolimus levels below 2 ng/mL are generally below the LLQ in clinical laboratories and at or above 5 ng/ml are considered immunosuppressive (16).

The action of COMPOSITION A in animal models of human diseases is to mobilize bone marrow stem cells and immunoregulatory cells to the peripheral blood and recruit mobilized stem cells and immunoregulatory cells into the injured sites (1,2,4,5,10-13). In this study, COMPOSITION A is effective in mobilizing a variety of stem/progenitor cells and immune cells including CD4+Foxp3+ and CD8+Foxp3+ regulatory T cells, which is consistent with the findings in animal models. Interestingly, stem cell/immune cell mobilization was not dose-proportional in COMPOSITION A-treated subjects. Mid-dose COMPOSITION A dramatically mobilized bone marrow stem cells such as HSCs and CD4+ and CD8+ regulatory T cells, and peak CD45^{Int}CD34+HSCs were 43.86/μL blood (FIG. 4A) that is greater than reported results from a plerixafor trial (17). The normal dose of plerixafor (0.24 mg/kg) mobilizes 27.8 CD34+HSCs/μL at peak levels, while high dose plerixafor (0.48 mg/kg) mobilizes greater numbers of CD34+HSCs (peak CD34+: 32.2 c/μL). However, high-dose COMPOSITION A (plerixafor 0.48 mg/kg) mobilized stem cells moderately (peak CD34+HSCs:10.99/μL) without increasing regulatory T cells. Animal studies demonstrated that low-dose, but not high-dose, FK506 reacted synergistically with AMD3100 (plerixafor) in mobilizing stem cells and regulatory T cells (1,2) through activation of BMP signaling (14). Increasing FK506 levels may impact the synergism with AMD3100 and therefore reduce the efficacy of high-dose COMPOSITION A in mobilizing stem cells and regulatory T cells.

All cell populations returned to baseline levels before the next injection, consistent with no accumulative effect. In animal models of human diseases, mobilized stem cells and immunoregulatory cells are recruited into the injured organ/tissues and promoted tissue repair/regeneration (1,2,4,5,10-13). However, in healthy subjects, these mobilized cells stay in circulation for up to 24 hours and likely return to the bone marrow reservoir.

Foxp3+ regulatory T cells have a critical role in the maintenance of immune homeostasis, prevention of autoimmunity and induction of allograft tolerance (18). Expanding and/or infusion of Tregs can effectively prevent allograft rejection and cure autoimmune diseases without significant side effects (19). Indeed, their functions extend beyond immune surveillance to tissue homeostasis, including regulation of local and systemic metabolism, promotion of tissue repair and regeneration, and control of the proliferation, differentiation and fate of non-lymphoid cell progenitors (20). COMPOSITION A given at the mid-dose dramatically increased circulating CD4+Foxp3+ and CD8+Foxp3+ Tregs suggesting its immunomodulatory properties and that COMPOSITION A could be used as an immunoregulatory therapy in a variety of human diseases including autoimmune diseases and transplant rejection.

Circulating hematopoietic stem and progenitor cells (HSPCs) including CD34+ and CD34+CD133+ are rare, but changes in circulating HSPCs were reported to relate to the outcomes of several diseases in the cardiovascular system, lung, kidney and liver. Low CD34+ and CD34+CD133+ cell levels significantly and independently predicted the development or worsening of microangiopathy in diabetic patients (21) and a reduced CD34+ and CD34+CD133+ cell count independently predicted future events in patients with type 2 diabetes (22). Several studies have reported that patients with low levels of circulating HSPCs were at a significantly higher risk for future cardiovascular disease than were patients with higher cell levels (23-26) and a decrease in circulating HSPC counts during exercise is associated with worse prognosis and is a stronger factor in outcomes than the presence of stress-induced myocardial ischemia in patients with coronary artery disease (27). Similarly, circulating progenitor cells are reduced in patients with severe lung disease (28) and lower CD34+HSPCs were associated with a more than threefold higher risk of adverse outcome in COVID-19 patients. Reduction of HSPCs was a significant mediator of the admission of hyperglycemia on COVID-19 outcome, being responsible for 28% of its prognostic effect (29). Conversely, a higher number of CD34+ and CD34+CD133+HSPCs was inversely associated with all-cause and cardiovascular mortality (30). Infusion of CD34+ cells in patients with chronic kidney disease improved 1-year outcome (31) and CD34+ and CD133+ stem cells infusion has been used as a supportive treatment for end-stage liver disease with satisfactory tolerability (32). However, mobilization of HSPCs with plerixafor did not promote the healing of ischemic wounds and might exert adverse effects on wound healing in diabetic patients (33), and a similar result was reported in a mouse model of surgical wounds (2) suggesting the necessity of activating other pathways (10, 14). Activation of BMP signaling by FKBP12 ligands (low-dose tacrolimus) in combination with plerixafor exhibited a synergy in mobilizing and recruitment of stem cells into the injured sites and promoted diabetic wound healing (14). COMPOSITION A consistently increased circulating stem cells following multiple-ascending dose treatment (FIG. 4) indicating the potent stem cell mobilizing activity of COMPOSITION A and its potential as a regenerative therapy in patients with organ/tissue injury.

Notably, mobilizing stem cells and immunoregulatory T cells are associated with changes in gene expression of PBMCs. RNA sequencing of PBMCs and the gene set enrichment analysis revealed 27 common down-regulated pathways in the mid-dose cohort and 4 common-down regulated pathways in the high-dose cohort, compared to the placebo group or pre-dose (FIG. 7). Release from the bone marrow may cause stem cell and immune activation, however the multitude of genes and pathways influenced by COMPOSITION A was unexpected. Most of these 27 pathways in PBMC are related to proinflammatory and inflammatory response, complement and coagulation activation, T and B cell activation and function, metabolic reprogramming in T cells, oxidative stress, cell death and secreting inflammatory cytokines. Down-regulating 27 pathways suggest a number of potential activities of COMPOSITION A including anti-inflammation/allograft rejection (TNFalpha signaling via NFKB, inflammatory response, allograft rejection, IL-2 STAT5 signaling, IL-6 JAK/STAT3 signaling, oxidative phosphorylation, etc.), anti-ischemia/reperfusion injury (hypoxia, ROS pathway, P53 pathway, mTORC1 signaling pathway, apoptosis, etc.), anti-thrombosis (coagulation and complement pathways), anti-fibrosis (WNT Beta-catenin signaling, TGF Beta signaling) and anti-aging (mTORC1 signaling, IL-6 JAK/STAT3 signaling, inflammatory response, ROS pathway, unfolded protein response, etc.). Indeed, animal studies have demonstrated some of these activities in a variety of animal models of human diseases (1,2,4,5,10-13), especially anti-allograft rejection (1,4,5). It is worth mentioning that several pathways (i.e. MYC target V1, Kras signaling up, androgen response, peroxisome, unfolded protein response) down-regulated by COMPOSITION A are also related to cancer and decreasing expression of these pathways such as MYC have been considered as novel therapeutic strategies for cancer treatment (34).

Higher numbers of mobilized stem cells and immunoregulatory T cells in peripheral blood are correlated with more down-regulated pathways in PBMCs. However, the kinetics of gene expression changes did not correspond to kinetic changes of stem cells and/or immunoregulatory T cells. For example, circulating immune cells and stem cells fell back to placebo levels at 48 hours in the high-dose cohort but 6 down-regulated pathways remained. The mid-dose, not high-dose, mobilized more stem cells and immunoregulatory T cells and down-regulated more pathways. The peak levels of tacrolimus (2-3 ng/ml) in the middle dose cohort (FIG. 1C) are within the desired range based on preclinical studies. Down-regulating 27 pathways by the middle dose COMPOSITION A may be due to the synergy of plerixafor and low-dose tacrolimus. The mechanisms causing changes in particular gene expression by COMPOSITION A clearly warrant further investigation.

Over a decade of animal studies led to the development of COMPOSITION A. Due to its immunoregulatory and regenerative therapeutic properties discovered from animal studies and its safety in healthy subjects, COMPOSITION A is being tested in a phase II trial for efficacy and safety in severely, critically ill, COVID-19 patients who suffer from ARDS. In summary, multiple doses of COMPOSITION A up to 0.02 mL/kg (QAD, SC injection) were safe and well-tolerated in healthy participants in this study. The 0.01 mL/kg COMPOSITION A dose may be a promising and safe dosage for mobilizing bone marrow stem cells, immune regulatory T cells, and down-regulating pathways related to inflammation and other diseases. Further studies in phase II clinical trials are warranted to investigate the efficacy and safety of COMPOSITION A in patients with inflammatory disorders, wounds and organ transplantation.

Tables

TABLE 1
Demographic and Other Baseline Characteristics

| | | Cohort 1 | | Cohort 2 | | Cohort 3 | | | | | |
|---|---|---|---|---|---|---|---|---|---|---|---|
| | | Composition A | | Composition A | | Composition A | | Pooled | | | |
| | | 0.005 mL/kg | | 0.01 mL/kg | | 0.02 mL/kg | | Placebo | | Overall | |
| | | (N = 4) | | (N = 5) | | (N = 5) | | (N = 7) | | (N = 21) | |
| Parameter | Statistic | n (%) | | n (%) | | n (%) | | n (%) | | n (%) | |
| Age (years) | n | 4 | | 5 | | 5 | | 7 | | 21 | |
| | Mean | 33.5 | | 32.4 | | 36.2 | | 29.1 | | 32.4 | |
| | Min | 21 | | 26 | | 27 | | 20 | | 20 | |
| | Max | 42 | | 36 | | 44 | | 35 | | 44 | |
| Gender | | | | | | | | | | | |
| Female | n (%) | 3 | (75.0) | 3 | (60.0) | 3 | (60.0) | 5 | (71.4) | 14 | (66.7) |
| Male | n (%) | 1 | (25.0) | 2 | (40.0) | 2 | (40.0) | 2 | (28.6) | 7 | (33.3) |
| Race | | | | | | | | | | | |
| Asian | n (%) | 0 | | 0 | | 1 | (20.0) | 0 | | 1 | (4.8) |
| Black or African | n (%) | 0 | | 1 | (20.0) | 2 | (40.0) | 1 | (14.3) | 4 | (19.0) |
| American | | | | | | | | | | | |
| White | n (%) | 4 | (100.0) | 4 | (80.0) | 2 | (40.0) | 6 | (85.7) | 16 | (76.2) |
| Ethnicity | | | | | | | | | | | |
| Hispanic or | n (%) | 2 | (50.0) | 3 | (60.0) | 2 | (40.0) | 6 | (85.7) | 13 | (61.9) |
| Latino | | | | | | | | | | | |
| Not Hispanic or | n (%) | 2 | (50.0) | 2 | (40.0) | 3 | (60.0) | 1 | (14.3) | 8 | (38.1) |

| Latino | | | | | | |
|---|---|---|---|---|---|---|
| Height (cm) | n | 4 | 5 | 5 | 7 | 21 |
| | Mean | 169.98 | 168.14 | 164.46 | 164.11 | 166.27 |
| | Min | 155.5 | 158.8 | 153.2 | 158.0 | 153.2 |
| | Max | 185.6 | 176.0 | 175.5 | 172.6 | 185.6 |
| Weight (kg) | n | 4 | 5 | 5 | 7 | 21 |
| | Mean | 69.23 | 76.24 | 73.60 | 69.84 | 72.14 |
| | Min | 59.1 | 61.3 | 52.2 | 55.4 | 52.2 |
| | Max | 87.0 | 92.7 | 96.6 | 76.7 | 96.6 |
| BMI (kg/m²) | n | 4 | 5 | 5 | 7 | 21 |
| | Mean | 24.00 | 26.90 | 26.80 | 25.89 | 25.99 |
| | Min | 20.2 | 22.2 | 21.9 | 22.2 | 20.2 |
| | Max | 28.3 | 30.7 | 31.4 | 29.1 | 31.4 |

Abbreviations: BMI=body mass index: Max=maximum; Min=minimum; n=number of non-missing observations: N=number of subjects in respective category

TABLE 2
Summary of Treatment-Emergent Adverse Events by System Organ Class and Preferred Term (Safety Set)

| | Cohort 1 | | Cohort 2 | | Cohort 3 | | | | | |
|---|---|---|---|---|---|---|---|---|---|---|
| | Composition A | | Composition A | | Composition A | | Pooled | | | |
| | 0.005 mL/kg | | 0.01 mL/kg | | 0.02 mL/kg | | Placebo | | Overall | |
| | (N = 4) | | (N = 5) | | (N = 5) | | (N = 7) | | (N = 21) | |
| | n (%) | | n (%) | | n (%) | | n (%) | | n (%) | |
| System Organ | No. of | | No. of | | No. of | | No. of | | No. of | |
| Class | Subjects | No. of | Subjects | No. of | Subjects | No. of | Subjects | No. of | Subjects | No. of |
| Preferred Term | (%)^{a} | Events^{b} | (%)^{a} | Events^{b} | (%)^{a} | Events^{b} | (%)^{a} | Events^{b} | (%)^{a} | Events^{b} |
| Subjects with | 2 | 6 | 3 | 5 | 5 | 26 | 1 | 1 | 11 | 38 |
| any TEAE | (50.0) | | (60.0) | | (100.0) | | (14.3) | | (52.4) | |
| Gastrointestinal | 2 | 2 | 2 | 2 | 3 | 8 | 0 | 0 | 7 | 12 |
| disorders | (50.0) | | (40.0) | | (60.0) | | | | (33.3) | |
| Abdominal pain | 0 | 0 | 0 | 0 | 1 | 3 | 0 | 0 | 1 | 3 |
| | | | | | (20.0) | | | | (4.8) | |
| Diarrhea | 1 | 1 | 1 | 1 | 1 | 1 | 0 | 0 | 3 | 3 |
| | (25.0) | | (20.0) | | (20.0) | | | | (14.3) | |
| Dry mouth | 1 | 1 | 0 | 0 | 0 | 0 | 0 | 0 | 1 | 1 |
| | (25.0) | | | | | | | | (4.8) | |
| Nausea | 0 | 0 | 1 | 1 | 2 | 4 | 0 | 0 | 3 | 5 |
| | | | (20.0) | | (40.0) | | | | (14.3) | |
| General | 1 | 1 | 2 | 2 | 5 | 12 | 1 | 1 | 9 | 16 |
| disorders and | (25.0) | | (40.0) | | (100.0) | | (14.3) | | (42.9) | |
| administration | | | | | | | | | | |
| site conditions | | | | | | | | | | |
| Influenza | 0 | 0 | 0 | 0 | 1 | 1 | 0 | 0 | 1 | 1 |
| like illness | | | | | (20.0) | | | | (4.8) | |
| Injection site | 0 | 0 | 0 | 0 | 1 | 1 | 0 | 0 | 1 | 1 |
| erythema | | | | | (20.0) | | | | (4.8) | |
| Injection site | 0 | 0 | 0 | 0 | 1 | 1 | 0 | 0 | 1 | 1 |
| hemorrhage | | | | | (20.0) | | | | (4.8) | |
| Injection site | 1 | 1 | 2 | 2 | 4 | 8 | 1 | 1 | 8 | 12 |
| pain | (25.0) | | (40.0) | | (80.0) | | (14.3) | | (38.1) | |
| Injection site | 0 | 0 | 0 | 0 | 1 | 1 | 0 | 0 | 1 | 1 |
| swelling | | | | | (20.0) | | | | (4.8) | |
| Infections and | 0 | 0 | 0 | 0 | 1 | 1 | 0 | 0 | 1 | 1 |
| infestations | | | | | (20.0) | | | | (4.8) | |
| Vulvovaginal | 0 | 0 | 0 | 0 | 1 | 1 | 0 | 0 | 1 | 1 |
| candidiasis | | | | | (20.0) | | | | (4.8) | |
| Musculoskeletal | 0 | 0 | 0 | 0 | 1 | 1 | 0 | 0 | 1 | 1 |
| and connective | | | | | (20.0) | | | | (4.8) | |
| tissue disorders | | | | | | | | | | |
| Musculoskeletal | 0 | 0 | 0 | 0 | 1 | 1 | 0 | 0 | 1 | 1 |
| chest pain | | | | | (20.0) | | | | (4.8) | |
| Nervous system | 2 | 3 | 1 | 1 | 2 | 3 | 0 | 0 | 5 | 7 |
| disorders | (50.0) | | (20.0) | | (40.0) | | | | (23.8) | |
| Dizziness | 0 | 0 | 0 | 0 | 1 | 1 | 0 | 0 | 1 | 1 |
| | | | | | (20.0) | | | | (4.8) | |
| Headache | 2 | 3 | 0 | 0 | 1 | 1 | 0 | 0 | 3 | 4 |
| | (50.0) | | | | (20.0) | | | | (14.3) | |
| Paraesthesia | 0 | 0 | 1 | 1 | 0 | 0 | 0 | 0 | 1 | 1 |
| | | | (20.0) | | | | | | (4.8) | |
| Somnolence | 0 | 0 | 0 | 0 | 1 | 1 | 0 | 0 | 1 | 1 |
| | | | | | (20.0) | | | | (4.8) | |
| Psychiatric | 0 | 0 | 0 | 0 | 1 | 1 | 0 | 0 | 1 | 1 |
| disorders | | | | | (20.0) | | | | (4.8) | |
| Insomnia | 0 | 0 | 0 | 0 | 1 | 1 | 0 | 0 | 1 | 1 |
| | | | | | (20.0) | | | | (4.8) | |

Abbreviations: n. N. or No.=number; TEAE=treatment-emergent adverse event

^{a} Subject will be counted only once in each category for more than 1 event.

^{b} Subject can be represented more than once.

TABLE 3
Summary of Plerixafor Pharmacokinetic Parameters on Days 1 and 5

| | | Cohort 1 | | Cohort 2 | | Cohort 3 | |
|---|---|---|---|---|---|---|---|
| | | Composition A | | Composition A | | Composition A | |
| | | 0.005 mL/kg | | 0.01 mL/kg | | 0.02 mL/kg | |
| Parameter | | Day 1 | Day 5 | Day 1 | Day 5 | Day 1 | Day 5 |
| (unit) | Statistic | (N = 4) | (N = 4) | (N = 5) | (N = 4) | (N = 4) | (N = 4) |
| Cₘₐₓ | n | 4 | 4 | 4 | 4 | 4 | 4 |
| (ng/mL) | GM | 274 | 302 | 601 | 613 | 947 | 1115 |
| | GeoCV % | 15.3 | 11.0 | 11.9 | 21.0 | 8.65 | 7.49 |
| Tₘₐₓ | n | 4 | 4 | 4 | 4 | 4 | 64 |
| (h) | Median | 2.00 | 1.00 | 3.00 | 1.00 | 2.00 | 1.00 |
| | Min, Max | 1.00, 3.00 | 1.00, 3.00 | 1.00, 3.00 | 2.01, 3.05 | 1.00, 3.00 | 1.00, 3.00 |
| AUCₜₐᵤ | n | 2 | 3 | 4 | 4 | 4 | 4 |
| (ng*h/mL) | GM | 1598 | 1846 | 4171 | 4336 | 7325 | 8916 |
| | GeoCV % | 1.69 | 14.9 | 5.48 | 13.5 | 19.1 | 22.9 |
| t_{1/2} | n | 2 | 3 | 4 | 4 | 4 | 4 |
| (h) | GM | 3.29 | 4.56 | 4.26 | 4.56 | 5.01 | 6.06 |
| | GeoCV % | 1.16 | 3.38 | 13.3 | 3.38 | 17.8 | 28.8 |

Abbreviations: AUC=area under the plasma concentration time curve; AUCta=AUC over 1 dosing interval; Cₘₐₓ=maximum observed plasma concentration; GeoCV %=geometric percent coefficient of variation; GM=geometric mean; max=maximum; min=minimum; n=number of non-missing observations; N=number of subjects in respective category; PK=pharmacokinetic; t_{1/2}=terminal elimination half-life; Tₘₐₓ=time of maximum observed plasma concentration.

TABLE 4
Summary of Tacrolimus Pharmacokinetic Parameters on Days 1 and 5

| | | Cohort 1 | | Cohort 2 | | Cohort 3 | |
|---|---|---|---|---|---|---|---|
| | | Composition A | | Composition A | | Composition A | |
| | | 0.005 mL/kg | | 0.01 mL/kg | | 0.02 mL/kg | |
| Parameter | | Day 1 | Day 5 | Day 1 | Day 5 | Day 1 | Day 5 |
| (unit) | Statistic | (N = 4) | (N = 4) | (N = 5) | (N = 4) | (N = 4) | (N = 4) |
| Cₘₐₓ | n | 4 | 4 | 5 | 4 | 4 | 4 |
| (ng/mL) | GM | 1.47 | 2.09 | 3.15 | 5.37 | 4.49 | 7.91 |
| | GeoCV % | 34.7 | 11.3 | 18.0 | 11.2 | 28.8 | 14.2 |
| Tₘₐₓ | n | 4 | 4 | 5 | 4 | 4 | 64 |
| (h) | Median | 7.50 | 10.00 | 12.00 | 5.50 | 8.01 | 2.00 |
| | Min, Max | 3.00, 12.00 | 1.00, 12.03 | 1.00, 12.02 | 1.00, 12.00 | 3.00, 24.00 | 1.00, 24.00 |
| AUCₜₐᵤ | n | 4 | 4 | 5 | 4 | 4 | 4 |
| (ng*h/mL) | GM | 46.4 | 70.1 | 105 | 171 | 152 | 237 |
| | GeoCV % | 10.8 | 13.1 | 14.7 | 19.7 | 23.0 | 28.0 |
| t_{1/2} | n | 2 | 2 | 2 | 3 | 3 | 3 |
| (h) | GM | 23.8 | 24.9 | 37.8 | 29.5 | 36.4 | 30.6 |
| | GeoCV % | 25.2 | 6.11 | 69.2 | 43.9 | 33.8 | 22.5 |
| C_{trough} | n | 4 | 4 | 4 | 4 | 4 | 4 |
| (ng/mL) | GM | 0.57 | 0.88 | 1.45 | 1.89 | 2.12 | 2.94 |
| | GeoCV % | 26.07 | 26.19 | 26.80 | 41.56 | 38.77 | 47.25 |

Abbreviations: AUC=area under the plasma concentration time curve; AUCia=AUC over 1 dosing interval; Cₘₐₓ=maximum observed plasma concentration; GeoCV %=geometric percent coefficient of variation; GM=geometric mean; max=maximum; min=minimum; n=number of non-missing observations; N=number of subjects in respective category; PK=pharmacokinetic; t_{1/2}=terminal elimination half-life; Tₘₐₓ=time of maximum observed plasma concentration; C_{trough}=trough concentration.

TABLE 5
Changes in Gene Expression and Molecular Pathways - Composition A versus Placebo

| | Down-regulated genes | | | | | | Up-regulated genes | | | | | |
|---|---|---|---|---|---|---|---|---|---|---|---|---|
| Dosage | 1 h | 3 h | 8 h | 12 h | 24 h | 48 h | 1 h | 3 h | 8 h | 12 h | 24 h | 48 h |
| Placebo | 0 | 0 | 0 | 0 | 0 | 0 | 0 | 0 | 0 | 0 | 0 | 0 |
| 0.005 mL/kg | 5 | 0 | 0 | 0 | 2 | 0 | 1 | 1 | 1 | 0 | 0 | 0 |
| 0.01 mL/kg | 0 | 0 | 850 | 1474 | 270 | 0 | 0 | 0 | 1494 | 825 | 18 | 0 |
| 0.02 mL/kg | 66 | 0 | 7 | 484 | 202 | 70 | 25 | 1 | 32 | 202 | 55 | 8 |

| | Down-regulated pathways | | | | | | Up-regulated pathways | | | | | |
|---|---|---|---|---|---|---|---|---|---|---|---|---|
| Dosage | 1 h | 3 h | 8 h | 12 h | 24 h | 48 h | 1 h | 3 h | 8 h | 12 h | 24 h | 48 h |
| Placebo | 0 | 0 | 0 | 0 | 0 | 0 | 0 | 0 | 0 | 0 | 0 | 0 |
| 0.005 mL/kg | 0 | 0 | 0 | 0 | 0 | 0 | 0 | 0 | 0 | 0 | 0 | 0 |
| 0.01 mL/kg | 0 | 0 | 17 | 24 | 19 | 0 | 0 | 0 | 0 | 0 | 0 | 0 |
| 0.02 mL/kg | 0 | 0 | 4 | 9 | 5 | 8 | 0 | 0 | 1 | 0 | 1 | 1 |

### Supplemental Materials S1, Table S1-S3.

### Supplemental Text S1.

### Inclusion Criteria

Subjects who meet the following criteria will be considered eligible to participate in the clinical study:

1. Subject voluntarily agrees to participate in this study and signs an Institutional Review Board (IRB)-approved informed consent before performing any screening visit procedures.

2. Males and females between 18 to 45 years of age, inclusive, at the time of signing the ICF.

3. Subjects who test negative for SARS-COV-2 by real-time transcription polymerase chain reaction in the respiratory tract (nasopharyngeal [NP] swab) within the previous 96 hours.

4. Nonsmokers (or other nicotine use) as determined by history (no nicotine use over the past 6 months) and by urine cotinine concentration (<200 ng/mL) at the Screening Visit and prior to admission.

5. Generally, in good health with no clinically significant abnormalities as determined by medical history, physical examination, 12-lead ECG and clinical laboratory tests.

6. The following applies to female subjects: Non-pregnant, non-lactating females of childbearing potential who agree to use medically acceptable forms of birth control (hormonal contraception, abstinence, diaphragm with spermicide, condom with spermicide or intrauterine device) from the Screening Visit until the End-of-study Visit.

7. Body mass index (BMI) between 18.8 and 32.0 kg/m2, inclusive, at the Screening Visit.

8. A fasting blood glucose level≤125 mg/dL (6.9 mmol/L), at the Screening Visit.

### Exclusion Criteria

1. Laboratory-confirmation of positive SARS-COV-2 by real-time polymerase chain reaction in the respiratory tract (NP swab, tracheal aspirate, BAL)≤96 hours prior to randomization.

2. Has any concurrent disease or condition that, in the opinion of the Principal Investigator, would make the subject unsuitable for participation in the clinical study such as:
a. Skin condition or disease (e.g., Stevens-Johnson syndrome).
b. Hypertension defined as >140 mmHg systolic blood pressure and >95 mmHg diastolic blood pressure.
c. High blood potassium (hyperkalemia) defined baseline serum potassium >5.0 to 5.5 mEq/L (milliequivalent).
d. Torsades de Pointes or currently taking medication that prolongs QT interval.
e. Hematologic disorder (e.g. anemia or leukemia).
f. Type I or Type 2 diabetes mellitus is defined as a fasting blood glucose level >126 mg/dL (7.8 mmol/L), at the Screening Visit.

3. Subject has a clinically significant history or evidence of cardiovascular, respiratory, hepatic, renal, gastrointestinal, endocrine, neurological, immunological or psychiatric disorder(s) as determined by the Principal Investigator or designee.

4. History of splenomegaly (spleen weighing >750 g).

5. History of cancer or thrombocytopenia (platelet count <100,000/μL) or thrombocythemia (platelet count >500,000/μL).

6. Known family history of long QT syndrome (Torsades de Pointes) or currently taking medication that prolongs QT interval.

7. Female subjects who are pregnant or breastfeeding or planning to breastfeed at any time through 90 days after the last dose of IP.

8. Any disorder that would interfere with the absorption, distribution, metabolism or excretion of drugs.

9. Received a vaccination (including influenza) administered 30 days or less prior to first treatment/randomization or has any planned vaccinations during the treatment period.

10. Has the following liver function levels: Serum ALP or BIL>1.5 ULN or ALT or AST>ULN at either screening or admission. Only 1 repeat assessment is allowed on each occasion.

11. History of alcohol and/or illicit drug abuse within 2 years of entry.

12. Positive test for Hepatitis B surface antigen (HBsAg), Hepatitis C antibody or human immunodeficiency virus (HIV) antibody.

13. Is unwilling to avoid use of alcohol or alcohol-containing foods, medications or beverages, within 48 hours prior to screening and for the duration of the study.

14. Is unable to abstain from smoking (or other nicotine use) from screening and for the duration of the study.

15. Has a positive urine test for ethanol at the Screening Visit or admission.

16. Has a positive urine drug test (e.g., cocaine, amphetamines, barbiturates, opiates, benzodiazepines, cannabinoids) at the Screening Visit or admission.

17. Has donated blood (>500 mL) or blood products within 2 months (56 days) prior to admission.

18. Has used an investigational drug within 30 days prior to Screening.

19. History of hypersensitivity to Composition A (plerixafor [AMD3100, 24 mg/mL]) and tacrolimus [FK506, 0.5 mg/mL]) or any of the excipients or to medicinal products with similar chemical structures.

20. Unable to understand the protocol requirements, instructions and study-related restrictions, the nature, scope and possible consequences of the clinical study.

21. Unlikely to comply with the protocol requirements, instructions and study-related restrictions; e.g., uncooperative attitude, inability to return for follow-up visits and improbability of completing the clinical study.

22. Previously enrolled in this clinical study.

23. Vulnerable subjects are defined as individuals whose willingness to volunteer in a clinical study may be unduly influenced by the expectation, whether justified or not, of benefits associated with participation or of a retaliatory response from senior members of a hierarchy in case of refusal to participate (e.g., persons in detention, minors and those incapable of giving consent).

TABLE S1
Changes in Gene expression and Molecular
Pathways - Composition A versus Pre-dose.

| | Down-regulated genes | | | | | | Up-regulated genes | | | | | |
|---|---|---|---|---|---|---|---|---|---|---|---|---|
| Dosage | 1 h | 3 h | 8 h | 12 h | 24 h | 48 h | 1 h | 3 h | 8 h | 12 h | 24 h | 48 h |
| Placebo | 0 | 0 | 0 | 0 | 0 | 0 | 0 | 0 | 0 | 0 | 0 | 0 |
| 0.005 mL/kg | 5 | 0 | 0 | 0 | 2 | 0 | 1 | 0 | 0 | 0 | 0 | 0 |
| 0.01 mL/kg | 0 | 0 | 654 | 630 | 199 | 0 | 0 | 2 | 1038 | 225 | 9 | 1 |
| 0.02 mL/kg | 0 | 0 | 15 | 132 | 78 | 0 | 0 | 0 | 29 | 72 | 43 | 0 |

| | Down-regulated pathways | | | | | | Up-regulated pathways | | | | | |
|---|---|---|---|---|---|---|---|---|---|---|---|---|
| Dosage | 1 h | 3 h | 8 h | 12 h | 24 h | 48 h | 1 h | 3 h | 8 h | 12 h | 24 h | 48 h |
| Placebo | 0 | 0 | 0 | 0 | 0 | 0 | 0 | 0 | 0 | 0 | 0 | 0 |
| 0.005 mL/kg | 0 | 0 | 0 | 0 | 0 | 0 | 0 | 0 | 0 | 0 | 0 | 0 |
| 0.01 mL/kg | 0 | 0 | 20 | 19 | 19 | 0 | 0 | 0 | 0 | 1 | 0 | 0 |
| 0.02 mL/kg | 0 | 0 | 0 | 4 | 4 | 0 | 0 | 0 | 0 | 0 | 0 | 0 |

TABLE S2
Differential Regulated Common Pathways by Composition A:
high dose (0.02 mL/kg) versus normal dose (0.01 mL/kg).

Hours post Composition
A injection

| | | | | |
|---|---|---|---|---|
| 0.02 mL/kg versus 0.01 mL/kg | 8 | 12 | 24 | 48 |

| | |
|---|---|
| Down regulated pathways (2) | |
| MYC Targets V1 | yes |
| MYC Targets V2 | yes |
| Up regulated pathways (5) | |
| TNFA Signaling via NFKB | yes |
| Inflammatory response | yes |
| Interferon Gamma Response | yes |
| Hypoxia | yes |
| Complement | yes |

TABLE S3
The list of antibodies for flow cytometry

| | Fluoro- | | |
|---|---|---|---|
| Antibody | chrome | Company | Cat# |
| Mouse Anti-Human CD4 | APC -H7 | BD | 560158 |
| Mouse Anti-Human CD8 | BUV805 | BD | 749366 |
| Mouse Anti-Human CD19 | BUV737 | BD | 741829 |
| Mouse Anti-Human CD56 | BV785 | Biolegend | 362550 |
| Mouse Anti-Human CD16 | PE | BD | 555407 |
| Mouse Anti-Human | BB515 | BD | 564552 |
| CD45RA | | | |
| Rat Anti-Human CCR7 | BV605 | BD | 563711 |
| Mouse Anti-Human CD25 | BV510 | BD | 563351 |
| Mouse Anti-Human | AF647 | BD | 560905 |
| CD127 | | | |
| Mouse Anti-Human CD3 | BV711 | BD | 563724 |
| Rat Anti-Human FOXP3 | eFluor450 | Thermofisher | 48-5773-82 |
| Live/Dead | Fixable Red | Thermofisher | L23102 |
| Mouse Anti-Human CD45 | PE/Dazzle | Biolegend | 982308 |
| | 594 | | |
| Mouse Anti-Human | PE/ | Biolegend | 372810 |
| CD133 | Cyanine 7 | | |
| Mouse Anti-Human CD31 | BV605 | BD | 745119 |
| Mouse Anti-Human CD34 | APC | BD | 345804 |
| Mouse Anti-Human | PE | Biolegend | 359904 |
| VEGFR2 | | | |
| Rat Anti-Human SSEA3 | PerCP- | BD | 561564 |
| | Cy5.5 | | |
| Mouse Anti-Human CD38 | BV711 | BD | 563965 |
| Mouse Anti-Human | BB515 | BD | 564552 |
| CD45RA | | | |
| Mouse Anti-Human CD90 | BV510 | BD | 563070 |
| Live/Dead | Fixable | Thermofisher | L23105 |
| | Blue | | |
| Counting Beads | N/A | Bangs | 580 |
| | | Laboratories | |

### Example 2—Methods for Treating Amyotrophic Lateral Sclerosis (ALS)

MRG-001 is a fixed dose combination drug which includes plerixafor and low-dose tacrolimus. Plerixafor (AMD3100) is a CXCR4 antagonist approved by the FDA in 2008 as a therapy whose purpose is to mobilize CD34+ hematopoietic stem cells (HSCs) into the peripheral blood for collection and subsequent autologous or allogeneic transplantation in patients with non-Hodgkin's lymphoma or multiple myeloma. Tacrolimus (FK-506) was approved by the FDA in 1994 to prevent organ rejection in patients receiving allogeneic organ transplants.

MRG-001 action involves the unexpected synergistic effects of AMD3100 and low-dose FK506 (AF) in mobilizing and recruiting stem cells and immunoregulatory cells, which the inventors discovered serendipitously and unexpectedly in animal models of organ transplantation. AMD3100 mobilizes bone marrow stem cells (for example, CD34+ and CD133+) and other immune cells, including Foxp3+ regulatory T cells, into the vascular circulation through blocking the interaction between CXCR4 and SDF1. Low-dose, non-immunosuppressive FK506 (less than one tenth the immunosuppressive dose) not only activates stem cell transcript factors via activation of (Bone Morphogenic Protein) BMP signaling but also helps to recruit these mobilized cells into the injured sites through activating M2 macrophages which produce SDF-1/HGF. The BMP pathway may induce proliferation and differentiation of stem cells. For the MRG-001 pharmacologic effect, these agents do not act alone but through their combined effect on bone marrow cells and the site of injury. In the injured area, stem cells recruited from the vasculature promote tissue repair/regeneration, and regulatory T cells and M2 macrophages can create a local environment of certain natural processes that favors tissue regeneration, modulates inflammatory responses, and prevents fibrotic scar formation. The inventors' studies demonstrated that the synergistic effect of AF combination in immunoregulation and tissue repair is not established through calcineurin-dependent immunosuppression but rather because of the ability of the low, but not immunosuppressive, dose of FK506 to activate the bone morphogenic pathway (BMP) pathway.

The combination of two drugs (plerixafor and non-immunosuppressive dose tacrolimus), now designated as MRG-001, enabled long-term liver or kidney allograft survival with just short-term treatment and freedom from immunosuppression in otherwise strongly rejecting rat strain combinations and in maximally immunologically mismatched swine. This acceptance was associated with allograft chimerism (Host repopulation) and local downregulation of the immune response through mobilizing and recruiting bone marrow-derived stem cells and immunoregulatory T cells. In addition to the allograft chimerism, stem cell mobilization and homing to damaged tissue due to the combination drug has, remarkably, proven to be safe and effective for skin wound healing, treating inflammatory bowel disease (IBD), preventing post-surgical peritoneal adhesions, promoting liver regeneration, improving wound healing and microvasculature in severe diabetes and prolonging skin allograft survival and in-situ skin repair and regeneration and healing in a variety of animal models of human diseases (unpublished data) in rodents and, in some cases, pigs and monkeys. Lineage tracing and bone marrow transplantation demonstrated the role of bone marrow derived CD133 stem cells in enhanced capillary and hair follicle neogenesis. MRG-001 was tested in a mouse model of acute respiratory distress syndrome (ARDS) induced by intra-tracheal instillation of Streptococcus pneumoniae and showed that MRG-001 ameliorated local/systemic inflammation, promoted regeneration of damaged bronchial epithelium through mobilizing/recruiting bone marrow CD133+ stem cells, Foxp3+ Tregs and M2 macrophages and reduced mortality. MRG-001 was tested in a GFP (green fluorescence protein) bone marrow transplanted rat model of contusive spinal cord injury and demonstrated that MRG-001 treatment mobilized and recruited bone marrow stem cells (GFP+) into the injured spinal cord, these stem cells repaired/regenerated damaged spinal cord tissues and improved the locomotor function.

The phase I trial confirms the favorable safety profiles and the efficacy of MRG-001 in mobilizing stem and regulatory T cells and down regulating 31 pathways related to inflammation. In this phase I, first-in-human study three cohorts receive subcutaneous MRG-001 or placebo, every other day for 5 days. Fourteen subjects received MRG-001 and seven received a placebo. MRG-001 is safe over the selected dose range. There are no clinically significant laboratory changes, vital signs or ECG abnormalities. Overall, 11 subjects (52.4%) reported 38 treatment-emergent adverse events (TEAE) after study drug treatment; 2 subjects (50.0%) reported 6 TEAE after treatment with MRG-001 0.005 ml/kg, 3 subjects (60.0%) reported 5 TEAEs after treatment with MRG-001 0.01 mL/kg, 5 subjects (100.0%) reported 26 TEAE after treatment with MRG-001 0.02 mL/kg, and 1 subject (14.3%) reported 1 TEAE after treatment with placebo. All TEAE were considered mild (n=34) or moderate (n=4) in severity; there were no serious TEAE. The majority of TEAE were definitely (9 subjects [42.9%], 15 TEAE), probably (2 subjects [9.5%], 2 TEAE), or possibly (7 subjects 33.3 [%], 16 TEAE) related.

In the MRG-001 0.005 mL/kg group (n=4), two subjects (50.0%) reported six TEAE including diarrhea, dry mouth, injection site pain, and headache. In the MRG-001 0.01 mL/kg group (n=5), three subjects (60.0%) reported five TEAE including diarrhea, nausea, injection site pain, and paresthesia. In the MRG001 0.02 mL/kg group (n=5), 5 subjects (100.0%) reported 26 TEAE, including abdominal pain, diarrhea, nausea, influenza like illness, injection site erythema, injection site hemorrhage, injection site pain, injection site swelling, vulvovaginal candidiasis, musculoskeletal chest pain, dizziness, headache, somnolence, and insomnia.

After SC administration of a single dose on day 1 and after multiple doses to day 5 of MRG-001 0.005, 0.01, and 0.02 mL/kg (24 mg plerixafor plus 0.5 mg tacrolimus per mL), the Cmax and AUC for both plerixafor and tacrolimus increased with increasing dose in what seems to be a dose-proportional manner. The t½ for plerixafor was short for all doses (3-6 h) and longer for tacrolimus (24-38 h). The lowest dose and the intermediate dose of MRG-001, demonstrated mean tacrolimus trough levels of 0.8 ng/ml and 1.8 ng/mL respectively. This confirmed that the 0.005 ml/kg and 0.01 mL/kg MRG-001 dosing does not lead to tacrolimus that can cause immunosuppression. For clinical immunosuppression to occur, trough concentration between 5-15 ng/ml are required, in addition to other immunosuppressive therapies. For all clinical trials, we are proposing to use dosages equal or lower to 0.01 mL/kg to minimize the potential for immunosuppression.

The mid-dose (0.01 mL/kg) group demonstrates the most significant white blood cell, stem cell, and immunoregulatory cell mobilization. In the mid-dose group, CD3+CD4+Foxp3+Tregs increased 3-, 5-, and 10-fold at 1, 3, and 8 h and remained at higher levels (3-fold) at 24 h after the first mid dose, while CD3+CD8+Foxp3+ Tregs increased 10- and 30-fold at 1 and 3 h and decreased back to baseline levels at 24 h. However, neither CD4+Foxp3+ nor CD8+Foxp3+ Tregs were increased in the high-dose group.

In the mid-dose group, after administration of MRG-001, CD45^{Int}CD34+, and CD45^{Int}CD34+CD133+ hematopoietic stem cells (HSCs) and CD45^{Int}CD34+CD133+CD31+ endothelial progenitor cells (EPCs) in peripheral blood increased at 1 h, reached peak levels at 12 h, remained at higher levels at 24 h, and decreased back to placebo levels at 48 h. At 12 h, these stem/progenitor cells increased approximately 15- to 17-fold from baseline levels (pre-dose) and the peak CD45^{Int}CD34+HSCs were 43.86/μL blood. CD45^{Int}CD34+CD90+ multipotent HSCs with long-term multilineage engraftment potential increased approximately 7-, 18-, and 26-fold from baseline levels at 3, 8, and 12 h. Compared with the placebo group, CD45^{Int}CD34+CD133+VEGFR2+ early EPCs increased approximately 3-, 28-, and 60-fold at 1, 3, and 24 h, while SSEA3-expressing HSCs (CD45^{Int}CD34+SSEA3+) also increased 3-, 8-, and 12-fold at 1, 3, and 8 h. In the high-dose group (0.02 mL/kg), stem cell populations were increased in a similar pattern but to a moderate degree, and the peak CD45^{Int}CD34+HSCs were 11/μL blood.

RNA sequencing transcriptome studies of peripheral blood mononuclear cells (PBMC) showed no change in gene expression at different time points in the placebo treatment group over the measured 48-h period, which spans one injection cycle. Significant changes in gene expression occurred in the group treated with the mid-dose of MRG-001 (0.01 mL/kg) including 850 down-regulated genes and 1,049 upregulated genes at 8 h, 1,474 down-regulated genes and 825 up-regulated genes at 12 h, and 270 down-regulated genes and 18 up-regulated genes at 24 h after injection. Gene set enrichment analysis (GSEA) of RNA sequencing data identified 31 down-regulated pathways in the mid-dose group (scc FIGS. 5A, 5B, 5C and 5D) and 14 down-regulated pathways and 1 up-regulated pathway in the high-dose group.

Ten down-regulated pathways including tumor necrosis factor (TNF)-alpha signaling via nuclear factor (NF)-kB, transforming growth factor (TGF)-beta signaling, allograft rejection, oxidative phosphorylation, coagulation, complement, inflammatory response, P53 pathway, apoptosis, and IL-2 STAT5 signaling were consistently present at 8, 12, and 24 h in the mid-dose group, while four pathways, including MYC target VI, oxidative phosphorylation, allograft rejection, and unfolded protein response, were consistently down-regulated at 8, 12, 24, and/or 48 h at the high dose.

The mechanisms underlying neurodegeneration in ALS are multifactorial and mediated through a complex interplay of molecular and genetic pathways. Specific pathways involved are increased generation of reactive oxygen species (ROS), glutamate excitotoxicity, mitochondrial dysfunction, axonal transport dysfunction and accumulation of cytoplasmic protein aggregates consisting of SOD1, TDP-43 and FUS. Activation of astrocytes and microglia results in secretion of proinflammatory cytokines, leading to neuroinflammation and motor neuron degeneration18. 19. The inventors found attenuating ALS pathogenesis (including ER stress/Oxidative stress/Mitochondrial dysfunction, reactive astrocytes/activated M1 microglia, neuroinflammation/systemic inflammation, and neurodegeneration/muscle denervation), protecting neuron/muscle from injury and healing or regenerating damaged motor neurons may prove therapeutically useful for patients with ALS.

In addition to the favorable safety profile for multiple-dose regimens, MRG-001 exhibits immunoregulatory and regenerative properties in a variety of animal models of human diseases including spinal cord injury, down-regulates 31 pathways related to inflammation/oxidative stress and mobilizes stem and immunoregulatory T cells in human.

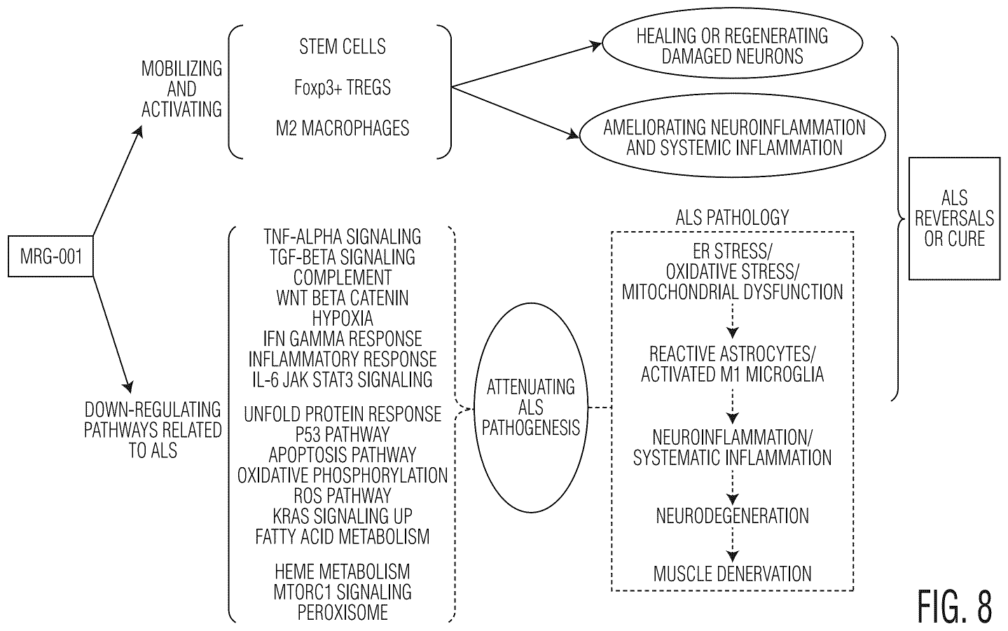

As shown in FIG. 8, MRG-001 can mobilize stem and immunoregulatory T cells, active M2 macrophages, and down-regulate pathways related to pathogenesis of ALS. 18 out of 31 down-regulated pathways (FIG. 8) by MRG-001 are related to pathogenesis of neurodegenerations and ALS, including neuroinflammation related pathways: TNF-alpha Signaling, TGF-beta Signaling, Complement, WNT Beta Catenin, Hypoxia, IFN Gamma Response, Inflammatory response, IL-6 JAK Stat3 signaling and neurodegeneration related pathways: Unfold Protein Response, P53 pathway, Apoptosis pathway, Oxidative Phosphorylation, ROS pathway, KRAS signaling Up, Fatty Acid Metabolism, Heme metabolism, MTORC1 Signaling and Peroxisome. It's unclear how MRG-001 down-regulated these pathways, but at least in part due to the synergism of AMD3100 and low-dose FK506. Both active pharmaceutical ingredients (APIs) are small molecules that can cross the blood-brain barrier (BBB) in vivo, thus likely exert effects on the brain/central nervous system directly.

MRG-001 may ameliorate systemic/neuroinflammation in ALS. Systemic inflammation may be important to ALS pathogenesis. A panel of five cytokines (IL-2, IL-6, IL-10, IFN-gamma, and TNF-alpha) were increased in plasma samples of patients (n=79) compared with matched healthy controls (n=79) (p<0.0001), with IL-6 having the highest median concentration (10.11 pg/ml) in the ALS group. In animal models of allograft rejection or acute lung injury, MRG-001 can reduce blood levels of these inflammatory cytokines including TNF-alpha and IL-6. In addition, MRG-001 can also promote macrophage polarization toward to an M2 phenotype to protect tissue from the excessive injury or promote tissue repair/regeneration. Microglia are the only macrophage population in the central nervous system (CNS) parenchyma, where they can interact with neurons, astrocytes and other CNS components. Oxidative stress activated pro-inflammatory MI microglia play a critical role in neuroinflammation in ALS. Activating M2 macrophages (microglia) by MRG-001 may ameliorate neuroinflammation via inactivating both MI microglia and reactive astrocytes.

MRG-001 mobilizes immunoregulatory T cells (Tregs) that may have regenerative capacity. ALS patients have dysregulated Tregs, the greater the clinically assessed disease burden or the more rapidly progressing the patient, the greater the Treg dysfunction. Expansion of the effector Treg population in the SODIG93A mice was associated with a significant slowing of disease progression, which was accompanied by an increase in survival time. There is strong evidence for blood-brain and blood-spinal cord barrier (BBB. BSCB) dysfunction at the early stages of many neurodegenerative diseases (NDDs), including amyotrophic lateral sclerosis (ALS). Migration of Tregs across of the dysfunctional BBB has been reported and Tregs mainly migrate through interactions of the LFA-I and CCR6 receptor. Next to the peripheral effects of Tregs, once migrated through the BBB. they can perform their suppressive properties in the CNS. These findings establish a neuroprotective effect of Tregs, possibly mediated by suppression of toxic neuroinflammation in the central nervous system. Interestingly, Tregs also exhibit reparative properties such as remyelination, mediated by growth-regulatory protein cellular communication network factor 3 (CCN3), and neural stem cell proliferation. Autologous infusion of expanded Treg cells in a small patient cohort slowed disease progression. In contrast to infusion of expanded Tregs, MRG-001 can dramatically increase both CD4+ and CD8+Foxp3+ Tregs that provides an alternative opportunity for treating ALS.

MRG-001 mobilizes a variety of bone marrow stem cells that offer the unique opportunity to simultaneously target multiple dysregulated pathways while providing CNS neurotrophic support. Stem cell therapy in ALS disease is based on “neighborhood theory”, where the transplanted cells secrete neuroprotective substances that limit the process of neurodegeneration. Transplanted stem cells, also, differentiate into astrocytes and microglia, or into other neurons, which connect with the affected motor neurons. It is important for stem cells to bypass the BBB for CNS-related disease treatment. Mesenchymal stem cells (MSCs) are capable of migrating across endothelial cells by either the paracellular or transcellular pathway and subsequently preferentially home back to the site of inflammation or injury in the brain to exert their therapeutic effects. In a rat model of spinal cord injury, MRG-001 mobilized and recruited bone marrow stem cells into the injured spinal cord, these stem cells repaired/regenerated damaged spinal cord tissues and significantly improved the locomotor function. MRG-001 mobilized bone marrow stem cells exhibited a potential to regenerate neurons. If mobilized stem cells could migrate across the impaired BBB in ALS patients, and MRG-001 may promote repair/regeneration of damaged neurons through recruited bone marrow stem cells.

MRG-001 targets various aspects of ALS, including, but not limited to, down-regulating key pathogenesis pathways related to ALS, ameliorating systemic/neuroinflammation and promoting repair/regeneration of damaged motor neurons, thereby, providing an opportunity for treating and/or curing ALS.

Compassionate Use of MRG-001 in a Patient with ALS, Graves' Disease, and Drug-Induced Liver Injury

Patient Demographics: A 57-year-old male patient presented with progressive left arm and leg weakness beginning in August 2021. Subsequent diagnostic workup in September 2021 confirmed Amyotrophic Lateral Sclerosis (ALS). Despite treatment with standard ALS medications (riluzole, edaravone, AMX0035), there was no significant improvement, and the patient experienced a steady decline in function with an ALS Functional Rating Scale (ALSFRS) score of 20 in early November 2023. In October 2023, concurrent diagnoses of Graves' disease and drug-induced liver injury were made, with elevated liver enzymes (AST/ALT) and total bilirubin noted.

Treatment: The patient received 3 injections of MRG-001 (0.007-0.01 mL/kg/dose) per week for two weeks per month for a total of 5 months. Significant improvement was observed following the first round of MRG-001 treatment, including reduced muscle spasms and fasciculations, enabled assisted walking, strengthened limb muscles, reduced saliva production, improved tongue extension, and assisted turning over became possible.

Outcome: By June 2024, the ALSFRS score improved from 20 (November 2023) to 24, and body weight increased from 62.5 kg to 71 kg. The MRG-001 treatment not only halted the progression of ALS but also reversed several symptoms, significantly improving the patient's quality of life. These improvements included enhanced muscle strength, particularly in the legs, better sleep quality, reduced drooling, and increased grip and hand strength.

Graves' Disease Cure: The patient was diagnosed with Graves' disease, an immune system disorder causing the thyroid gland to produce too much thyroid hormone (hyperthyroidism). Prior to MRG-001 treatment, the patient's thyroid function tests showed abnormal results: T3: 3.57, FT3: 9.06, T4: 189, FT4: 37.33, TSH:0.01 (low, suggestive of hyperthyroidism). Following 3 months of treatment with MRG-001, thyroid function normalized: T3: 1.94, FT3: 4.96, T4: 149, FT4: 13.96, allowing the patient to stop taking anti-hyperthyroidism medication.

Improvement in Drug-Induced Liver Injury: The patient was diagnosed with drug-induced liver injury in October 2023, with increased liver enzymes and total bilirubin levels (total bilirubin 35, ALT 71, AST 40.9). Two months following MRG-001 treatment, liver enzymes and bilirubin levels returned to normal (total bilirubin 14.1, ALT 26, AST 19).

Conclusion: Compassionate use of MRG-001 in a 57-year-old male patient with ALS and concurrent Graves' disease and drug-induced liver injury demonstrated significant clinical improvement. MRG-001 therapy not only halted ALS progression and reversed symptoms, including improved motor function, but also normalized thyroid and liver function, resolving Graves' disease. The outcome of this patient with compassionate use of MRG-001 suggests that MRG-001 can treat neurodegenerative diseases (ALS), autoimmune diseases (Graves' disease), and liver injury.

1. A method to mobilize various stem and immune cells in a subject, comprising administering a pharmaceutical composition, wherein the pharmaceutical composition comprises
a) a stem cell mobilizer, and
b) an immunosuppressive agent or non-immunosuppressive FK binding protein ligand;
wherein the immunosuppressive agent or non-immunosuppressive FK binding protein ligand is administered at a sub-immunosuppressive dose.

2. The method of claim 1, wherein the stem cell mobilizer comprises AMD 3100.

3. The method of claim 2, wherein the dose range of AMD3100 is about 0.12 mg/kg to about 0.48 mg/kg.

4. The method of claim 2, wherein the AMD3100 plasma concentration range at about 1, about 3, and/or about 8 hours is about 200 ng/ml to about 1,000 ng/mL.

5. The method of claim 1, wherein the immunosuppressive agent or non-immunosuppressive FK binding protein ligand comprises Tacrolimus.

6. The method of claim 5, wherein the dose range of Tacrolimus is about 0.0025 mg/kg to about 0.01 mg/kg.

7. The method of claim 6, wherein the Tacrolimus plasma concentration range at about 1, about 3, about 8, about 12 and/or about 24 hours is about 1 ng/mL to about 7 ng/mL.

8. The method of claim 1, wherein stem cells that are mobilized comprise one or more of CD45^{Int}CD34+ hematopoietic stem cells (HSCs), CD45^{Int}CD34+CD90+ mesenchymal stem cells (MSCs), CD45^{Int}CD34+CD133+HSCs, CD45^{Int}CD34+CD133+CD31+ endothelial progenitor cells (EPCs), CD45^{Int}CD34+CD133+VEGFR2+ early EPCs, and CD45^{Int}SSEA3+ multi-lineage differentiating stress enduring cells (Muse cells).

9. The method of claim 1, wherein immune cells including immunoregulator cells that are mobilized comprise one or more of CD3+ T cells, CD3+CD4+ T cells, CD3+CD8+ T cells, CD3+/CD4+/Q3 CCR7+CD45RA− central memory CD4 T-lymphocytes, CD3+/CD8+/Q3 CCR7+CD45RA− central memory CD8 T-lymphocytes, CD3+/CD4+/Q4 CCR7−CD45RA− effector memory CD4 T-lymphocytes, CD3+/CD8+/Q4 CCR7−CD45RA− effector memory CD8 T-lymphocytes, CD19+B cells, CD3+CD4+Foxp3+ regulatory T cells (Tregs), and CD3+CD8+Foxp3+ Tregs.

10. A method to down-regulate molecular pathways for treating one or more of inflammation, allograft rejection and autoimmune disease in a subject, comprising administering a pharmaceutical composition to a subject, the pharmaceutical composition comprising:
a) a stem cell mobilizer, and
b) an immunosuppressive agent or non-immunosuppressive FK binding protein ligand;
wherein the immunosuppressive agent or non-immunosuppressive FK binding protein ligand is administered at a sub-immunosuppressive dose.

11. The method of claim 10, wherein the stem cell mobilizer comprises AMD 3100.

12. The method of claim 11, wherein the dose range of AMD3100 is about 0.12 mg/kg to about 0.48 mg/kg.

13. The method of claim 11, wherein the AMD3100 plasma concentration range at about 1, about 3, and/or about 8 hours is about 200 ng/ml to about 1,000 ng/mL.

14. The method of claim 10, wherein the immunosuppressive agent or non-immunosuppressive FK binding protein ligand comprises Tacrolimus.

15. The method of claim 14, wherein the dose range of Tacrolimus is about 0.0025 mg/kg to about 0.01 mg/kg.

16. The method of claim 15, wherein the Tacrolimus plasma concentration range at about 1, about 3, about 8, about 12 and/or about 24 hours is about 1 ng/mL to about 7 ng/mL.

17. The method of claim 10, wherein the inflammatory and autoimmune disease or disorder comprise one or more of Addison's Disease, Alopecia areata, ARDS, Ankylosing Spondylitis, Antiphospholipid Antibody Syndrome, Autoimmune Encephalitis, Autoimmune Hepatitis, Behcet's Disease, Bullous pemphigoid, Chronic Recurrent Multifocal Osteomyelitis, Diabetes Type I, Diverticulitis, Epidermolysis bullosa acquisita (EBA), Gout, Granulomatosis with polyangiitis, Graves Disease, Guillan-Barre Syndrome, Hashimoto's Thyroiditis, Henoch-Schonlein Purpura, IgA Nephropathy, Idiopathic pulmonary fibrosis, Juvenile Dermatomyositis, Juvenile Idiopathic Arthritis, Juvenile Lupus (SLE), Juvenile Scleroderma, Juvenile Vasculitis, Kawasaki Disease, Lambert-Eaton myasthenic syndrome (LEMS), Lichen planus, Lupus (Systemic Lupus Erythematosus), Meniere's disease, Mixed Connective Tissue Disease, Morvan's syndrome, Multiple Sclerosis, Myasthenia gravis, Myositis, Neuromyelitis Optica, Polymyalgia rheumatica, Poststreptococcal Inflammatory Syndromes, Primary biliary cholangitis, Primary sclerosing cholangitis, Psoriasis, Psoriatic Arthritis, Raynaud's phenomenon, Reactive Arthritis, Rheumatoid Arthritis, Sarcoidosis, Scleritis, Scleroderma, Sjogren's Syndrome, Spondyloarthritis, Spondyloarthropathy, Systemic Juvenile Idiopathic Arthritis, Stiff-person syndrome, Sweet's syndrome, Transverse myelitis, Undifferentiated Connective Tissue Disease, Uveitis, Vasculitis and Vitiligo.

18. The method of claim 10, wherein the allograft rejection comprises one or more of liver transplantation, corncal transplantation, intestinal transplantation, uterus transplantation, skin transplantation, kidney transplantation, lung transplantation, heart transplantation, pancreas and/or islet transplantation, bone marrow transplantation and vascularized composite allotransplantation (VCA).

19. The method of claim 10, wherein the down-regulated pathways comprise one or more of TNFalpha signaling via NFKB, inflammatory response, allograft rejection, IL-2 STAT5 signaling, IL-6 JAK/STAT3 signaling, oxidative phosphorylation, MTORC1 signaling, Hypoxia, P53, coagulation, apoptosis, complement, interferon gamma response and oxidative oxygen stress (ROS) pathway.

20. A method to down-regulate molecular pathways for one or more of: treating ischemia, reperfusion injury, neurodegenerative disease or disorder, inflammatory pain or disorder and steatohepatitis, anti-thrombosis, anti-aging, anti-fibrosis and anti-cancer, in a subject by administering a pharmaceutical composition comprising:
a) a stem cell mobilizer, and
b) an immunosuppressive agent or non-immunosuppressive FK binding protein ligand;
wherein the immunosuppressive agent or non-immunosuppressive FK binding protein ligand is administered at a sub-immunosuppressive dose.

21. The method of claim 20, wherein the stem cell mobilizer comprises AMD 3100.

22. The method of claim 21, wherein the dose range of AMD3100 is about 0.12 mg/kg to about 0.48 mg/kg.

23. The method of claim 21, wherein the AMD3100 plasma concentration range at about 1, about 3, and/or about 8 hours is about 200 ng/ml to about 1,000 ng/mL.

24. The method of claim 20, wherein the immunosuppressive agent or non-immunosuppressive FK binding protein ligand comprises Tacrolimus.

25. The method of claim 24, wherein the dose range of Tacrolimus is about 0.0025 mg/kg to about 0.01 mg/kg.

26. The method of claim 25, wherein the Tacrolimus plasma concentration range at about 1, about 3, about 8, about 12 and/or about 24 hours is about 1 ng/mL to about 7 ng/mL.

27. The method of claim 20, wherein the ischemia or reperfusion injury comprise one or more of Decubitus Ulcers (Pressure Sores), Gangrene, Ischemic heart disease (coronary heart disease or coronary artery disease), Hypoxic ischemic encephalopathy (HIE), Interstitial lung disease, Intestinal ischemia and ischemic colitis, Ischemic stroke, Limb ischemia (Peripheral Artery Disease), Myocardial Infarction, Renal Ischemia, Pulmonary infarction, Spinal cord ischemia and Transient Ischemic Attack.

28. The method of claim 27, wherein the down-regulated pathways comprise one or more of hypoxia, ROS pathway, P53 pathway, mTORC1 signaling pathway, apoptosis, TNFalpha signaling via NFKB, Inflammatory response, IL-2 STAT5 signaling, IL-6 JAK/STAT3 signaling and Oxidative phosphorylation.

29. The method of claim 20, wherein the anti-thrombotic effect comprises one or more of Deep vein thrombosis, Pulmonary embolism, Peripheral artery disease, Antiphopholipid syndrome, Thromboflebitis, Systemic Lupus Erythematosus and Inflammatory bowel diseases.

30. The method of claim 29, wherein the down-regulated pathways comprise one or more of Coagulation pathway, Complement pathway, Inflammatory response and ROS pathway.

31. The method of claim 20, wherein the anti-aging comprises one or more of Age-related macular degeneration, Alzheimer's Disease, Atherosclerosis, Cancers, Cataracts, Chronic Kidney Disease, Heart Failure, Huntington's Disease, Mild Cognitive Impairment, Type 2 Diabetes, Osteoarthritis, Osteoporosis, Parkinson's Disease, Presbycusis and Sarcopenia.

32. The method of claim 31, wherein the down-regulated pathways comprise one or more of mTORC1 signaling, IL-6 JAK/STAT3 signaling, Inflammatory response, P53 pathway, Apoptosis, ROS pathway and Unfolded protein response.

33. The method of claim 20, wherein the anti-fibrosis comprises any fibrosis caused by injury to organs and tissues, the injury comprising one or more of Liver Cirrhosis, Non-Alcoholic Steatohepatitis, Alcoholic Liver disease, Idiopathic Pulmonary fibrosis, Sarcoidosis, Myocardial fibrosis, Chronic Kidney Disease, Diabetic Nephropathy, Keloids, Hypertrophic scars, Chronic pancreatitis, Myelofibrosis, Retinal fibrosis and Retroperitoneal fibrosis.

34. The method of claim 33, wherein the down-regulated pathways comprise one or more of WNT beta-catenin signaling and TGF-beta signaling.

35. The method of claim 20, wherein the cancer comprises MYC expressing cancers.

36. The method of claim 35, wherein the cancer comprises one or more of breast cancer, lung cancer, colorectal cancers, prostate cancer, melanoma and non-melanoma skin cancers, bladder cancer, stomach cancer and liver cancer.

37. The method of claim 35, wherein the down-regulated pathways comprise one or more of MYC targets VI, MYC Targets V2, Kras signaling up, Androgen response, Peroxisome, DNA repair, unfolded protein response, ROS pathway, UV response DN, G2M Checkpoint and MTORC1 Signaling.

38. The method of claim 20, wherein the neurodegenerative disease or disorder comprises one or more of Chronic inflammatory demyelinating polyneuropathy (CIDP), dementia-type diseases, demyelinating diseases, frontotemporal dementia, Lewy body dementia, corticobasal degeneration, primary progressive aphasia, spinal muscular atrophy, Kennedy's disease, mitochondrial encephalomyopathy, lactic acidosis, and stroke-like episodes (MELAS), Leigh Syndrome, Krabbe Disease, Canavan Disease, Metachromatic Leukodystrophy (MLD), Adrenoleukodystrophy (ALS), Nieman-Pick Disease, Alexander Disease, Pelizaeus-Merzbacher Disease (PMD), Zellweger Syndrome, Parkinsonism-type diseases, Motor neuron diseases comprising one or more of amyotrophic lateral sclerosis (ALS) and progressive supranuclear palsy (PSP), multiple system atrophy, spinocerebellar ataxis and prion diseases.

39. The method of claim 38, wherein the down-regulated pathways comprise one or more of TNF-alpha Signaling, TGF-beta Signaling, Complement, WNT Beta Catenin, Hypoxia, IFN Gamma Response, Inflammatory response, IL-6 JAK Stat3 signaling, Unfold Protein Response, P53 pathway, Apoptosis pathway, Oxidative Phosphorylation, ROS pathway, KRAS signaling Up, Fatty Acid Metabolism, Heme metabolism, MTORC1 Signaling and Peroxisome.

40. The method of claim 20, wherein the inflammatory pain or disorder comprises one or more of pain secondary to any tissue injury caused by any etiology, rheumatoid arthritis, lower back pain, osteoarthritis pain, gout pain and neuropathic pain.

41. The method of claim 40, wherein the down-regulated pathways comprise one or more of IL-6 JAK Stat3 signaling, TNF-alpha Signaling, TGF-beta Signaling, IFN Gamma Response, Inflammatory response, oxidative phosphorylation and ROS pathway.

42. The method of claim 20, wherein the steatohepatitis comprises one or more of Non-alcoholic steatohepatitis (NASH) and Alcohol-associated steatohepatitis.

43. The method of claim 42, wherein the down-regulated pathways comprise one or more of TNF-alpha signaling via NFKB, ROS pathway, TGF-beta signaling, inflammatory response, IL-2 STAT5 signaling, IL-6 JAK/STAT3 signaling, oxidative phosphorylation, MTORC1 signaling, hypoxia, fatty acid metabolism, apoptosis, interferon gamma and xenobiotic metabolism.

44. A method to up-regulate interferon alpha response pathway for stimulating the antiviral and anti-cancer response in a subject comprising administering a pharmaceutical composition to a subject, wherein the pharmaceutical composition comprises
a) a stem cell mobilizer, and
b) an immunosuppressive agent or non-immunosuppressive FK binding protein ligand;
wherein the immunosuppressive agent or non-immunosuppressive FK binding protein ligand is administered at a sub-immunosuppressive.

45. The method of claim 44, wherein the stem cell mobilizer comprises AMD 3100.

46. The method of claim 45, wherein the dose range of AMD3100 is about 0.12 mg/kg to about 0.48 mg/kg.

47. The method of claim 45, wherein the AMD3100 plasma concentration range at about 1, about 3, and/or about 8 hours is about 200 ng/mL to about 1,000 ng/mL.

48. The method of claim 44, wherein the immunosuppressive agent or non-immunosuppressive FK binding protein ligand comprises Tacrolimus.

49. The method of claim 48, wherein the dose range of Tacrolimus is about 0.0025 mg/kg to about 0.01 mg/kg.

50. The method of claim 49, wherein the Tacrolimus plasma concentration range at about 1, about 3, about 8, about 12 and/or about 24 hours is about 1 ng/ml to about 7 ng/ml.

51. The method of claim 44, wherein the virus and/or cancer comprise one or more of various viruses' infection diseases comprising one or more of hepatitis B and C, and virus infection related cancers, lymphoma, malignant melanoma (skin cancer), genital warts, hairy cell leukemia (blood cell cancer), and Kaposi sarcoma (AIDS-related tumor).

52. The method of claim 44, wherein the up-regulated pathway comprises interferon alpha response.

53. A method to up-regulate heme metabolism pathway for promoting heme synthesis in a subject comprising administering a pharmaceutical composition to a subject, wherein the pharmaceutical composition comprises
a) a stem cell mobilizer, and
b) an immunosuppressive agent or sub-immunosuppressive FK binding protein ligand;
wherein the immunosuppressive agent or non-immunosuppressive FK binding protein ligand is administered at a sub-immunosuppressive dose.

54. The method of claim 53, wherein the stem cell mobilizer comprises AMD 3100.

55. The method of claim 54, wherein the dose range of AMD3100 is about 0.12 mg/kg to about 0.48 mg/kg.

56. The method of claim 54, wherein the AMD3100 plasma concentration range at about 1, about 3, and/or about 8 hours is about 200 ng/ml to about 1,000 ng/mL.

57. The method of claim 53, wherein the immunosuppressive agent or non-immunosuppressive FK binding protein ligand comprises Tacrolimus.

58. The method of claim 57, wherein the dose range of Tacrolimus is about 0.0025 mg/kg to about 0.01 mg/kg.

59. The method of claim 58, wherein the Tacrolimus plasma concentration range at about 1, about 3, about 8, about 12 and/or about 24 hours is about 1 ng/mL to about 7 ng/mL.

60. The method of claim 53, wherein the promotion of heme synthesis comprises a deficiency in an enzyme or substrate that leads to an accumulation or increase of intermediates of heme synthesis in blood, tissues, and/or urine for treatment of a group of disorders comprising porphyria.

61. The method of claim 53, wherein the up-regulated pathway comprises heme metabolism.
